(19) United States
(12) Patent Application Publication
(10) Pub. No.: US 2011/0257198 A1
(43) Pub. Date: Oct. 20, 2011

(54) PIPERIDINE DERIVATIVES USEFUL AS OREXIN ANTAGONISTS

(76) Inventors: Giuseppe Alvaro, Verona (IT); David Amantini, Verona (IT)

(21) Appl. No.: 13/141,388

(22) PCT Filed: Dec. 21, 2009
(86) PCT No.: PCT/EP2009/067658

§ 371 Date: Jun. 22, 2011

(30) Foreign Application Priority Data
Dec. 23, 2008 (GB) 0823467.6

(51) Int. Cl. A61K 31/506; C07D 471/04; A61K 31/437; A61P 9/00; A61P 25/22; A61P 25/30; A61P 25/04; A61P 11/00; C07D 401/14; A61P 25/00

(52) U.S. Cl. 514/256; 544/333; 546/121; 514/300

(57) ABSTRACT

Disclosed are imidazopyridylmethylene substituted piperidine derivatives of Formula (I):

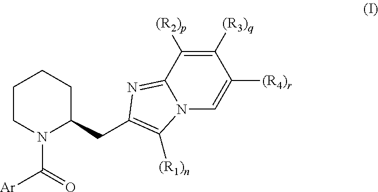

useful as orexin antagonists.

This invention relates to imidazopyridylmethylene substituted piperidine derivatives and their use as pharmaceuticals.

Many medically significant biological processes are mediated by proteins participating in signal transduction pathways that involve G-proteins and/or second messengers.

Polypeptides and polynucleotides encoding the human 7-transmembrane G-protein coupled neuropeptide receptor, orexin-1 (HFGAN72), have been identified and are disclosed in EP875565, EP875566 and WO 96/34877. Polypeptides and polynucleotides encoding a second human orexin receptor, orexin-2 (HFGANP), have been identified and are disclosed in EP893498.

Polypeptides and polynucleotides encoding polypeptides which are ligands for the orexin-1 receptor, e.g. orexin-A (Lig72A) are disclosed in EP849361.

The orexin ligand and receptor system has been well characterised since its discovery (see for example Sakurai, T. et al (1998) Cell, 92 pp 573 to 585; Smart et al (1999) British Journal of Pharmacology 128 pp 1 to 3; Willie et al (2001) Ann. Rev. Neurosciences 24 pp 429 to 458; Sakurai (2007) Nature Reviews Neuroscience 8 pp 171 to 181; Ohno and Sakurai (2008) Front. Neuroendocrinology 29 pp 70 to 87). From these studies it has become clear that orexins and orexin receptors play a number of important physiological roles in mammals and open up the possibility of the development of new therapeutic treatments for a variety of diseases and disorders as described hereinbelow.

Experiments have shown that central administration of the ligand orexin-A stimulated food intake in freely-feeding rats during a 4 hour time period. This increase was approximately four-fold over control rats receiving vehicle. These data suggest that orexin-A may be an endogenous regulator of appetite (Sakurai, T. et al (1998) Cell, 92 pp 573 to 585; Peyron et al (1998) J. Neurosciences 18 pp 9996 to 10015; Willie et al (2001) Ann. Rev. Neurosciences 24 pp 429 to 458). Therefore, antagonists of the orexin-A receptor(s) may be useful in the treatment of obesity and diabetes. In support of this it has been shown that orexin receptor antagonist SB334867 potently reduced hedonic eating in rats (White et al (2005) Peptides 26 pp 2231 to 2238) and also attenuated high-fat pellet self-administration in rats (Nair et al (2008) British Journal of Pharmacology, published online 28 Jan. 2008). The search for new therapies to treat obesity and other eating disorders is an important challenge. According to WHO definitions a mean of 35% of subjects in 39 studies were overweight and a further 22% clinically obese in westernised societies. It has been estimated that 5.7% of all healthcare costs in the USA are a consequence of obesity. About 85% of Type 2 diabetics are obese. Diet and exercise are of value in all diabetics. The incidence of diagnosed diabetes in westernised countries is typically 5% and there are estimated to be an equal number undiagnosed. The incidence of obesity and Type 2 diabetes is rising, demonstrating the inadequacy of current treatments which may be either ineffective or have toxicity risks including cardiovascular effects. Treatment of diabetes with sulfonylureas or insulin can cause hypoglycaemia, whilst metformin causes GI side-effects. No drug treatment for Type 2 diabetes has been shown to reduce the long-term complications of the disease. Insulin sensitisers will be useful for many diabetics, however they do not have an anti-obesity effect.

As well as having a role in food intake, the orexin system is also involved in sleep and wakefulness. Rat sleep/EEG studies have shown that central administration of orexin-A, an agonist of the orexin receptors, causes a dose-related increase in arousal, largely at the expense of a reduction in paradoxical sleep and slow wave sleep 2, when administered at the onset of the normal sleep period (Hagan et al (1999) Proc. Natl. Acad. Sci. 96 pp 10911 to 10916). The role of the orexin system in sleep and wakefulness is now well established (Sakurai (2007) Nature Reviews Neuroscience 8 pp 171 to 181; Ohno and Sakurai (2008) Front. Neuroendocrinology 29 pp 70 to 87; Chemelli et al (1999) Cell 98 pp 437 to 451; Lee et al (2005) J. Neuroscience 25 pp 6716 to 6720; Piper et al (2000) European J Neuroscience 12 pp 726-730 and Smart and Jerman (2002) Pharmacology and Therapeutics 94 pp 51 to 61). Antagonists of the orexin receptors may therefore be useful in the treatment of sleep disorders including insomnia. Studies with orexin receptor antagonists, for example SB334867, in rats (see for example Smith et al (2003) Neuroscience Letters 341 pp 256 to 258) and more recently dogs and humans (Brisbare-Roch et al (2007) Nature Medicine 13(2) pp 150 to 155) further support this.

In addition, recent studies have suggested a role for orexin antagonists in the treatment of motivational disorders, such as disorders related to reward seeking behaviours for example drug addiction and substance abuse (Borgland et al (2006) Neuron 49(4) pp 589-601; Boutrel et al (2005) Proc. Natl. Acad. Sci. 102(52) pp 19168 to 19173; Harris et al (2005) Nature 437 pp 556 to 559).

International Patent Applications WO99/09024, WO99/58533, WO00/47577 and WO00/47580 disclose phenyl urea derivatives and WO00/47576 discloses quinolinyl cinnamide derivatives as orexin receptor antagonists. WO05/118548 discloses substituted 1,2,3,4-tetrahydroisoquinoline derivatives as orexin antagonists.

WO01/96302, WO02/44172, WO02/89800, WO03/002559, WO03/002561, WO03/032991, WO03/037847, WO03/041711 and WO08/038,251, WO09/003,993, WO09/003,997 and WO09/124,956 all disclose cyclic amine derivatives.

WO03/002561 discloses N-aroyl cyclic amine derivatives as orexin antagonists. Compounds disclosed in WO03/002561 include piperidine derivatives substituted at the 2-position with bicyclic heteroarylmethyl groups. We have now found that some piperidine derivatives substituted at the 2-position with an imidazo[1,2-c]pyridin-2-ylmethyl group have beneficial properties including, for example, increased oral bioavailability and significantly increased solubility in physiologically relevant media compared to the prior art compounds. Such properties make these imidazo[1,2-c]pyridin-2-ylmethyl substituted piperidine derivatives very attractive as potential pharmaceutical agents which may be useful in the prevention or treatment of obesity, including obesity observed in Type 2 (non-insulin-dependent) diabetes patients, sleep disorders, anxiety, depression, schizophrenia, drug dependency or compulsive behaviour. Additionally these compounds may be useful in the treatment of stroke, particularly ischemic or haemorrhagic stroke, and/or blocking the emetic response, i.e. useful in the treatment of nausea and vomiting.

Accordingly the present invention provides a compound of formula (I)

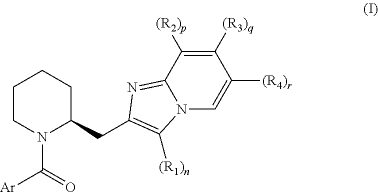

where:
Ar is pyridinyl substituted with one, two or three groups independently selected from the group consisting of C₁₋₄alkyl, halo, C₁₋₄alkoxy, haloC₁₋₄alkyl, haloC₁₋₄alkoxy, cyano, phenyl or a 5 or 6 membered heterocyclyl group containing 1, 2 or 3 atoms selected from N, O or S, which phenyl or heterocyclyl group is optionally substituted with C₁₋₄alkyl, halo, C₁₋₄alkoxy, haloC₁₋₄alkyl, haloC₁₋₄alkoxy or cyano;
R₁ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR⁵R⁶ wherein R⁵ is H or (C₁₋₄)alkyl and R⁶ is H or (C₁₋₄)alkyl;
R₂ is (C₁₋₄)alkyl, (C₁₋₄)alkenyl, HO(C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR⁷R⁸ wherein R⁷ is H or (C₁₋₄)-alkyl and R⁸ is H or (C₁₋₄)-alkyl;
R₃ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR⁹R¹⁰ wherein R⁹ is H or (C₁₋₄-alkyl and R¹⁰ is H or (C₁₋₄)-alkyl;
R₄ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR¹¹R¹² wherein R¹¹ is H or (C₁₋₄)-alkyl and R¹² is H or (C₁₋₄)-alkyl;
n is 0 or 1;
p is 0 or 1;
q is 0 or 1;
r is 0 or 1;
or a pharmaceutically acceptable salt thereof.

In one embodiment Ar is pyridinyl substituted with one, two or three groups independently selected from the group consisting of C₁₋₄alkyl, halo, C₁₋₄alkoxy, haloC₁₋₄alkyl, haloC₁₋₄alkoxy, cyano or phenyl;

R₁ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR⁵R⁶ wherein R⁵ is H or (C₁₋₄)alkyl and R⁶ is H or (C₁₋₄)alkyl;
R₂ is (C₁₋₄)alkyl, (C₁₋₄)alkenyl, HO(C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR⁷R⁸ wherein R⁷ is H or (C₁₋₄)-alkyl and R⁸ is H or (C₁₋₄)-alkyl;
R₃ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR⁹R¹⁰ wherein R⁹ is H or (C₁₋₄)-alkyl and R¹⁰ is H or (C₁₋₄)-alkyl;
R₄ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR¹¹R¹² wherein R¹¹ is H or (C₁₋₄)-alkyl and R¹² is H or (C₁₋₄)-alkyl;
n is 0 or 1;
p is 0 or 1;
q is 0 or 1;
r is 0 or 1;
or a pharmaceutically acceptable salt thereof.

In one embodiment the pyridyl group is linked to the carbonyl group by means of a bond formed between the carbon at the 2 position of the pyridyl and the carbon of said carbonyl group.

In one embodiment the pyridyl group is linked to the carbonyl group by means of a bond formed between the carbon at the 3 position of the pyridyl and the carbon of said carbonyl group.

In one embodiment the pyridyl group is linked to the carbonyl group by means of a bond formed between the carbon at the 4 position of the pyridyl and the carbon of said carbonyl group.

In one embodiment the pyridyl group is linked to the carbonyl group by means of a bond formed between the nitrogen at the 1 position of the pyridyl and the carbon of said carbonyl group.

In one embodiment Ar is substituted with one (C₁₋₄)alkyl group and one (C₁₋₄)alkoxy group.

In another embodiment Ar is substituted with one methyl group and one (C₁₋₄)alkoxy group.

In one embodiment Ar is substituted with one (C₁₋₄)alkyl group and one propoxy, ethoxy, methoxy, methylethoxy, methylpropoxy or cyclopropylmethoxy group.

In one embodiment Ar is substituted with one methyl group and one propoxy, ethoxy, methoxy, methylethoxy, methylpropoxy or cyclopropylmethoxy group.

In one embodiment Ar is substituted with one (C₁₋₄)alkyl group and one phenyl group.

In one embodiment Ar is substituted with one methyl group and one phenyl group.

In one embodiment q is 1 and R₃ is alkyl.

In another embodiment q is 1 and R₃ is methyl.

In one embodiment p is 1 and R₂ is alkyl.

In another embodiment p is 1 and R₂ is methyl.

In one embodiment n is 0, p is 1, q is 1, r is 0, R₂ is alkyl, R₃ is alkyl and Ar is substituted with one (C₁₋₄)alkyl group and one (C₁₋₄)alkoxy group.

In another embodiment n is 0, p is 1, q is 1, r is 0, R₂ is methyl, R₃ is methyl and Ar is substituted with one methyl group and one propoxy group.

In one embodiment the pyridyl group is linked to the carbonyl group by means of a bond formed between the carbon at the 2 position of the pyridyl and the carbon of said carbonyl group, n is 0, p is 1, q is 1, r is 0, R₂ is alkyl, R₃ is alkyl and Ar is substituted with one (C₁₋₄)alkyl group and one (C₁₋₄)alkoxy group.

In another embodiment the pyridyl group is linked to the carbonyl group by means of a bond formed between the carbon at the 2 position of the pyridyl and the carbon of said carbonyl group, n is 0, p is 1, q is 1, r is 0, R₂ is methyl, R₃ is methyl and Ar is substituted with one methyl group and one propoxy group.

In one embodiment the pyridyl group is linked to the carbonyl group by means of a bond formed between the carbon at the 2 position of the pyridyl and the carbon of said carbonyl group, n is 0, p is 1, q is 0, r is 1, R₂ is (C₁₋₄)alkyl, R₄ is halo and Ar is substituted with one (C₁₋₄)alkyl group and one phenyl group.

In another embodiment the pyridyl group is linked to the carbonyl group by means of a bond formed between the carbon at the 2 position of the pyridyl and the carbon of said carbonyl group, n is 0, p is 1, q is 0, r is 1, R₂ is methyl, R₄ is fluoro and Ar is substituted with one methyl group and one phenyl group.

In one embodiment the pyridyl group is linked to the carbonyl group by means of a bond formed between the carbon at the 2 position of the pyridyl and the carbon of said carbonyl group, n is 1, p is 1, q is 0, r is 0, R₁ is halo, R₂ is (C₁₋₄)alkyl and Ar is substituted with one (C₁₋₄)alkyl group and one cyclopropoxymethyl group.

In one embodiment the pyridyl group is linked to the carbonyl group by means of a bond formed between the carbon at the 2 position of the pyridyl and the carbon of said carbonyl group, n is 1, p is 1, q is 0, r is 0, R₁ is chloro, R₂ is methyl and Ar is substituted with one methyl group and one cyclopropoxymethyl group.

In one embodiment the invention provides the compound of formula (I) selected from the group consisting of:
2-[((2S)-1-{[3-(ethyloxy)-6-methyl-2-pyridinyl]carbonyl}-2-piperidinyl)methyl]-6-fluoro-8-methylimidazo[1,2-a]pyridine;
6-fluoro-8-methyl-2-{[(2S)-1-({6-methyl-3-[(2-methylpropyl)oxy]-2-pyridinyl}carbonyl)-2-piperidinyl]methyl}imidazo[1,2-c]pyridine;
6,8-dimethyl-2-{[(2S)-1-({6-methyl-3-[(2-methylpropyl)oxy]-2-pyridinyl}carbonyl)-2-piperidinyl]methyl}imidazo[1,2-c]pyridine;
8-methyl-2-[((2S)-1-{[6-methyl-3-(propyloxy)-2-pyridinyl]carbonyl}-2-piperidinyl)methyl]imidazo[1,2-a]pyridine;
2-{[(2S)-1-({3-[(cyclopropylmethyl)oxy]-6-methyl-2-pyridinyl}carbonyl)-2-piperidinyl]methyl}-8-methylimidazo[1,2-a]pyridine;
8-methyl-2-{[(2S)-1-({6-methyl-3-[(1-methylethyl)oxy]-2-pyridinyl}carbonyl)-2-piperidinyl]methyl}imidazo[1,2-a]pyridine;
2-[((2S)-1-{[4-chloro-3-(ethyloxy)-6-methyl-2-pyridinyl]carbonyl}-2-piperidinyl)methyl]-8-methylimidazo[1,2-a]pyridine;
7,8-dimethyl-2-{[(2S)-1-({6-methyl-3-[(2-methylpropyl)oxy]-2-pyridinyl}carbonyl)-2-piperidinyl]methyl}imidazo[1,2-c]pyridine;
2-{[(2S)-1-({3-[(cyclopropylmethyl)oxy]-6-methyl-2-pyridinyl}carbonyl)-2-piperidinyl]methyl}-7,8-dimethylimidazo[1,2-c]pyridine;
2-[((2S)-1-{[3-(ethyloxy)-6-methyl-2-pyridinyl]carbonyl}-2-piperidinyl)methyl]-7,8-dimethylimidazo[1,2-c]pyridine;
7,8-dimethyl-2-[((2S)-1-{[6-methyl-3-(propyloxy)-2-pyridinyl]carbonyl}-2-piperidinyl)methyl]imidazo[1,2-a]pyridine;
8-fluoro-2-[((2S)-1-{[6-methyl-3-(propyloxy)-2-pyridinyl]carbonyl}-2-piperidinyl)methyl]imidazo[1,2-c]pyridine;
8-fluoro-2-{[(2S)-1-({6-methyl-3-[(2-methylpropyl)oxy]-2-pyridinyl}carbonyl)-2-piperidinyl]methyl}imidazo[1,2-c]pyridine;
2-{[(2S)-1-({3-[(cyclopropylmethyl)oxy]-6-methyl-2-pyridinyl}carbonyl)-2-piperidinyl]methyl}-8-fluoroimidazo[1,2-c]pyridine;
6,7-dimethyl-2-{[(2S)-1-({6-methyl-3-[(2-methylpropyl)oxy]-2-pyridinyl}carbonyl)-2-piperidinyl]methyl}imidazo[1,2-a]pyridine;
3-chloro-2-{[(2S)-1-({3-[(cyclopropylmethyl)oxy]-6-methyl-2-pyridinyl}carbonyl)-2-piperidinyl]methyl}-8-methylimidazo[1,2-a]pyridine;
3-chloro-2-[((2S)-1-{[3-(ethyloxy)-6-methyl-2-pyridinyl]carbonyl}-2-piperidinyl)methyl]-8-methylimidazo[1,2-a]pyridine;
2-{[(2S)-1-({3-[(cyclopropylmethyl)oxy]-6-methyl-2-pyridinyl}carbonyl)-2-piperidinyl]methyl}-3,8-dimethylimidazo[1,2-a]pyridine;
2-[((2S)-1-{[6-ethyl-3-(ethyloxy)-2-pyridinyl]carbonyl}-2-piperidinyl)methyl]-7,8-dimethylimidazo[1,2-a]pyridine;
6-fluoro-8-methyl-2-({(2S)-1-[(6-methyl-3-phenyl-2-pyridinyl)carbonyl]-2-piperidinyl}methyl)imidazo[1,2-c]pyridine;
7,8-dimethyl-2-[((2S)-1-{[6-methyl-3-(3-methyl-1,2,4-oxadiazol-5-yl)-2-pyridinyl]carbonyl}-2-piperidinyl)methyl]imidazo[1,2-a]pyridine;
7,8-dimethyl-2-[((2S)-1-{[6-methyl-3-(5-methyl-1,3-oxazol-2-yl)-2-pyridinyl]carbonyl}-2-piperidinyl)methyl]imidazo[1,2-a]pyridine;
2-[((2S)-1-{[3-(5-ethyl-1,3-oxazol-2-yl)-6-methyl-2-pyridinyl]carbonyl}-2-piperidinyl)methyl]-7,8-dimethylimidazo[1,2-a]pyridine;
7,8-dimethyl-2-[((2S)-1-{[6-methyl-3-(2-pyrimidinyl)-2-pyridinyl]carbonyl}-2-piperidinyl)methyl]imidazo[1,2-a]pyridine;
7,8-dimethyl-2-[((2S)-1-{[6-methyl-3-(3-methyl-5-isoxazolyl)-2-pyridinyl]carbonyl}-2-piperidinyl)methyl]imidazo[1,2-a]pyridine;
7,8-dimethyl-2-[((2S)-1-{[6-methyl-3-(4-methyl-1,3-thiazol-2-yl)-2-pyridinyl]carbonyl}-2-piperidinyl)methyl]imidazo[1,2-a]pyridine; and
6-fluoro-8-methyl-2-[((2S)-1-{[6-methyl-3-(2-pyrimidinyl)-2-pyridinyl]carbonyl}-2-piperidinyl)methyl]imidazo[1,2-c]pyridine
   or a pharmaceutically acceptable salt thereof.

When the compound contains a C₁₋₄alkyl group, whether alone or forming part of a larger group, e.g. C₁₋₄alkoxy, the alkyl group may be straight chain, branched or cyclic, or combinations thereof. Examples of C₁₋₄alkyl are methyl or ethyl. An example of C₁₋₄alkoxy is methoxy.

Examples of haloC₁₋₄alkyl include trifluoromethyl (i.e. —CF₃).

Examples of C₁₋₄alkoxy include methoxy and ethoxy.

Examples of haloC₁₋₄alkoxy include trifluoromethoxy (i.e. —OCF₃).

Halogen or “halo” (when used, for example, in haloC₁₋₄)alkyl means fluoro, chloro, bromo or iodo.

It is to be understood that the present invention covers all combinations of particularised groups and substituents described herein above.

It will be appreciated that for use in medicine the salts of the compounds of formula (I) should be pharmaceutically acceptable. Suitable pharmaceutically acceptable salts will be apparent to those skilled in the art. Pharmaceutically acceptable salts include those described by Berge, Bighley and Monkhouse J. Pharm. Sci (1977) 66, pp 1-19. Such pharmaceutically acceptable salts include acid addition salts formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid. Other salts e.g. oxalates or formates, may be used, for example in the isolation of compounds of formula (I) and are included within the scope of this invention.

Certain of the compounds of formula (I) may form acid addition salts with one or more equivalents of the acid. The present invention includes within its scope all possible stoichiometric and non-stoichiometric forms.

The compounds of formula (I) may be prepared in crystalline or non-crystalline form and, if crystalline, may optionally be solvated, eg. as the hydrate. This invention includes within its scope stoichiometric solvates (eg. hydrates) as well as compounds containing variable amounts of solvent (eg. water).

It will be understood that the invention includes pharmaceutically acceptable derivatives of compounds of formula (I) and that these are included within the scope of the invention.

As used herein “pharmaceutically acceptable derivative” includes any pharmaceutically acceptable ester or salt of such ester of a compound of formula (I) which, upon administration to the recipient is capable of providing (directly or indirectly) a compound of formula (I) or an active metabolite or residue thereof.

The compounds of formula (I) are S enantiomers. Where additional chiral centres are present in compounds of formula (I), the present invention includes within its scope all possible enantiomers and diastereoisomers, including mixtures thereof. The different isomeric forms may be separated or resolved one from the other by conventional methods, or any given isomer may be obtained by conventional synthetic methods or by stereospecific or asymmetric syntheses. The invention also extends to any tautomeric forms or mixtures thereof.

The subject invention also includes isotopically-labeled compounds which are identical to those recited in formula (I) but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, fluorine, iodine and chlorine such as ³H, ¹¹C, ¹⁴C, ¹⁸F, ¹²³I or ¹²⁵I.

Compounds of the present invention and pharmaceutically acceptable salts of said compounds that contain the aforementioned isotopes and/or other isotopes of other atoms are within the scope of the present invention. Isotopically labeled compounds of the present invention, for example those into which radioactive isotopes such as ³H or ¹⁴C have been incorporated, are useful in drug and/or substrate tissue distribution assays. Tritiated, ie. ³H, and carbon-14, ie. ¹⁴C, isotopes are particularly preferred for their ease of preparation and detectability. ¹¹C and ¹⁸F isotopes are particularly useful in PET (positron emission tomography).

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

According to a further aspect of the present invention there is provided a process for the preparation of compounds of formula (I) and derivatives thereof. The following schemes detail some synthetic routes to compounds of the invention. In the following schemes reactive groups can be protected with protecting groups and deprotected according to well established techniques.

### Schemes

According to a further feature of the invention there is provided a process for the preparation of compounds of formula (I) or salts thereof. The following is an example of a synthetic scheme that may be used to synthesise the compounds of the invention.

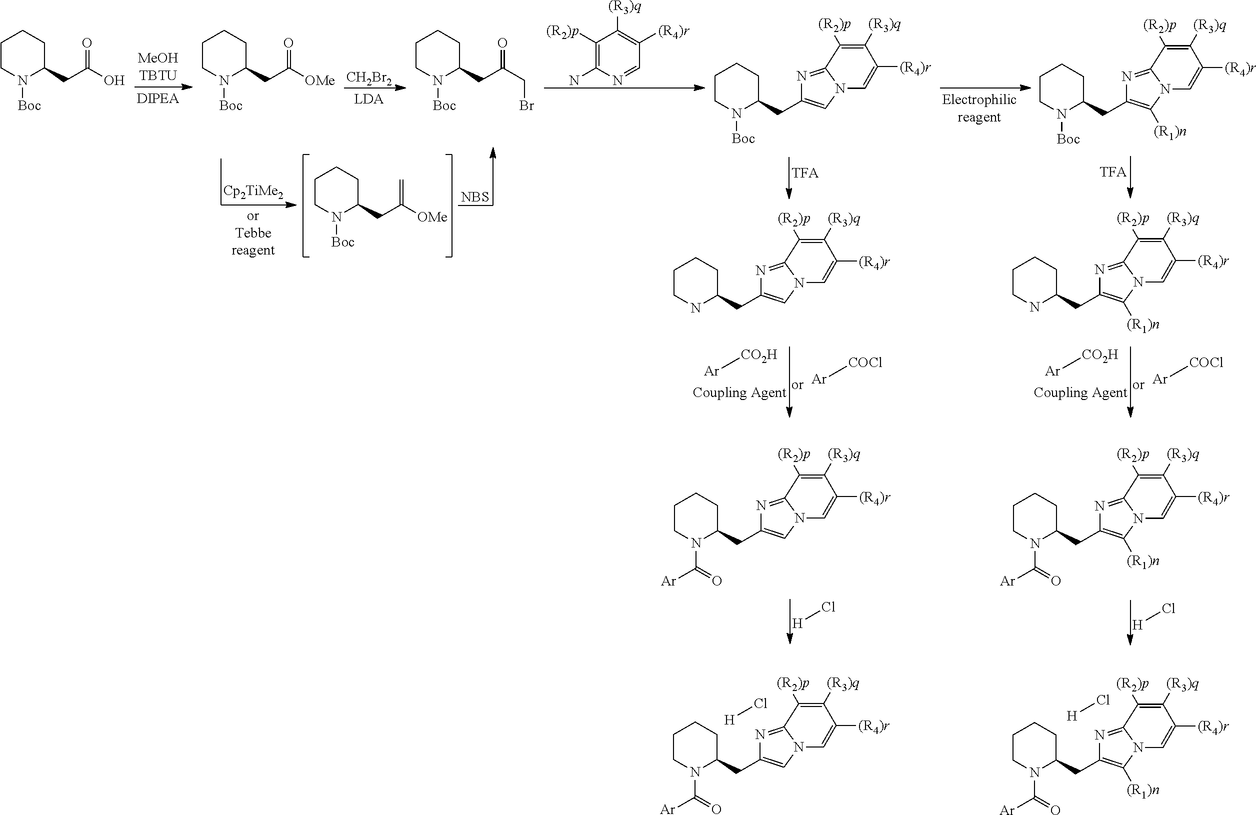

It will be understood by those skilled in the art that certain compounds of the invention can be converted into other compounds of the invention according to standard chemical methods.

The starting materials for use in the scheme are commercially available, known in the literature or can be prepared by known methods. ((2S)-1-{[(1,1-dimethylethyl)oxy]carbonyl}-2-piperidinyl)acetic acid is available from Neosystem Product List (BA19302).

Pharmaceutically acceptable salts may be prepared conventionally by reaction with the appropriate acid or acid derivative.

The present invention provides compounds of formula (I) or a pharmaceutically acceptable salt thereof for use in human or veterinary medicine.

The compounds of formula (I) or their pharmaceutically acceptable salts may be of use for the treatment or prophylaxis of a disease or disorder where an antagonist of a human orexin receptor is required such as sleep disorders selected from the group consisting of Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; Sleep Apnea and Jet-Lag Syndrome.

In one embodiment compounds of formula (I) or their pharmaceutically acceptable salts may be of use for the treatment or prophylaxis of Primary Insomnia (307.42), Circadian Rhythm Sleep Disorder (307.45) and Dyssomnia Not Otherwise Specified (307.47), Sleep Disorders Related to Another Mental Disorder such as Insomnia Related to Another Mental Disorder (307.42) and Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type.

In addition the compounds of formula (I) or their pharmaceutically acceptable salts may be of use for the treatment or prophylaxis of a disease or disorder where an antagonist of a human orexin receptor is required such as depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

Further, the compounds of formula (I) or their pharmaceutically acceptable salts may be of use for the treatment or prophylaxis of a disease or disorder where an antagonist of a human orexin receptor is required such as anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00).

In addition the compounds of formula (I) or their pharmaceutically acceptable salts may be of use for the treatment or prophylaxis of a disease or disorder where an antagonist of a human orexin receptor is required such as substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

In addition the compounds of formula (I) or their pharmaceutically acceptable salts may be of use for the treatment or prophylaxis of a disease or disorder where an antagonist of a human orexin receptor is required such as feeding disorders such as bulimia nervosa, binge eating, obesity, including obesity observed in Type 2 (non-insulin-dependent) diabetes patients. Further, the compounds of formula (I) or their pharmaceutically acceptable salts may be of use for the treatment or prophylaxis of a disease or disorder where an antagonist of a human orexin receptor is required such as stroke, particularly ischemic or haemorrhagic and/or in blocking an emetic response i.e. nausea and vomiting.

The numbers in brackets after the listed diseases refer to the classification code in DSM-IV: Diagnostic and Statistical Manual of Mental Disorders, 4th Edition, published by the American Psychiatric Association. The various subtypes of the disorders mentioned herein are contemplated as part of the present invention.

The invention also provides a method for the treatment of a disease or disorder where an antagonist of a human orexin receptor is required, for example those diseases and disorders mentioned hereinabove, in a subject in need thereof, comprising administering to said subject an effective amount of a compound of formula (I), or a pharmaceutically acceptable salt thereof.

The invention also provides a compound of formula (I), or a pharmaceutically acceptable salt thereof, for use in the treatment or prophylaxis of a disease or disorder where an antagonist of a human orexin receptor is required, for example those diseases and disorders mentioned hereinabove.

The invention also provides the use of a compound of formula (I), or a pharmaceutically acceptable salt thereof, in the manufacture of a medicament for use in the treatment or prophylaxis of a disease or disorder where an antagonist of a human Orexin receptor is required, for example those diseases and disorders mentioned hereinabove.

For use in therapy the compounds of the invention are usually administered as a pharmaceutical composition. The invention also provides a pharmaceutical composition comprising a compound of formula (I), or a pharmaceutically acceptable salt thereof, and a pharmaceutically acceptable carrier.

The compounds of formula (I) or their pharmaceutically acceptable salts may be administered by any convenient method, e.g. by oral, parenteral, buccal, sublingual, nasal, rectal or transdermal administration, and the pharmaceutical compositions adapted accordingly.

The compounds of formula (I) or their pharmaceutically acceptable salts which are active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a fluorochlorohydrocarbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

In one embodiment the composition is in unit dose form such as a tablet, capsule or ampoule.

The composition may contain from 0.1% to 100% by weight, for example from 10 to 60% by weight, of the active material, depending on the method of administration. The composition may contain from 0% to 99% by weight, for example 40% to 90% by weight, of the carrier, depending on the method of administration. The composition may contain from 0.05 mg to 1000 mg, for example from 1.0 mg to 500 mg, of the active material, depending on the method of administration. The composition may contain from 50 mg to 1000 mg, for example from 100 mg to 400 mg of the carrier, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 500 mg, and such unit doses may be administered more than once a day, for example two or three a day. Such therapy may extend for a number of weeks or months.

Orexin-A (Sakurai, T. et al (1998) Cell, 92 pp 573-585) can be employed in screening procedures for compounds which inhibit the ligand's activation of the orexin-1 or orexin-2 receptors.

In general, such screening procedures involve providing appropriate cells which express the orexin-1 or orexin-2 receptor on their surface. Such cells include cells from mammals, yeast, Drosophila or E. coli. In particular, a polynucleotide encoding the orexin-1 or orexin-2 receptor is used to transfect cells to express the receptor. The expressed receptor is then contacted with a test compound and an orexin-1 or orexin-2 receptor ligand, as appropriate, to observe inhibition of a functional response. One such screening procedure involves the use of melanophores which are transfected to express the orexin-1 or orexin-2 receptor, as described in WO 92/01810.

Another screening procedure involves introducing RNA encoding the orexin-1 or orexin-2 receptor into Xenopus oocytes to transiently express the receptor. The receptor oocytes are then contacted with a receptor ligand and a test compound, followed by detection of inhibition of a signal in the case of screening for compounds which are thought to inhibit activation of the receptor by the ligand.

Another method involves screening for compounds which inhibit activation of the receptor by determining inhibition of binding of a labelled orexin-1 or orexin-2 receptor ligand to cells which have the orexin-1 or orexin-2 receptor (as appropriate) on their surface. This method involves transfecting a eukaryotic cell with DNA encoding the orexin-1 or orexin-2 receptor such that the cell expresses the receptor on its surface and contacting the cell or cell membrane preparation with a compound in the presence of a labelled form of an orexin-1 or orexin-2 receptor ligand. The ligand may contain a radioactive label. The amount of labelled ligand bound to the receptors is measured, e.g. by measuring radioactivity.

Yet another screening technique involves the use of FLIPR equipment for high throughput screening of test compounds that inhibit mobilisation of intracellular calcium ions, or other ions, by affecting the interaction of an orexin-1 or orexin-2 receptor ligand with the orexin-1 or orexin-2 receptor as appropriate.

Throughout the specification and claims which follow, unless the context requires otherwise, the word ‘comprise’, and variations such as ‘comprises’ and ‘comprising’ will be understood to imply the inclusion of a stated integer or step or group of integers but not to the exclusion of any other integer or step or group of integers or steps.

All publications, including but not limited to patents and patent applications, cited in this specification are herein incorporated by reference as if each individual publication were specifically and individually indicated to be incorporated by reference herein as though fully set forth.

The following Examples illustrate the preparation of certain compounds of formula (I) or salts thereof. The Descriptions 1 to 87 illustrate the preparation of intermediates used to make compounds of formula (I) or salts thereof.

In the procedures that follow, after each starting material, reference to a description is typically provided. This is provided merely for assistance to the skilled chemist. The starting material may not necessarily have been prepared from the Description referred to.

The yields were calculated assuming that products were 100% pure if not stated otherwise.

The compounds described in the Examples described hereinafter have all been prepared as a first step from stereo chemically pure 1,1-dimethylethyl (2S)-2-[2-(methyloxy)-2-oxoethyl]-1-piperidinecarboxylate. The stereochemistry of the compounds of the Descriptions and Examples have been assigned on the assumption that the pure configuration is maintained.

Compounds are named using ACD/Name PRO 6.02 chemical naming software (Advanced Chemistry Development Inc., Toronto, Ontario, M5H2L3, Canada).

Proton Magnetic Resonance (NMR) spectra were recorded either on Varian instruments at 400, 500 or 600 MHz, or on a Bruker instrument at 400 MHz. Chemical shifts are reported in ppm (δ) using the residual solvent line as internal standard. Splitting patterns are designed as s, singlet; d, doublet; t, triplet; q, quartet; m, multiplet; b, broad. The NMR spectra were recorded at a temperature ranging from 25 to 90° C. When more than one conformer was detected the chemical shifts for the most abundant one is usually reported.

Unless otherwise specified, HPLC analyses indicated by HPLC (walk-up): rt (retention time)=x min, were performed on a Agilent 1100 series instrument using a Luna 3u C18(2) 100A column (50×2.0 mm, 3 μm particle size) [Mobile phase and Gradient: 100% (water+0.05% TFA) to 95% (acetonitrile+0.05% TFA) in 8 min. Column T=40° C. Flow rate=1 mL/min. UV detection wavelength=220 nm]. Other HPLC analyses, indicated by HPLC (walk-up, 3 min method), were performed using an Agilent Zorbax SB-C18 column (50×3.0 mm, 1.8 μm particle size) [Mobile phase and Gradient: 100% (water+0.05% TFA) to 95% (acetonitrile+0.05% TFA) in 2.5 min, hold 0.5 min. Column T=60° C. Flow rate=1.5 mL/min. UV detection wavelength=220 nm].

In the analytical characterization of the described compounds “MS” refers to Mass Spectra taken by Direct infusion Mass or to Mass Spectra associated with peaks taken by HPLC/MS or HPLC/MS analysis, where the Mass Spectrometer used is as mentioned below.

Direct infusion Mass spectra (MS) were run on a Agilent MSD 1100 Mass Spectrometer, operating in ES (+) and ES (−) ionization mode [ES (+): Mass range: 100-1000 amu. Infusion solvent: water+0.1% HCO₂H/CH₃CN 50/50. ES (−): Mass range: 100-1000 amu. Infusion solvent: water+0.05% NH₄OH/CH₃CN 50/50] MS spectra associated with the peaks were taken on HPLC instrument Perkin Elmer 200 series coupled to an Applied Biosystems API150EX Mass Spectrometer. UV and MS spectra associated with the peaks were taken on HPLC instrument Agilent 1100 Series coupled to an Agilent LC/MSD 1100 Mass Spectrometer operating in positive or negative electrospray ionization mode and in both acidic and basic gradient conditions [Acidic gradient LC/MS—ES (+ or −): analyses performed on a Supelcosil ABZ+Plus column (33×4.6 mm, 3 μm). Mobile phase: A—water+0.1% HCO₂H/B—CH₃CN. Gradient (standard method): t=0 min 0% (B), from 0% (B) to 95% (B) in 5 min lasting for 1.5 min, from 95% (B) to 0% (B) in 0.1 min, stop time 8.5 min. Column T=room temperature. Flow rate=1 mL/min. Gradient (fast method): t=0 min 0% (B), from 0% (B) to 95% (B) in 3 min lasting for 1 min, from 95% (B) to 0% (B) in 0.1 min, stop time 4.5 min. Column T=room temperature. Flow rate=2 mL/min.

Basic gradient LC/MS—ES (+ or −): analyses performed on a XTerra MS C18 column (30×4.6 mm, 2.5 μm). Mobile phase: A—5 mM aq. NH₄HCO₃+ammonia (pH 10)/B—CH₃CN. Gradient: t=0 min 0% (B), from 0% (B) to 50% (B) in 0.4 min, from 50% (B) to 95% (B) in 3.6 min lasting for 1 min, from 95% (B) to 0% (B) in 0.1 min, stop time 5.8 min. column temperature=room temperature. Flow rate=1.5 mL/min]. Mass range ES (+ or −): 100-1000 amu. UV detection range: 220-350 nm. The usage of this methodology is indicated by “LC-MS” in the analytic characterization of the described compounds.

Total ion current (TIC) and DAD UV chromatographic traces together with MS and UV spectra associated with the peaks were taken on a HPLC/MS Acquity™ system equipped with 2996 PDA detector and coupled to a Waters Micromass ZQ™ Mass Spectrometer operating in positive or negative electrospray ionisation mode [LC/MS—ES (+ or −): analyses performed using an Acquity™ HPLC BEH C18 column (50×21 mm, 1.7 μm particle size), column temperature 40° C.]. Mobile phase: A—water+0.1% HCOOH/B—CH₃CN+0.075% HCOOH, Flow rate: 1.0 mL/min, Gradient: t=0 min 3% B, t=0.05 min 6% B, t=0.57 min 70% B, t=1.4 min 99% B, t=1.45 min 3% B). The usage of this methodology is indicated by “HPLC” in the analytic characterization of the described compounds.

[LC/MS—ES (+ or −): analyses performed using an Acquity™ UPLC BEH C18 column (50×2.1 mm, 1.7 μm particle size) column temperature 40° C.]. Mobile phase: A—water+0.1% HCO₂H/B—CH₃CN+0.06% or 0.1% HCO₂H. Gradient: t=0 min 3% B, t=1.5 min 100% B, t=1.9 min 100% B, t=2 min 3% B stop time 2 min. Column T=40° C. Flow rate=1.0 mL/min. Mass range: ES (+): 100-1000 amu or ES(+): 50-800 amu. ES (−): 100-800 amu. UV detection range: 210-350 nm. The usage of this methodology is indicated by “HPLC (Acid IPQC)” in the analytic characterization of the described compounds. [LC/MS—ES (+ or −): analyses performed using an Acquity™ UPLC BEH C18 column (50×2.1 mm, 1.7 μm particle size) column temperature 40° C.]. Mobile phase: A—water+0.1% HCO₂H/B—CH₃CN+0.06% or 0.1% HCO₂H. Gradient: t=0 min 3% B, t=0.05 min 6% B, t=0.57 min 70% B, t=1.06 min 99% B lasting for 0.389 min, t=1.45 min 3% B, stop time 1.5 min. Column T=40° C. Flow rate=1.0 mL/min. Mass range: ES (+): 100-1000 amu or ES(+): 50-800 amu, ES (−): 100-800 amu. UV detection range: 210-350 nm. The usage of this methodology is indicated by “HPLC (Acid QC_POS_{—}50-800 or QC_POS_{—}70_{—}900 or GEN_QC or FINAL_QC)” in the analytic characterization of the described compounds.

[LC/MS—ES (+ or −): analyses performed using an Acquity™ UPLC BEH C18 column (50×2.1 mm, 1.7 μm particle size) column temperature 40° C.]. Mobile phase: A—water+0.1% HCO₂H/B—CH₃CN+0.06% or 0.1% HCO₂H. Gradient: t=0 min 3% B, t=1.06 min 99% B, t=1.45 min 99% B, t=1.46 min 3% B, stop time 1.5 min. Column T=40° C. Flow rate=1.0 mL/min. Mass range: ES (+): 100-1000 amu. ES (−): 100-800 amu. UV detection range: 210-350 nm. The usage of this methodology is indicated by “HPLC (Acid GEN_QC_SS)” in the analytic characterization of the described compounds.

Total ion current (TIC) and DAD UV chromatographic traces together with MS and UV spectra associated with the peaks were taken on a HPLC/MS Acquity™ system equipped with PDA detector and coupled to a Waters SQD mass spectrometer operating in positive and negative alternate electrospray ionisation mode [LC/MS—ES(+ or −): analyses performed using an Acquity™ UPLC BEH C18 column (50×2.1 mm, 1.7 μm particle size) column temperature 40° C.]. Mobile phase: A—10 mM aqueous solution of NH₄HCO₃ (adjusted to pH 10 with ammonia)/B—CH₃CN. Gradient: t=0 min 3% B, t=1.06 min 99% B lasting for 0.39 min, t=1.46 min 3% B, stop time 1.5 min. Column T=40° C. Flow rate=1.0 mL/min. Mass range: ES (+): 100-1000 amu or ES (+): 50-800 amu. ES (−): 100-1000 amu. UV detection range: 220-350 nm. The usage of this methodology is indicated by “UPLC (Basic GEN_QC or QC_POS_{—}50-800)” in the analytic characterization of the described compounds.

Unless otherwise specified, Preparative LC-MS purifications were run on a MDAP (Mass Detector Auto Purification) Waters instrument (MDAP FractionLynx). [LC/MS—ES (+): analyses performed using a Gemini C18 AXIA column (50×21 mm, 5 μm particle size). Mobile phase: A—NH₄HCO₃ sol. 10 mM, pH 10; B—CH₃CN. Flow rate: 17 ml/min]. The gradient will be specified each time:

Preparative LC-MS purifications were also run on a MDAP (Mass Detector Auto Purification) Waters instrument. The usage of this methodology is indicated by “Fraction Lynx” in the analytic characterization of the described compounds. Sunfire Prep. C18 OBD (150 mm×30 min i.d. 5 μm particle size) at room temperature. The injection volume was: 990 μl. Mobile phase: A=0.1% v/v solution of HCO₂H in water. B=0.1% v/v solution of HCO₂H in CH₃CN. Flow rate: 40 ml/min.

For reactions involving microwave irradiation, a Personal Chemistry Emrys™ Optimizer was used.

In a number of preparations, purification was performed using Biotage manual flash chromatography (Flash+), Biotage automatic flash chromatography (Horizon, SP1 and SP4), Companion CombiFlash (ISCO) automatic flash chromatography, Flash Master Personal or Vac Master systems.

Flash chromatography was carried out on silica gel 230-400 mesh (supplied by Merck AG Darmstadt, Germany), Varian Mega Be—Si pre-packed cartridges, pre-packed Biotage silica cartridges (e.g. Biotage SNAP cartridge), KP-NH prepacked flash cartridges or ISCO RediSep Silica cartridges.

SPE-SCX cartridges are ion exchange solid phase extraction columns supplied by Varian. The eluent used with SPE-SCX cartridges is DCM and MeOH or ACN or MeOH followed by 2 N ammonia solution in MeOH. The collected fractions are those eluted with the ammonia solution in MeOH.

SPE-Si cartridges are silica solid phase extraction columns supplied by Varian.

The following table lists the used abbreviations:

| | |
|---|---|
| AcCl | Acetyl chloride |
| AcOH | Acetic acid |
| bs or br.s | broad signal |
| BINAP | 2,2′-Bis(diphenylphosphino)-1,1′-binaphthyl |
| Boc | t-Butoxycarbonyl |
| n-BuLi | n-Butyl lithium |
| Burgess reagent | Methyl N-(triethylammoniumsulphonyl)carbamate |
| Cp | Cyclopentadienyl |
| Cy | Cyclohexanes |
| DBA | Dibenzyilidene acetone |
| DCE | Dichloroethane |
| DCM | Dichloromethane |
| DIPEA | N,N-Diisopropyl-N-ethylamine |
| DMSO | Dimethylsulfoxide |
| DIPA | N,N-Diisopropylamine |
| DMAE | 2-(Dimethylamino)ethanol |
| DME | 1,2-Dimethoxyethane |
| DMF | Dimethylformamide |
| EtOH | Ethanol |
| Et₂O | Diethylether |
| EtOAc | Ethylacetate |
| IPA | Isopropyl alcohol |
| LAH | Lithium aluminum hydride |
| LDA | Lithiumdiisopropylamide |
| MeOH | Methanol |
| EtOH | Ethanol |
| Grubbs 1^{st} | Benzylidene- |
| generation | bis(tricyclohexylphosphine)dichlororuthenium |
| MsCl | Mesylchloride |
| NBS | N-Bromosuccinimide |
| NCS | N-Chlorosuccinimide |
| Ps-TsCl | Polystyrene sulfonyl chloride (cross-linked polystyrene |
| | resin that is the resin-bound equivalent of tosyl |
| | chloride) |
| rt | retention time |
| TBME | tert-Butyl methyl ether |
| TBS | tert-Butyl dimethylsilyl |
| TBTU | O-(benzotriazol-1-yl)-N,N,N′N′-tetramethyluronium |
| | tetrafluoroborate |
| TEA | Triethylamine |
| TFA | Trifluoroacetic acid |
| THF | Tetrahydrofuran |
| bs | broad signal |
| Ph | Phenyl |
| pH = 3 buffer | Citric acid/NaOH/HCl in water solution available from |
| solution | Merck KGaA |
| TBDPS | tert-Butyl diphenylsilyl |
| TBTU | O-(benzotriazol-1-yl)-N,N,N′N′-tetramethyluronium |
| | tetrafluoroborate |
| t-Bu | tert-Butyl |
| TEA | Triethylamine |
| TFA | Trifluoroacetic acid |
| THF | Tetrahydrofuran |
| Ts | p-Toluensulfonyl |

### DESCRIPTIONS

### Description 1

### 1,1-Dimethylethyl (2S)-2-[2-(methyloxy)-2-oxoethyl]-1-piperidinecarboxylate (D1)

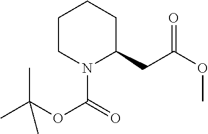

A mixture of ((2S)-1-{[(1,1-dimethylethyl)oxy]carbonyl}-2-piperidinyl)acetic acid (1.00 g, 4.11 mmol), DIPEA (2.148 ml, 12.33 mmol) and TBTU (1.979 g, 6.17 mmol) in DMF (25 ml) was stirred at room temperature for 20 minutes and a brown colour was formed. After this time MeOH (0.249 ml, 6.17 mmol) was added and the resulting solution stirred at room temperature for 30 minutes. The mixture was transferred into a separatory funnel containing brine (20 ml) and extracted with EtOAc (2×20 ml). The combined organic layers were washed with water/ice (5×20 ml). The organic layer was dried (Na₂SO₄), filtered and concentrated. The crude obtained was purified by flash chromatography on silica gel (Biotage SP1, Cy/EtOAc from 100/0 to 85/15). Collected fractions gave the title compound D1 (1.01 g) as a colorless oil.

¹H-NMR (400 MHz, CDCl₃) δ ppm: 4.67-4.75 (m, 1H), 3.96-4.05 (m, 1H), 3.67 (s, 3 H), 2.79 (t, 1H), 2.61 (dd, 1H), 2.53 (dd, 1H), 1.60-1.70 (m, 6H), 1.46 (s, 9H).

### Description 2

### 1,1-Dimethylethyl (2S)-2-(3-bromo-2-oxopropyl)-1-piperidinecarboxylate (D2)

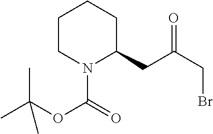

### Preparation (i)

In a 500 ml round-bottom flask under nitrogen at room temperature, 1,1-dimethylethyl (2S)-2-[2-(methyloxy)-2-oxoethyl]-1-piperidinecarboxylate D1 (11.10 g) was dissolved in THF (100 ml) to give a pale yellow solution. This solution was cooled to −78° C. and the Tebbe reagent (104 ml of a 0.5 M solution in toluene, 51.80 mmol) was added dropwise. The thick mixture was diluted with further 70 ml of dry toluene. The resulting brown-orange mixture was stirred at −78° C. for 30 minutes and then slowly warmed up to room temperature and left under stirring for 2 hours. The reaction mixture was charged into a dropping funnel and then added dropwise to a 2 L round-bottom flask containing about 400 ml of an ice-cooled 1 M NaOH aqueous solution. At the end of the quench, the resulting grey suspension was diluted with EtOAc (250 ml) and allowed to stir overnight. The resulting yellow suspension was then filtered over a Gooch funnel and salts were washed with EtOAc (500 ml). Phases were then separated and the organic layer was washed with brine (2×500 ml). The organic phase was dried (Na₂SO₄), filtered and concentrated to give a deep orange oil. The residue was diluted with Et₂O (about 500 ml). Some salts precipitated and the resulting suspension was filtered over a Gooch funnel. The filtrate was concentrated under vacuum to give 12.40 g of 1,1-dimethylethyl (2S)-2-[2-(methyloxy)-2-propen-1-yl]-1-piperidinecarboxylate as an orange-brown crude oil. The material contained some residual salts (the overall recovered amount was higher than the theoretical amount). The material was used without further purification in the next reaction and supposed to be pure at 88.7 wt %. In a 1 L round-bottom flask under nitrogen at room temperature 1,1-dimethylethyl (2S)-2-[2-(methyloxy)-2-propen-1-yl]-1-piperidinecarboxylate (12.40 g, 43.10 mmol) was dissolved in THF (125 ml) and water (35 ml) to give a pale yellow solution. NBS (7.67 g, 43.10 mmol) was then added dissolved in about 100 ml of THF. The resulting grey mixture was stirred at room temperature for 1 hour. Additional NBS (1.50 g, 0.2 eq) dissolved in 50 ml of THF was added and the reaction mixture stirred at room temperature for 1 hours. The mixture was concentrated under vacuum to remove THF, and then was diluted with EtOAc (about 500 ml) and water (200 ml). Phases were separated and the aqueous layer was back-extracted with EtOAc (250 ml). The combined organic layers were dried (Na₂SO₄), filtered and concentrated to give 17.80 g of a brown oil. The material was purified by flash chromatography on silica gel (Biotage 75 L, Cy/EtOAc from 100/0 to 90/10) to give the title compound D2 (6.00 g) as a yellow oil. MS: (ES/+) m/z: 342 (M+Na, 100%) and 344 (M+Na, 100%), 264 (M-tBu, 100%) and 266 (M-tBu, 100%). C₁₃H₂₂BrNO₃ requires 319. ¹H NMR (400 MHz, CDCl₃) δ ppm: 4.72-4.79 (m, 1H), 3.91-4.10 (m, 3 H), 2.77-2.97 (m, 3H), 1.49-1.75 (m, 6H), 1.46 (s, 9H).

### Alternative Preparation (ii)

An alternative route to (1,1-dimethylethyl (2S)-2-(3-bromo-2-oxopropyl)-1-piperidinecarboxylate) D2 is the following:

A stirred solution of DIPA (7.84 ml, 56.00 mmol) in THF (70 ml) was cooled to 0° C. and n-BuLi (35.70 ml of a 1.6 M solution in Cy, 57.10 mmol) was added dropwise. To a solution of dibromomethane (3.58 ml, 51.30 mmol) in THF (70 ml) cooled to −90° C. was added dropwise the LDA solution previously prepared. After 5 minutes stirring, a solution of 1,1-dimethylethyl (2S)-2-[2-(methyloxy)-2-oxoethyl]-1-piperidinecarboxylate D1 (6.00 g) in THF (47 ml) was added dropwise to the reaction mixture and then, after 10 minutes, n-BuLi (22.20 ml of a 1.6 M solution in Cy, 35.50 mmol) was added. After 5 minutes the resulting mixture was added, via cannula, to a rapidly stirring solution of AcCl (35.00 ml, 492 mmol) in absolute EtOH (230 ml) cooled to −78° C. The reaction mixture was left under stirring and then diluted with Et₂O (400 ml). The mixture was transferred into a separatory funnel and washed with a cold 10% H₂SO₄ aqueous solution (2×100 ml), a 5% NaHCO₃ aqueous solution (100 ml) and brine (100 ml). The organic phase was dried (Na₂SO₄), filtered and the solvent removed under reduced pressure. Purification by flash chromatography on silica gel (Biotage SP1 40 M, DCM) gave the title compound D2 (1.14 g).

Alternative Preparation (iii)

In a 1 L round-bottom flask titanocene dichloride (60 g, 0.24 mol) was suspended in dry toluene (300 ml) under nitrogen atmosphere and cooled down to 0° C. Methylmagnesium chloride (3 M solution in THF, 180 ml, 0.54 mol) was added dropwise (over 45 min), keeping the internal temperature below 8° C. The resulting mixture was stirred at 0-5° C. for 1.5 hours and then transferred (over 30 min) through a siphon in an ice-cooled 6% w/w NH₄Cl aqueous solution (180 ml), keeping the internal temperature below 5° C. The mixture was stirred at 0-5° C. for 1 hour. Celite (15 g) was added, the mixture stirred at 10° C. for 15 minutes and then filtered washing with toluene (20 ml). Phases were separated. The organic layer was washed with water (180 ml) and brine (180 ml), dried (Na₂SO₄), filtered and then distilled down under vacuo to 200 ml. The dimethyltitanocene solution in toluene was charged in a 1 L round-bottom flask under nitrogen atmosphere and 1,1-dimethylethyl (2S)-2-[2-(methyloxy)-2-oxoethyl]-1-piperidinecarboxylate D1 (20 g, 0.078 mol) was added. The resulting mixture was stirred at 90° C. for 3 hours. Toluene (500 ml) and iso-octane (500 ml) were added and the mixture filtered through a celite pad to remove inorganic salts. A CUNO filtration (R55S cartridge) was then performed to remove the finest particle size solid. The resulting clear solution was concentrated under vacuo to afford the intermediate 1,1-dimethylethyl (2S)-2-{2-[(methyloxy)methyl]-2-propen-1-yl}-1-piperidinecarboxylate as an orange oil (13.60 g). HPLC (walk-up): rt=4.69 min. ¹H-NMR (400 MHz, CDCl₃) δ ppm: 4.42-4.58 (m, 1H), 3.94-4.08 (m, 1H), 3.88-3.93 (m, 2H), 3.53 (s, 3H), 2.79 (t, 1H), 2.42 (dd, 1H), 2.27 (dd, 1H), 1.50-1.70 (m, 6H), 1.46 (s, 9H).

NBS (8.36 g, 0.047 mol) was added portionwise to a mixture of 1,1-dimethylethyl (2S)-2-{2-[(methyloxy)methyl]-2-propen-1-yl}-1-piperidinecarboxylate (10 g) in THF (70 ml) and H₂O (15 ml). The mixture was diluted with TBME (100 ml) and water (50 ml). The aqueous phase was back-extracted with TBME (50 ml). The collected organic phases were washed (twice) with a 4% w/w NaHCO₃ aqueous solution, dried (Na₂SO₄), filtered and evaporated under vacuo. The residual oil was purified by filtration through a silica pad (20 g, toluene/EtOAc 90/10). A further filtration through a silica pad (50 g, toluene/TBME 90/10) afforded the title compound D2 (7.80 g). ¹H-NMR (600 MHz, DMSO-d₆) δ ppm: 4.50-4.64 (m, 1H), 4.35 (s, 2H), 3.70-3.88 (m, 1H), 2.86-3.01 (m, 1H), 2.65-2.82 (m, 2H), 1.42-1.60 (m, 5H), 1.35 (s, 9H), 1.14-1.28 (m, 1H).

### Description 3

### 1,1-Dimethylethyl (2S)-2-[(8-methylimidazo[1,2-a]pyridin-2-yl)methyl]-1-piperidinecarboxylate (D3)

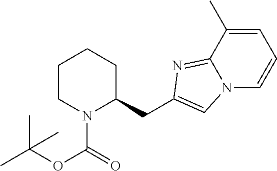

In a 50 ml round-bottom flask at room temperature under nitrogen, 1,1-dimethylethyl (2S)-2-(3-bromo-2-oxopropyl)-1-piperidinecarboxylate D2 (0.12 g) was dissolved in DMF (2 ml) to give a pale yellow solution. 3-Methyl-2-pyridinamine (0.0608 g, 0.562 mmol) was then added and the resulting solution heated at 80° C. for 45 minutes. The mixture was allowed to cool down to room temperature and was diluted with brine (5 ml) and Et₂O (2 ml). Phases were separated and the aqueous layer extracted with Et₂O (3×3 ml). The combined organic layers were dried (Na₂SO₄), filtered and concentrated to give 0.12 g of a crude pale yellow oil containing the title compound D3. The material was used without further purification in the next step. MS: (ES/+) m/z: 330 (M+1). C₁₉H₂₇N₃O₂ requires 329.

### Description 4

### 8-Methyl-2-[(2S)-2-piperidinylmethyl]imidazo[1,2-a]pyridine (D4)

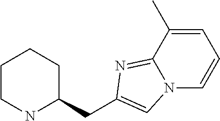

In a 100 ml pear flask 1,1-dimethylethyl (2S)-2-[(8-methylimidazo[1,2-a]pyridin-2-yl)methyl]-1-piperidinecarboxylate D3 (1.70 g) was dissolved in DCM (30 ml) to give a yellow solution that was cooled to 0° C. TFA (5 ml) was added dropwise and the resulting mixture left under stirring overnight. The mixture was evaporated under vacuum and the crude dark oil was eluted through a SCX column. Collected fractions gave the title compound D4 (1.05 g) as an oil. HPLC (walk-up): rt=1.85 min. MS: (ES/+) m/z: 230 (M+1). C₁₄H₁₉N₃ requires 229. ¹H NMR (400 MHz, CDCl₃) δ ppm: 7.94 (d, 1H), 7.41 (s, 1 H), 6.94 (d, 1H), 6.66 (t, 1H), 2.89-3.06 (m, 1H), 2.93-3.01 (m, 2H), 2.71-2.79 (m, 1H), 2.58-2.67 (m, 4H), 1.85-1.95 (bs, NH), 1.75-1.84 (m, 2H), 1.58-1.64 (m, 1H), 1.22-1.55 (m, 3H).

### Description 5

### 1,1-Dimethylethyl (2S)-2-[(3-iodo-8-methylimidazo[1,2-a]pyridin-2-yl)methyl]-1-piperidinecarboxylate (D5)

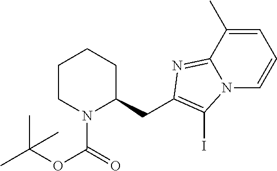

To a solution of 1,1-dimethylethyl (2S)-2-[(8-methylimidazo[1,2-a]pyridin-2-yl)methyl]-1-piperidinecarboxylate D3 (0.135 g) in DCM (50 ml) was added I₂ (12.91 ml, 12.91 mmol) (solution 1 M in DCM) dropwise at room temperature and the resulting mixture was stirred at room temperature for 3 hours. 5% NaHSO₃ aqueous solution (20 ml) was added and the mixture was vigorously stirred for 10 minutes. The organic phase was separated, dried, filtered and concentrated to give a yellow solid purified via Biotage SP4 (NH 12+M column; eluted with a gradient of 35 CV of Cy/EtOAc from 1/0 to 8/2) to afford the title compound D5 (0.132 g). HPLC (walk up): rt=3.82 min. MS: (ES/+) m/z: 456 (M+1). C₁₉H₂₆IN₃O₂ requires 455. ¹H NMR (400 MHz, DMSO-d₆) δ ppm: 8.2-8.0 (m, 1H), 7.2-7.0 (m, 1H), 7.0-6.8 (m, 1H), 4.6-4.3 (m, 1H), 4.0-3.8 (m, 1H), 3.2-2.7 (m, 3H), 2.48-2.44 (m, 3H), 1.8-0.54 (m, 15H).

### Description 6

### 1,1-Dimethylethyl (2S)-2-[(3,8-dimethylimidazo[1,2-a]pyridin-2-yl)methyl]-1-piperidinecarboxylate (D6)

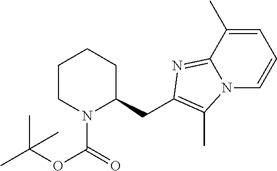

To a mixture of 1,1-dimethylethyl (2S)-2-[(3-iodo-8-methylimidazo[1,2-a]pyridin-2-yl)methyl]-1-piperidinecarboxylate D5 (0.100 g) and Tetrakis(triphenylphosphine)Palladium(0) (12.69 mg, 10.98 μmol) in DME (2 ml) was added methyl boronic acid (0.0197 g, 0.329 mmol) followed by the addition of NaOH 0.5 M solution (0.878 ml, 0.439 mmol). The resulting mixture was submitted to a microwave cycle at 110° C. for 40 min. The reaction was poured into water (2 ml) and extracted with DCM (3×2 ml). The organic phase was separated, filtered, dried and the solvent was evaporated. The brown residue was purified by flash chromatography (Biotage, NH 12+M; eluted with a gradient of Cy/EtOAc from 1/0 to 8/2). The title compound D6 (0.070 g) was obtained like colourless oil. MS: (ES/+) m/z: 344 (M+1). C₂₀H₂₉N₃O₂ requires 343.

### Description 7

### 3,8-Dimethyl-2-[(2S)-2-piperidinylmethyl]imidazo[1,2-a]pyridine (D7)

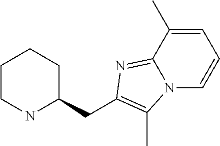

To a solution of 1,1-dimethylethyl (2S)-2-[(3,8-dimethylimidazo[1,2-a]pyridin-2-yl)methyl]-1-piperidinecarboxylate D6 (0.070 g) in DCM (2 ml) was added TFA (0.5 ml, 6.49 mmol) and the resulting mixture was stirred for 4 hours at room temperature. The volatiles were evaporated under reduced pressure and the residue was purified via SCX (5 g; eluted with 3 CV of MeOH and then with 4 CV of 2 M NH₃/MeOH). The basic fractions were joined together and the solvent was removed under reduced pressure to obtain a mixture of desired compound and compound without methyl group in position 3 (MW=229). This mixture was sent for preparative HPLC chromatography. The acid solution from the HPLC purification was made basic with Na₂CO₃ and extracted with DCM (3×5 ml), separated through a phase separator cartridge and evaporated under reduced pressure to obtain the title compound D7 (0.027 g) like white solid. HPLC (walk-up): rt=1.95 min. MS: (ES/+) m/z: 244 (M+1). C₁₅H₂₁N₃ requires 243. ¹H NMR (400 MHz, DMSO-d₆) δ ppm: 8.03-7.99 (m, 1H), 6.98-6.94 (m, 1H), 6.80-6.75 (m, 1H), 2.92-2.86 (m, 1H), 2.74-2.60 (m, 3H), 2.47-2.35 (m, 7H), 1.72-1.0 (m, 6H).

### Description 8

### 1,1-Dimethylethyl (2S)-2-[(8-fluoroimidazo[1,2-a]pyridin-2-yl)methyl]-1-piperidinecarboxylate (D8)

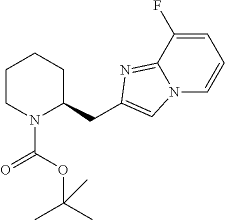

1,1-Dimethylethyl (2S)-2-(3-bromo-2-oxopropyl)-1-piperidinecarboxylate D2 (42.80 g) and 3-fluoro-2-pyridinamine (14.98 g, 134 mmol) were dissolved in dry DMF (240 ml) and the resulting solution was stirred at 80° C. for 4 hours. The reaction mixture was cooled to 25° C. and was diluted with saturated NaHCO₃ aqueous solution/water 1/1 (470 ml) and extracted with Et₂O (3×941 ml). The organic layers were combined, dried (Na₂SO₄) and the solvent removed under reduced pressure. The residue was purified by flash chromatography on silica gel (Biotage 75 L column, Cy/EtOAc/MeOH from 80/20/2.5 to 80/20/10) to afford 25.70 g of the title compound D8 contaminated with 3-fluoro-2-pyridinamine (25% from NMR analysis). The material was dissolved in DCM (650 ml). Ps-TsCl [38 g, 74.90 mmol (resin capacity 1.97 mmol/g)] and then DMAP (3 g, 24.56 mmol) were added. The resulting mixture was stirred at room temperature under Argon atmosphere overnight and filtered. The filtrate was dried (Na₂SO₄), the solvent removed under vacuum and the crude purified by flash chromatography on silica gel (Biotage 75 L column, Cy/EtOAc/MeOH from 80/20/2 to 80/20/5) to afford the title compound D8 (23.56 g). C₁₈H₂₄FN₃O₂ requires 333. ¹H NMR (400 MHz, CDCl₃) δ ppm: 7.86 (d, 1H), 7.40-7.57 (bs, 1H), 6.79-6.90 (m, 1 H), 6.60-6.71 (m, 1H), 4.63-4.77 (m, 1H), 3.97-4.16 (m, 1H), 3.18-3.34 (m, 1H), 2.86-3.03 (m, 2H), 1.33-1.81 (m, 6H), 1.13-1.37 (bs, 9H).

### Description 9

### 8-Fluoro-2-[(2S)-2-piperidinylmethyl]imidazo[1,2-a]pyridine hydrochloride (D9)

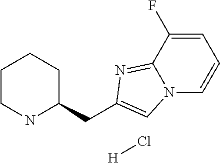

1,1-Dimethylethyl (2S)-2-[(8-fluoroimidazo[1,2-a]pyridin-2-yl)methyl]-1-piperidinecarboxylate D8 (23.56 g) was dissolved in DCM (35 ml) and the resulting solution cooled to 10° C. under Argon atmosphere. A 4 M HCl solution in 1,4-dioxane (148 ml, 594 mmol) was added dropwise, the reaction allowed to warm-up to room temperature and left under stirring for 2.15 hours. Volatiles were removed under vacuo and the residue triturated with Et₂O (2×250 ml) to give the title compound D9 (23.796 g) as a white solid. The material contained some residual 1,4-dioxane and 3-fluoro-2-pyridinamine (the overall recovered amount was higher than the theoretical amount) and was used in the next step without further purification. MS: (ES/+) m/z: 234 (M+1-HCl). C₁₃H₁₇FClN₃ requires 269.

### Description 10

### 2-Chloro-5-fluoro-3-methylpyridine (D10)

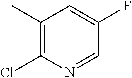

To a −20° C. cooled solution of (2-chloro-5-fluoro-3-pyridinyl)methanol (3.086 g, 19.10 mmol) and TEA (5.32 ml, 38.20 mmol) in anhydrous DCM (180 ml), MsCl (2.233 ml, 28.70 mmol) was added dropwise and the resulting reaction mixture stirred at 0° C. for 30 minutes. Volatiles were evaporated under reduced pressure to afford the desired mesylate (4.53 g) that was used in the next step without further purification. [Mesylate data: MS: (ES/+) m/z: 240 (M+1) and 242 (M+1). C₇H₇ClFNO₃S requires 239].

To an ice-cooled mixture of the crude mesylate (4.53 g, 18.90 mmol) in THF (180 ml), LAH (18.90 ml of a 1.0 M solution in THF, 18.90 mmol) was added dropwise and the reaction was stirred for 1 hour. A 2 M HCl aqueous solution (80 ml) was added, the resulting mixture stirred for 30 minutes and then DCM (400 ml) was added. The organic layer was separated and evaporated to give the title compound D10 (2.28 g) as a white solid. HPLC (walk-up): rt=3.56 min. ¹H NMR (400 MHz, DMSO-d₆) δ ppm: 8.31 (d, 1H), 7.86 (dd, 1H), 2.35 (s, 3H).

### Description 11

### 5-Fluoro-3-methyl-2-pyridinamine (D11)

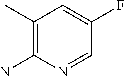

To a solution of 2-chloro-5-fluoro-3-methylpyridine D10 (0.50 g) in dry toluene (12.5 ml) were added sodium t-butoxyde (0.462 g, 4.81 mmol), Pd₂(dba)₃ (0.315 g, 0.344 mmol), BINAP (0.642 g, 1.031 mmol) and benzophenone imine (0.692 ml, 4.12 mmol). The resulting mixture was degassed (3×pump/N₂) and then heated to 80° C. After 1 hour stirring, the mixture was cooled down to room temperature, diluted with Et₂O (400 ml) and filtered through a celite pad. Volatiles were evaporated, the resulting oil was dissolved in THF (34 ml) and HCl (1.408 ml of a 2 M aqueous solution, 2.82 mmol) was added. The mixture was stirred at room temperature for 1.5 hours, then neutralized with a saturated NaHCO₃ aqueous solution and diluted with DCM (200 ml). The inorganic layer was back-extracted with DCM (2×50 ml). The collected organic layers were dried (Na₂SO₄), filtered and evaporated. The residue was purified by flash chromatography on silica gel (Biotage SP4 12M column, Cy/EtOAc 60/40). Collected fractions gave the title compound D11 (0.20 g) as an orange solid. MS: (ES/+) m/z: 127 (M+1). C₆H₇FN₂ requires 126. ¹H NMR (400 MHz, DMSO-d₆) δ ppm: 7.73 (d, 1H), 7.23 (dd, 1H), 5.60 (bs, 2H), 2.04 (s, 3H).

### Description 12

### 6-Fluoro-8-methyl-2-[(2S)-2-piperidinylmethyl]imidazo[1,2-a]pyridine (free base) (D12)

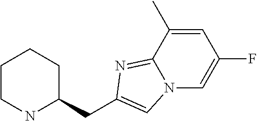

To a solution of 1,1-dimethylethyl (2S)-2-(3-bromo-2-oxopropyl)-1-piperidinecarboxylate D2 (0.15 g) in DMF (1 ml) was added 5-fluoro-3-methyl-2-pyridinamine D11 (0.0709 g) and the mixture was stirred at 80° C. for 1 hour. The reaction mixture was eluted through a SCX column. Collected fractions gave 0.137 g of an oil containing a mixture of the title compound, the corresponding N-Boc protected derivative and some residual 5-fluoro-3-methyl-2-pyridinamine [N-Boc derivative data. UPLC: rt=0.56 min, peak observed: 348 (M+1). C₁₉H₂₆FN₃O₂ requires 347]. The crude was dissolved in DCM (2 ml) and the resulting solution cooled to 0° C. TFA (0.40 ml) was added dropwise, the reaction left under stirring for 1 hour and then eluted through a SCX column. Collected fractions gave the title compound as a free base D12 (0.093 g) contaminated with 5-fluoro-3-methyl-2-pyridinamine. The material was used without further purification in the next step. MS: (ES/+) m/z: 248 (M+1). C₁₄H₁₈FN₃ requires 247.

### Description 13

### 6-Fluoro-8-methyl-2-[(2S)-2-piperidinylmethyl]imidazo[1,2-a]pyridine dihydrochloride (D13)

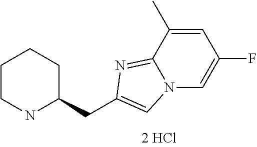

A mixture of 1,1-dimethylethyl (2S)-2-(3-bromo-2-oxopropyl)-1-piperidinecarboxylate D2 (0.94 g prepared by the method of D2 preparation (iii)), 5-fluoro-3-methyl-2-pyridinamine D11 (0.41 g) and NaHCO₃ (0.37 g, 4.40 mmol) in toluene (4.70 ml) was stirred at 90° C. overnight. The mixture was allowed to cool down to room temperature and the inorganic salts were removed by filtration. The solid cake was washed with toluene (2×0.94 ml). HCl 5-6 N solution in IPA (2.22 ml, 11.10-13.32 mmol) was added to 5.18 g of the toluene solution (filtrate, 5.46 g) of the free base D12. The mixture was heated to 70° C. and the resulting slurry stirred at that temperature under nitrogen atmosphere for 1 hour. The slurry was aged at 70° C. for 1 hour, cooled down to 40° C. over 2 hours, allowed to reach room temperature and then stirred at that temperature overnight. The slurry was cooled down to 0° C. and aged at that temperature for 1 hour. The solid was collected by filtration, washed with IPA (2×1.9 ml) and dried under vacuo at 40° C. for 4 hours to afford the title compound D13 (0.53 g). ¹H NMR (600 MHz, DMSO-d₆) δ ppm: 15.18 (bs, 1H), 9.21 (bs, 1H), 9.07 (bs, 1H), 8.99 (s, 1H), 8.14 (s, 1H), 7.83 (bs, 1H), 3.15-3.65 (m, 4H), 2.61 (s, 3H), 1.85 (d, 1H), 1.69-1.79 (m, 2H), 1.48-1.67 (m, 2H), 1.38-1.48 (m, 1H). HPLC (walk-up, 3 min method): rt=1.28 min.

### Description 14

### 2-Bromo-3,5-dimethylpyridine (D14)

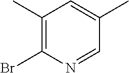

To a solution of DMAE (0.563 ml, 5.60 mmol) in hexane (5 ml) cooled to 0° C., was added dropwise BuLi 1.6 M in hexane (7.00 ml, 11.20 mmol). After 15 minutes, a solution of 3,5-dimethylpyridine (0.160 ml, 1.400 mmol) in hexane (5 ml) was added dropwise and the orange solution stirred for 1 hour at 0° C. After cooling at −78° C. a solution of CBr₄ (2.321 g, 7.00 mmol) in hexane (10 ml) was added dropwise. The reaction mixture was maintained at −78° C. for 0.5 hour then allowed to warm at room temperature. To the mixture was added at 0° C. water (25 ml) and the solution was extracted several times with Et₂O. The two phases were separated and the organic one was dried over Na₂SO₄. The solid was filtered out and the solvent was removed in vacuo. The crude was purified by flash chromatography on silica gel (Flash Master Personal, 50 g cartridge eluting from Cy 100% to Cy 90%:EtOAc 10%). The fractions were collected and the solvent removed in vacuo obtaining the title compound D14 (0.110 g). MS: (ES/+) m/z: 187 (M+1). C₇H₈BrN requires 186. ¹H NMR (400 MHz, CDCl₃) δ ppm: 8.03-8.04 (m, 1H), 7.36 (s, 1H), 2.37 (s, 3H), 2.28 (s, 3H).

### Description 15

### 3,5-Dimethyl-2-pyridinamine (D15)

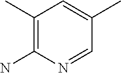

To a solution of 2-bromo-3,5-dimethylpyridine D14 (0.050 g) in dry Toluene (1 ml) were added sodium tertbutoxide (0.036 g, 0.376 mmol), Pd₂(dba)₃ (0.024 g, 0.027 mmol), BINAP (0.050 g, 0.081 mmol) and benzophenone immine (0.054 ml, 0.322 mmol). The resulting mixture was degassed (3×pump/N₂) then heated to 80° C. After 1.5 hours the mixture was cooled to room temperature, diluted with Et₂O (100 ml) and filtered through a celite pad. The solvents were evaporated. The resulting oil was dissolved with THF (20 ml) and HCl 2 M in water (0.269 ml, 0.537 mmol) was added and stirred at room temperature for 3 hours. The solution was concentrated in vacuo and the mixture was neutralized with saturated NaHCO₃ aqueous solution and DCM was added, the two layers were separated, the aqueous layer was extracted with DCM (3×100 ml). The collected organic layers were filtered through a phase separator and evaporated. The red oil obtained was purified by flash chromatography on silica gel (Flash Master personal, 10 g cartridge eluting first with Cy 80%: EtOAc 20%, and then with NH₃ 2 M in MeOH). The fractions were collected, the solvent was removed in vacuo obtaining the title compound D15 (0.022 g). MS: (ES/+) m/z: 123 (M+1). C₇H₁₀N₂ requires 122. ¹H NMR (400 MHz, CDCl₃) δ ppm: 7.77 (s, 1H), 7.15 (s, 1H), 4.5-4.30 (br.s, 2H), 2.19 (s, 3H), 2.13 (s, 3H).

### Description 16

### 1,1-Dimethylethyl (2S)-2-[(6,8-dimethylimidazo[1,2-a]pyridin-2-yl)methyl]-1-piperidinecarboxylate (D16)

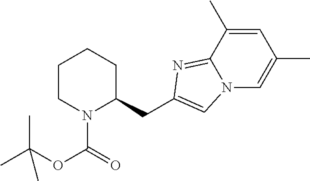

1,1-Dimethylethyl (2S)-2-(3-bromo-2-oxopropyl)-1-piperidinecarboxylate D2 (0.0629 g) and 3,5-Dimethyl-2-pyridinamine D15 (20 mg) were dissolved in DMF (1.5 ml) and heated at 70° C. for 3 hours. The solvent was removed and the crude was purified by flash chromatography (Sp4, 25M NH cartridge, eluting from Cy 100% to Cy 80%: EtOAc 20%). The fractions were collected and the solvent removed in order to obtain the title compound D16 (0.48 g). MS: (ES/+) m/z: 344 (M+1). C₂₀H₂₉N₃O₂ requires 343. ¹H NMR (400 MHz, CDCl₃) δ ppm: 7.68 (s, 1H), 7.30-7.25 (m, 1H), 6.78 (s, 1H), 4.78-4.53 (m, 1H), 4.21-3.88 (m, 1H), 3.27-3.09 (m, 1H), 3.04-2.79 (m, 2H), 2.57 (s, 3H), 2.25 (s, 3H), 1.78 (s, 9H), 1.75-1.03 (m, 6H)

### Description 17

### 6,8-Dimethyl-2-[(2S)-2-piperidinylmethyl]imidazo[1,2-a]pyridine (D17)

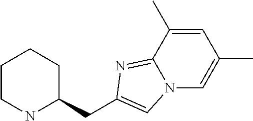

1,1-Dimethylethyl (2S)-2-[(6,8-dimethylimidazo[1,2-a]pyridin-2-yl)methyl]-1-piperidinecarboxylate D16 (0.046 g) was dissolved in DCM (4 ml) and to the solution TFA (1 ml) was added dropwise. The reaction was left stirring for 2 hours. The solvent was removed in vacuo and the residue was filtered through an SCX cartridge. The solvent was removed in vacuo obtaining the title compound D17 (0.026 g). MS: (ES/+) m/z: 244 (M+1). C₁₅H₂₁N₃ requires 243. ¹H NMR (400 MHz, CDCl₃) δ ppm: 7.73 (s, 1H), 7.32 (s, 1H), 6.80 (s, 1H), 3.09-2.59 (m, 5H), 2.57 (s, 3H), 2.28 (s, 3H), 1.83-1.21 (m, 6H).

### Description 18

### Bis(1,1-dimethylethyl) (3-bromo-4-methyl-2-pyridinyl)imidodicarbonate (D18)

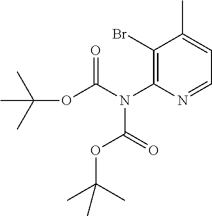

3-bromo-4-methyl-2-pyridinamine (1 g, 5.35 mmol) and BOC-Anhydride (3.72 ml, 16.04 mmol) in Tert-Butanol (6 ml) were heated at 35° C. After 3 hours to the solution was added DMAP (0.131 g, 1.069 mmol) and the reaction was left stirring overnight. The solvent was removed in vacuo and the crude purified by flash chromatography on silica gel (Flash Master Personal, 50 g cartridge, eluting with DCM 100%). The solvent was removed in vacuo obtaining the title compound D18 (1.5 g). MS: (ES/+) m/z: 389 (M+1). C₁₆H₂₃BrN₂O₄ requires 387.

¹H NMR (400 MHz, CDCl₃) δ ppm: 8.32-8.31 (d, 2H), 7.16-7.15 (d, 2H), 2.49 (s, 3H), 2.17 (s, 3H), 1.43 (s, 18H).

### Description 19

### Bis(1,1-dimethylethyl) (3,4-dimethyl-2-pyridinyl)imidodicarbonate (D19)

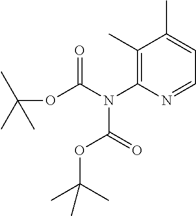

Bis(1,1-dimethylethyl) (3-bromo-4-methyl-2-pyridinyl)imidodicarbonate D18 (0.600 g) was dissolved in DME (1.5 ml). To the solution were added methyl boronic acid (0.100 g, 1.67 mmol), Tetrakis(triphenylphosphine)Palladium(0) (0.090 g, 0.077 mmol) and Cs₂CO₃ (1.514 g, 4.65 mmol). The mixture was heated at 90° C. in the Microwave (5×20 minutes). To the mixture were added more methyl boronic acid (0.060 g, 1.00 mmol) and Tetrakis(triphenylphosphine)Palladium(0) (0.030 g, 0.025 mmol) and the suspension was heated in the Microwave at 90° C. (3×20 minutes). The mixture was filtered and the solvent removed in vacuo. The crude was purified by Flash Chromatography (Sp4, 25M cartridge eluting from Cy 100% to Cy 80%: EtOAc 20%). The fractions were collected and the solvent was removed in vacuo obtaining the title compound D19 (0.255 g). MS: (ES/+) m/z: 323 (M+1). C₁₇H₂₆N₂O₄ requires 322. ¹H NMR (400 MHz, CDCl₃) δ ppm: 8.24-8.23 (d, 2H), 7.08-7.07 (d, 2H), 2.35 (s, 3H), 2.17 (s, 3H), 1.44 (s, 18H).

### Description 20

### 3,4-Dimethyl-2-pyridinamine (D20)

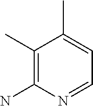

Bis(1,1-dimethylethyl) (3,4-dimethyl-2-pyridinyl)imidodicarbonate D19 (0.0255 g) was dissolved in DCM (6 ml) and TFA (1 ml) was added at 0° C. The reaction was left stirring for 4 hours at room temperature. The solvent was removed in vacuo and the residue was filtered through an SCX cartridge. The solvent was removed in vacuo obtaining the title compound D20 (0.088 mg). MS: (ES/+) m/z: 123 (M+1). C₇H₁₀N₂ requires 122. ¹H NMR (400 MHz, CDCl₃) δ ppm: 7.82-7.81 (d, 1H), 6.56-6.53 (d, 1H), 4.62-4.44 (m, 2H), 2.26 (s, 3H), 2.08 (s, 3H).

### Description 21

### 1,1-dimethylethyl (2S)-2-[(7,8-dimethylimidazo[1,2-a]pyridin-2-yl)methyl]-1-piperidinecarboxylate (D21)

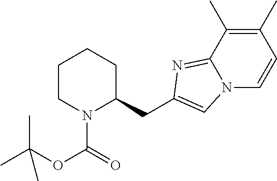

1,1-Dimethylethyl (2S)-2-(3-bromo-2-oxopropyl)-1-piperidinecarboxylate D2 (0.0577 g) and 3,4-Dimethyl-2-pyridinamine D20 (0.020 g) were dissolved in DMF (1.5 ml) and heated at 70° C. for 3 hours. The solvent was removed in vacuo and the crude purified by flash chromatography (SP4, 25M NH cartridge eluting from Cy 100% to Cy 80%: EtOAc 20%). The fractions were collected and the solvent removed obtaining the title compound 921 (0.0499 g). MS: (ES/+) m/z: 344 (M+1). C₂₀H₂₉N₃O₂ requires 343. ¹H NMR (400 MHz, CDCl₃) δ ppm: 7.81-7.76 (d, 1H), 7.34-7.27 (m, 1H), 6.56-6.51 (d, 1H), 4.75-4.59 (m, 1H), 4.17-3.95 (m, 1H), 3.25-3.17 (m, 1H), 3.03-2.79 (m, 2H), 2.54 (s, 3H), 2.33 (s, 3H), 1.86-1.12 (m, 15H).

### Description 22

### 7,8-dimethyl-2-[(2S)-2-piperidinylmethyl]imidazo[1,2-a]pyridine (D22)

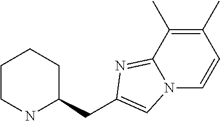

1,1-Dimethylethyl (2S)-2-[(7,8-dimethylimidazo[1,2-a]pyridin-2-yl)methyl]-1-piperidinecarboxylate D21 (0.0499 g) was dissolved in DCM (4 ml) and TFA (1 ml) was added dropwise. The reaction was left stirring 2 hours. The solvent was removed in vacuo and the residue was filtered through an SCX cartridge obtaining the title compound D22 (0.033 mg). MS: (ES/+) m/z: 244 (M+1). C₁₅H₂₁N₃ requires 243. ¹H NMR (400 MHz, CDCl₃) δ ppm: 7.85-7.80 (d, 1H), 7.34-7.31 (s, 1H), 6.59-6.54 (d, 1H), 3.11-2.87 (m, 3H), 2.78-2.70 (m, 1H), 2.69-2.59 (m, 1H), 2.54 (s, 3H), 2.33 (s, 3H), 1.86-1.21 (m, 6 H).

### Description 23

### Bis(1,1-dimethylethyl) (5-bromo-4-methyl-2-pyridinyl)imidodicarbonate (D23)

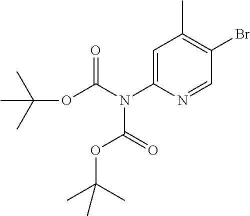

5-Bromo-4-methyl-2-pyridinamine (0.300 g, 1.604 mmol), BOC-Anhydride (0.819 ml, 3.53 mmol) and DMAP (0.0392 g, 0.321 mmol) in Tert-Butanol (4 ml) were heated at 35° C. overnight. The solvent was removed in vacuo and the crude was purified by flash chromatography on silica gel (Flash Master personal, 50 g cartridge, eluting with DCM 100%). The solvent was removed in vacuo obtaining 1,1-dimethylethyl (5-bromo-4-methyl-2-pyridinyl)carbamate (0.200 g) and the title compound D23 (0.300 g, 0.775 mmol, 48.3% yield). MS: (ES/+) m/z: 388 (M+1). C₁₆H₂₃BrN₂O₄ requires 387.27. ¹H NMR (400 MHz, CDCl₃) δ ppm: 8.52 (s, 1H), 7.17 (s, 1H), 2.43 (s, 3H), 1.49 (s, 18H).

### Description 24

### 1,1-Dimethylethyl (4,5-dimethyl-2-pyridinyl)carbamate (D24)

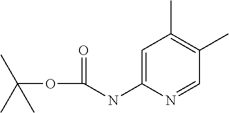

To a degassed solution of bis(1,1-dimethylethyl) (5-bromo-4-methyl-2-pyridinyl)imidodicarbonate D23 (0.100 g) in 1,4-Dioxane (2 ml), Pd₂(dba)₃ (0.0118 g, 0.013 mmol), tricyclohexylphosphyne (0.015 g, 0.053 mmol), trimethylboroxine (0.054 ml, 0.387 mmol) and Cs₂CO₃ (0.252 g, 0.775 mmol) were added. The mixture was heated at 80° C. in the Microwave for 15 minutes. The mixture was filtered on a celite pad, to the residue were added H₂O and DCM, the aqueous phase was washed several times with DCM. The two phases were separated and the organic one filtered through a phase separator obtaining the title compound D24 (0.060 g) as crude. MS: (ES/+) m/z: 223 (M+1). C₁₂H₁₈N₂O₂ requires 222. ¹H NMR (400 MHz, CDCl₃) δ ppm: 7.79 (s, 1H), 7.76 (s, 1H), 2.28 (s, 3H), 2.19 (s, 3H), 1.55 (s, 9H).

### Description 25

### 4,5-Dimethyl-2-pyridinamine (D25)

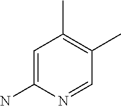

1,1-Dimethylethyl (4,5-dimethyl-2-pyridinyl)carbamate D24 (0.090 g) was dissolved in DCM (5 ml), to the solution was added TFA (1 ml) and the reaction was left stirring for 1.5 hours. The solvent was removed in vacuo and the residue was filtered through an SCX cartridge (10 g). The solution was concentrated under vacuum obtaining the title compound D25 (0.0375 g). MS: (ES/+) m/z: 123 (M+1). C₇H₁₀N₂ requires 122.

¹H NMR (400 MHz, CDCl₃) δ ppm: 2.12 (s, 3H) 2.19 (s, 3H) 4.51-3.94 (br. s., 2H) 6.32-6.39 (m, 1H) 7.80 (s, 1H).

### Description 26

### 1,1-Dimethylethyl (2S)-2-[(6,7-dimethylimidazo[1,2-a]pyridin-2-yl)methyl]-1-piperidinecarboxylate (D26)

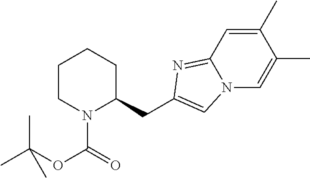

4,5-Dimethyl-2-pyridinamine D25 (0.0375 g) and 1,1-dimethylethyl (2S)-2-(3-bromo-2-oxopropyl)-1-piperidinecarboxylate D2 (0.108 g) were dissolved in DMF (2 ml) and heated at 75° C. for 2 hours. The solvent was removed in vacuo and the residue was purified by flash chromatography (Sp4 25 g NH cartridge eluting from Cy 100% to Cy 80%:EtOAc 20%). The fractions were collected and the solvent removed in vacuo obtaining the title compound D26 (0.050 g). MS: (ES/+) m/z: 344 (M+1). C₂₀H₂₉N₃O₂ requires 343. ¹H NMR (400 MHz, CDCl₃) δ ppm: 0.75-1.53 (m, 15H), 2.22 (s, 3H), 2.34 (s, 3H), 2.78-3.00 (m, 2H), 3.04-3.19 (m, 1H), 3.88-4.17 (m, 1H), 4.51-4.72 (m, 1H), 7.25-7.30 (m, 2H), 7.78 (s, 1H).

### Description 27

### 6,7-Dimethyl-2-[(2S)-2-piperidinylmethyl]imidazo[1,2-a]pyridine (D27)

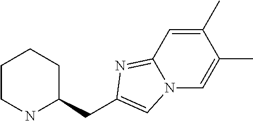

1,1-Dimethylethyl (2S)-2-[(6,7-dimethylimidazo[1,2-a]pyridin-2-yl)methyl]-1-piperidinecarboxylate D26 (0.050 g) was dissolved in DCM (4 ml), to the solution was added drop wise TFA (1 ml) and the solution was left stirring 2 hours at room temperature. The solvent was removed in vacuo and the residue was filtered through an SCX cartridge, eluting first with MeOH and then with NH₃ in MeOH 2N, the fractions were collected and the solvent evaporated obtaining the title compound D27 (0.026 g). MS: (ES/+) m/z: 244 (M+1). C₁₅H₂₁N₃ requires 243. ¹H NMR (400 MHz, CDCl₃) δ ppm: 7.81 (s, 1H), 7.30-7.24 (m, 2H), 3.07-2.57 (m, 5H), 2.32 (s, 3H), 2.23 (s, 3H), 1.90-1.15 (m, 6H)

### Description 28

### 1,1-Dimethylethyl (2S)-2-[(3-chloro-8-methylimidazo[1,2-a]pyridin-2-yl)methyl]-1-piperidinecarboxylate (D28)

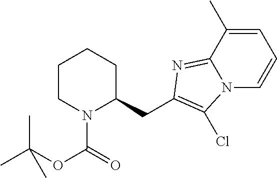

To a solution of 1,1-dimethylethyl (2S)-2-[(8-methylimidazo[1,2-c]pyridin-2-yl)methyl]-1-piperidinecarboxylate D3 (0.18 g) in DCM (4 ml) was added NCS (0.082 g, 0.62 mmol) and the reaction mixture was stirred at room temperature for 30 minutes. The solvent was evaporated to afford the title compound D28 (0.29 g) as a crude material which was used in the next step without any further purification. MS: (ES/+) m/z: 364 (M+1). C₁₉H₂₆ClN₃O₂ requires 363.

### Description 29

### 3-Chloro-8-methyl-2-[(2S)-2-piperidinylmethyl]imidazo[1,2-a]pyridine (D29)

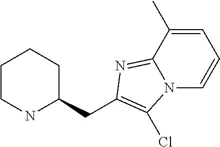

To a solution of 1,1-dimethylethyl (2S)-2-[3-chloro-8-methylimidazo[1,2-c]pyridin-2-yl)methyl]-1-piperidinecarboxylate D28 (0.29 g) in DCM (6 ml), TFA (1.20 ml) was added dropwise at 0° C. and the reaction mixture was stirred for 1 hour. The solvent was evaporated and the residue eluted through a SCX column. Collected fractions gave the title compound D29 (0.17 g) as a crude material which was used in the next step without any further purification. MS: (ES/+) m/z: 264 (M+1). C₁₄H₁₈ClN₃ requires 263. HPLC (walk-up): rt=2.20 min.

### Description 30

### 2-(Hydroxymethyl)-6-methyl-4-nitro-3-pyridinol (D30)

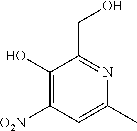

A cooled mixture of 70% HNO₃ (0.459 ml) and H₂SO₄ (0.575 ml) was added dropwise to an ice-cooled solution of 2-(hydroxymethyl)-6-methyl-3-pyridinol (available from Sigma-Aldrich #144428) (1 g, 7.19 mmol) in concentrated H₂SO₄ (4.5 ml). The mixture was allowed to reach room temperature and stirred for 4 hours. Presence of starting material was shown by UPLC/MS: MS: (ES/+) m/z: 140 (M+1). The reaction mixture was cooled again to 0-5° C. and was added a mixture of 70% HNO₃ (0.918 ml) and H₂SO₄ (1.149 ml), the resulting mixture was warmed to room temperature and stirred for 2 hours. An additional amount (2 ml) of the 70% HNO₃ and H₂SO₄ mixture with the previous ratio (1:1.5) was added and stirred for 1 hour at room temperature. The reaction mixture was cooled to 0° C. and was added dropwise NH₄OH until pH˜5 and then was extracted with DCM, separated through a phase separator cartridge and evaporated under reduced pressure. The brown oil was purified by flash chromatography on silica gel (Biotage SP4, 25+M column, eluted with 10 volumes of DCM/MeOH, 49/1). The title compound D30 (0.290 g) and 6-methyl-2-nitro-3-pyridinol (0.330 g, 2.120 mmol, 29.5% yield) were recovered. HPLC (walk-up): rt=1.86 min. C₇H₈N₂O₄ requires 184. ¹H NMR (400 MHz, DMSO-d₆) δ ppm: 11.29-9.78 (br.s, 1H), 7.62 (s, 1H), 5.84-4.92 (br.s, 1H), 4.66 (s, 2H), 2.47-2.44 (s, 3H).

### Description 31

### [3-(Ethyloxy)-6-methyl-2-pyridinyl]methanol (D31)

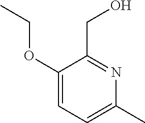

2-(hydroxymethyl)-6-methyl-3-pyridinol (available from Sigma-Aldrich #144428) (1.5 g, 10.78 mmol), K₂CO₃ (7.45 g, 53.9 mmol) and iodoethane (1.724 ml, 21.56 mmol) were dissolved in DMF (15 ml). The mixture was left stirring at room temperature overnight. To the solution were added H₂O and EtOAc. The two layers were separated. The aqueous one was extracted several times with EtOAc. The combinated organic layers were washed with brine/ice and dried over Na₂SO₄. The solid was filtered out and the solvent was removed in vacuo to afford the title compound D31 (1.669 g) as a pale yellow solid. ¹H-NMR (400 MHz, CDCl₃) δ ppm: 6.98-7.06 (m, 2H), 4.72 (s, 2H), 4.47 (bs, 1H), 4.05 (q, 2H), 2.50 (s, 3H), 1.43 (t, 3H).

The following compounds of formula (A) were prepared using a similar procedure to that described above for Description 31. Each compound was obtained by O-alkylation of 2-(hydroxymethyl)-6-methyl-3-pyridinol or 2-(Hydroxymethyl)-6-methyl-4-nitro-3-pyridinol D30 and a suitable electrophile. This is provided merely for assistance to the skilled chemist. The starting material may not necessarily have been prepared from the batch referred to.

A

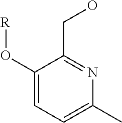

| No. | Structure | Characterising data |
|---|---|---|
| D32 | 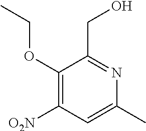 | [3-(ethyloxy)-6-methyl-4-nitro-2-pyridinyl]methanol HPLC (walk-up): rt = 3.40 min. MS: (ES/+) m/z: 213 (M + 1) C₉H₁₂N₂O₄ requires 212. ¹H NMR (400 MHz, DMSO-d₆) δ ppm: 7.71 (s, 1 H), 5.34 (t, 1 H), 4.60 (d, 2 H), 4.07 (q, 2 H), 1.27- 1.34 (m, 3 H). |
| D33 | 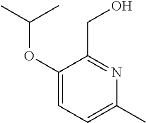 | {6-methyl-3-[(1-methylethyl)oxy]-2- pyridinyl}methanol MS: (ES/+) m/z: 182 (M + 1). C₁₀H₁₅NO₂ requires 181. ¹H-NMR (400 MHz, CDC1₃) δ ppm: 7.07 (d, 1 H), 7.00 (d, 1 H), 4.70 (s, 2 H), 4.46-4.56 (m, 2 H), 2.50 (s, 3 H), 1.35 (s, 3 H), 1.34 (s, 3 H). |
| D34 | 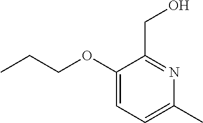 | [6-methyl-3-(propyloxy)-2-pyridinyl]methanol MS: (ES/+) m/z: 182 (M + 1). C₁₀H₁₅NO₂ requires 181. ¹H-NMR (400 MHz, CDCl₃) δ ppm: 7.07-6.98 (m, 2 H), 4.73 (s, 2 H), 4.50 (bs, 1 H), 3.94 (t, 2 H), 2.51 (s, 3 H), 1.83 (sest, 2 H), 1.05 (t, 3 H). |
| D35 | 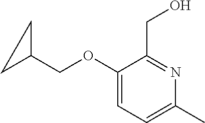 | {3-[(cyclopropylmethyl)oxy]-6-methyl-2- pyridinyl} methanol MS: (ES/+) m/z: 194 (M + 1). C₁₁H₁₅NO₂ requires 193. ¹H-NMR (400 MHz, CDCl₃) δ ppm: 7.02 (m, 2 H), 4.75 (s, 2 H), 4.48 (bs, 1 H), 3.79-3.88 (m, 2 H), 2.51 (s, 3 H), 1.18-1.33 (m, 1 H), 0.58-0.71- (m, 2 H), 0.29-0.41 (m, 2 H). |
| D36 | 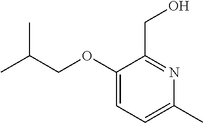 | {6-methyl-3-[(2-methylpropyl)oxy]-2- pyridinyl} methanol MS: (ES/+) m/z: 196 (M + 1). C₁₁H₁₇NO₂ requires 195. ¹H-NMR (400 MHz, CDCl₃) δ ppm: 6.97-7.05 (m, 2 H), 4.74 (d, 2 H), 4.49 (bt, 1 H), 3.71-3.77 (m, 2 H), 2.51 (s, 3 H), 2.18-2.05 (m, 1 H), 1.05 (s, 3 H), 1.04 (s, 3 H). |

### Description 37

### 3-(Ethyloxy)-6-methyl-2-pyridinecarboxylic acid (D37)

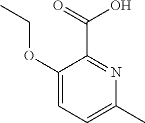

[3-(ethyloxy)-6-methyl-2-pyridinyl]methanol D31 (1.67 g, crude the of material obtained in the Description 31) in acetonitrile (50 ml) and phosphate buffer (38.0 ml) was added TEMPO (0.218 g, 1.397 mmol) at room temperature. After warming to 35° C. a solution of NaClO₂ (4.51 g, 49.9 mmol) in water (10 ml) and a solution of NaClO (18.96 ml, 39.9 mmol) were added simultaneously over 1 hour. After stirring 4 hours at 35° C., water (40 ml) was added to the reaction mixture which was then adjusted to pH 8 by addition of 1 M NaOH. The mixture was poured into ice-cold saturated aqueous sodium thiosulfate solution (100 ml) and stirring was continued for 30 minutes. The pH was adjusted to pH 3 by slow addiction of 1 M HCl and the aqueous phase was extracted with DCM (6×200 ml). The combined organic layers were washed with brine (2×200 ml), dried over Na₂SO₄ and concentrated to afford the title compound D37 (1.64 g). MS: (ES/+) m/z: 182 (M+1). C9H₁₁NO₃ requires 181. ¹H-NMR (400 MHz, DMSO d⁶) δ ppm: 12.90 (bs, 1H), 7.49 (d, 1 H), 7.31 (d, 1H), 4.08 (q, 2H), 2.40 (s, 3H), 1.29 (t, 3H).

The following compounds of formula (B) were prepared using a similar procedure to that described above for Description 37. Each compound was obtained by the primary alcohol oxidation of the corresponding 2-(alkoxy)-6-methyl-3-pyridinol derivative. This is provided merely for assistance to the skilled chemist. The starting material may not necessarily have been prepared from the batch referred to.

B

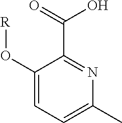

| No. | Structure | Characterising data |
|---|---|---|
| D38 | 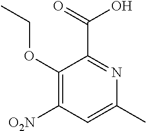 | 3-(ethyloxy)-6-methyl-4-nitro-2- pyridinecarboxylic acid HPLC (walk-up): rt = 3.29 min. MS: (ES/−) m/z: 225 (M − 1). C₉H₁₀N₂O₅ requires 226. ¹H NMR (400 MHz, DMSO-d₆) δ ppm: 7.66 (s, 1 H), 4.06-4.24 (m, 2 H), 2.45-2.49 (m, 3 H), 1.22 (m, 3 H) |
| D39 | 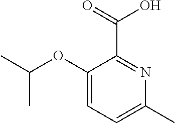 | 6-methyl-3-[(1-methylethyl)oxy]-2- pyridinecarboxylic acid MS: (ES/+) m/z: 196 (M + 1). C₁₀H₁₃NO₃ requires 195. ¹H-NMR (400 MHz, DMSO d⁶) δ ppm: 12.90 (bs, 1 H), 7.49 (d, 1 H), 7.29 (d, 1 H), 4.61 (hept, 1 H), 2.39 (s, 3 H), 1.24 (d, 6 H). |
| D40 | 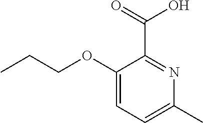 | 6-methyl-3- [(1-methylethyl)oxy] -2- pyridinecarboxylic acid HPLC: rt = 1.86 min. MS: (ES/+) m/z: 196 (M + 1). C₁₀H₁₃NO₃ requires 195. ¹H-NMR (400 MHz, DMSO d⁶) δ ppm: 12.82-13.04 (bs, 1 H), 7.46-7.51 (d, 1 H), 7.29-7.31 (d, 1 H), 3.95-4.02 (m, 2 H), 2.51 (s, 3 H), 1.62-1.75 (m, 2 H), 0.90-1.00 (m, 3 H). |
| D41 | 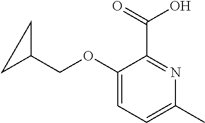 | 3-[(cyclopropylmethyl)oxy]-6-methyl-2- pyridinecarboxylic acid MS: (ES/+) m/z: 208 (M + 1). C₁₁H₁₃NO₃ requires 207. ¹H-NMR (400 MHz, CDCl₃) δ ppm: 7.34-7.43 (m, 2 H), 3.98-4.06 (m, 2 H), 2.53 (s, 3 H), 1.28-1.43 (m, 1 H), 0.58-0.75 (m, 2 H), 0.37-0.52 (m, 2 H). |
| D42 | 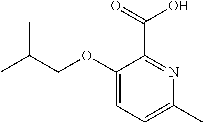 | 6-methyl-3-[(2-methylpropyl)oxy]-2- pyridinecarboxylic acid MS: (ES/+) m/z: 210 (M + 1). C₁₁H₁₅NO₃ requires 209. ¹H-NMR (400 MHz, CDCl₃) δ ppm: 7.38 (s, 2 H), 3.89 (d, 2 H), 2.57 (s, 3 H), 2.15-2.30 (sext, 1 H), 1.08-1.13 (m, 6 H). |

### Description 43

### 4-Chloro-3-(ethyloxy)-6-methyl-2-pyridinecarboxylic acid (D43)

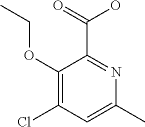

To a solution of 3-(ethyloxy)-6-methyl-4-nitro-2-pyridinecarboxylic acid D38 (0.280 g) in DCM (2 ml), DMF (2 μl, 0.026 mmol) and oxalyl chloride (0.130 ml, 1.485 mmol) were added and the resulting mixture was stirred for 1 hour at room temperature. A small sample was withdrawn and diluted with anhydrous MeOH: MS showed complete conversion to the methyl ester. MeOH (0.250 ml, 6.19 mmol) was added dropwise to the reaction mixture and stirred for 30 minutes. To the reaction was added DCM (2 ml) and saturated Na₂CO₃ aqueous solution (2 ml) and the aqueous layer was extracted with DCM (2×2 ml). The organic phase was dried through a phase separator cartridge and evaporated to obtain methyl 4-chloro-3-(ethyloxy)-6-methyl-2-pyridinecarboxylate (0.107 g). The product was recovered impure and used with no further purification. MS: (ES/+) m/z: 230 (M+1). MS: (ES/+) m/z: 230 (M+1). C₁₀H₁₂ClNO₃ requires 229.

Methyl 4-chloro-3-(ethyloxy)-6-methyl-2-pyridinecarboxylate (0.107 g) was dissolved in THF (4 ml), MeOH (1.000 ml), water (1.000 ml) and LiOH H₂O (0.0176 g, 0.419 mmol) was added. The resulting mixture was left stirring at room temperature 2 hours. To the solution was added HCl 1 M until pH 3, and the mixture was extracted several times with EtOAc. The organic layer was dried over Na₂SO₄, and the solvent was removed under reduced pressure in order to obtain the title compound D43 (0.109 g), like brown semisolid. HPLC (walk-up): rt=2.71 min. MS: (ES/+) m/z: 216 (M+1). C₉H₁₀ClNO₃ requires 215. ¹H NMR (400 MHz, DMSO-d₆) δ ppm: 13.53 (br. s., 1H), 7.62 (d, 1H), 4.06 (m, 2H), 2.44 (s, 3H), 1.27-1.35 (m, 3H).

### Description 44

### Methyl 3-hydroxy-2-pyridinecarboxylate (D44)

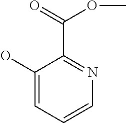

3-hydroxy-2-pyridinecarboxylic acid (1 g, 7.19 mmol) was dissolved in DCM (20 ml), to the solution under N₂ atmosphere oxalyl chloride (1.510 ml, 17.25 mmol) was added dropwise and the reaction was left stirring for 1 hour. After that time MeOH (2 ml, 49.4 mmol) was added and left stirring at room temperature for 2 hours more. The solvent was removed in vacuo and the residue redissolved in DCM and washed with saturated NaHCO₃ aqueous solution. The two phases were separated and the organic one was filtered through a phase separator and evaporated. The crude was purified by flash chromatography on silica gel (Flash Master Personal 50 g cartridge eluting with Cy 80%: EtOAc 20%). The fractions were collected and the solvent removed in vacuo obtaining the title compound D44 (0.800 g) as a white solid. MS: (ES/+) m/z: 154 (M+1). C₇H₇NO₃ requires 153. ¹H-NMR (400 MHz, CDCl₃) δ ppm: 10.66 (s, 1H), 8.32-8.29 (m, 1H), 7.48-7.43 (m, 2H), 4.09 (s, 3 H).

### Description 45

### Methyl 6-Bromo-3-hydroxy-2-pyridinecarboxylate (D45)

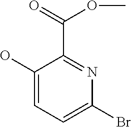

To a stirred solution of methyl 3-hydroxy-2-pyridinecarboxylate D44 (0.100 g) in water (5 ml), Br₂ (0.045 ml, 0.882 mmol) was added dropwise and the solution was left stirring at room temperature. A precipitate occurred. The mixture was left stirring for 30 minutes, then DCM was added and the two phases were separated. The aqueous one was extracted with DCM. The organic layers were filtered through a phase separator and evaporated. The crude was purified by flash chromatography on silica gel (Flash Master Personal, 10 g cartridge eluting with Cy 90% EtOAc 10%). The fractions were collected and the solvent removed in vacuo obtaining the title compound D45 (0.100 g). MS: (ES/+) m/z: 233 (M+1). C₇H₆BrNO₃ requires 232. ¹H-NMR (400 MHz, CDCl₃) δ ppm: 10.69 (s, 1H), 7.61-7.51 (d, 1H), 7.32-7.25 (d, 1H), 4.06 (s, 3H).

### Description 46

### Methyl 6-bromo-3-(ethyloxy)-2-pyridinecarboxylate (D46)

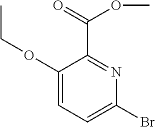

Methyl 6-bromo-3-hydroxy-2-pyridinecarboxylate D45 (0.200 g, 0.862 mmol), K₂CO₃ (0.596 g, 4.31 mmol) and iodoethane (0.139 ml, 1.724 mmol) were dissolved DMF (3 ml). The mixture was left stirring at room temperature overnight. To the solution were added H₂O and DCM. The two layers were separated. The aqueous one was extracted several times with DCM. The organic layers were washed with brine/ice, filtered through a phase separator and evaporated. The crude was purified by flash chromatography on silica gel (Flash Master Personal, 20 g cartridge eluting from Cy 100% to Cy 90%: EtOAc 10%). The fractions were collected obtaining the title compound D46 (0.200 g). MS: (ES/+) m/z: 260 (M+1). C₉H₁₀BrNO₃ requires 259. ¹H-NMR (400 MHz, CDCl₃) δ ppm: 7.55-7.53 (d, 1 H), 7.25-7.23 (d, 1H), 4.18-4.10 (m, 2H), 3.96 (s, 3H), 1.50-1.43 (m, 3H).

### Description 47

### Methyl 6-ethenyl-3-(ethyloxy)-2-pyridinecarboxylate (D47)

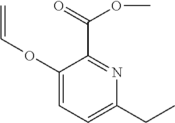

Methyl 6-bromo-3-(ethyloxy)-2-pyridinecarboxylate D46 (0.200 g) was dissolved in DMF (3 ml), to the solution was added vinyltri-N-butyltin (0.271 ml, 0.923 mmol) and it was degassed by bubbling nitrogen through it for 30 minutes, then Tetrakis(triphenylphosphine)Palladium(0) (0.089 g, 0.077 mmol) was added and the mixture was heated at 95° C. (3×20 minutes) in the Microwave. The mixture was filtered through a celite pad and the residue was purified by Flash Chromatography (Sp4 25M cartridge eluting from Cy 100% to Cy 80%:EtOAc 20%). The fractions were collected and the solvent evaporated obtaining the title compound D47 (0.119 g). HPLC (walk up): rt=3.95 min. C₁₁H₁₃NO₃ requires 207. ¹H-NMR (400 MHz, CDCl₃) δ ppm: 7.52-7.48 (d, 1H), 7.33-7.28 (m, 1H), 6.88-6.77 (m, 1H), 6.08-6.0 (m, 1H), 5.48-5.39 (m, 1H), 4.19-4.15 (m, 2H), 3.99 (s, 3H), 1.51-1.43 (m, 3H).

### Description 48

### Methyl 6-ethyl-3-(ethyloxy)-2-pyridinecarboxylate (D48)

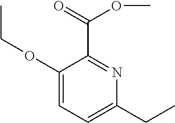

To a solution of methyl 6-ethenyl-3-(ethyloxy)-2-pyridinecarboxylate D47 (0.119 g) in EtOH (5 ml), PtO₂ (0.013 g, 0.057 mmol) was added and the mixture was left to react under H₂ at 1 atmosphere for 15 minutes at room temperature. The suspension was filtered through a celite pad and the solvent was removed in vacuo obtaining the title compound D48 (0.109 g, 0.519 mmol, 90% yield). HPLC (walk up): rt=3.68 min. MS: (ES/+) m/z: 210 (M+1). C₁₁H₁₅NO₃ requires 209. ¹H-NMR (400 MHz, CDCl₃) δ ppm: 7.30-7.27 (m, 2 H), 4.15-4.0 (m, 2H), 3.98 (s, 3H), 2.87-2.80 (m, 2H), 1.48-1.43 (m, 2H), 1.32-1.27 (m, 3H).

### Description 49

### 6-Ethyl-3-(ethyloxy)-2-pyridinecarboxylic acid (D49)

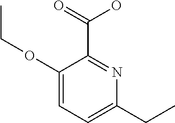

Methyl 6-ethyl-3-(ethyloxy)-2-pyridinecarboxylate D48 (0.109 g) was dissolved in THF (3 ml)/MeOH (0.750 ml)/Water (0.750 ml) to the solution was added LiOH (0.0374 g, 1.563 mmol) and the mixture was left stirring at room temperature for 2 hours. To the solution was added HCl 1 M until pH=3, and the mixture was extracted several times with DCM, it was filtered through a phase separator and the organic solvent was removed in vacuo obtaining the title compound D49 (0.081 g). HPLC (walk up): rt=2.12 min. MS: (ES/+) m/z: 196 (M+1). C₁₀H₁₃NO₃ requires 195. ¹H NMR (400 MHz, DMSO-d₆) δ ppm: 13.1-12.74 (br.s, 1H), 7.55-7.46 (m, 1H), 7.35-7.27 (m, 1H), 4.14-4.01 (m, 2H), 2.74-2.60 (m, 2H), 1.38-1.24 (m, 3H), 1.24-1.14 (m, 3H).

### Description 50

### 2-({[(1,1-Dimethylethyl)(dimethyl)silyl]oxy}methyl)-6-methyl-3-pyridinol (D50)

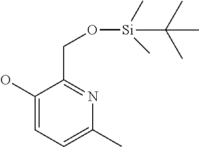

Imidazole (7.71 g, 113 mmol) and tert-butyldimethylsilyl chloride (6.82 g, 45.3 mmol) were added to a solution of 2-(hydroxymethyl)-6-methyl-3-pyridinol (5.25 g, 37.7 mmol) in anhydrous DMF (150 ml) with stirring at room temperature. The mixture was then stirred at 60° C. under nitrogen overnight. The mixture was diluted with DCM and washed with NH₄Cl and brine. The organic layer was evaporated and dried over Na₂SO₄. The residual material was purified by flash chromatography on silica gel (SP1, 40 M column, with Cy/EtOAc: from Cy 100 to Cy/EtOAc 90/10 elution) to afford the title compound D50 (5.52 g) as a white solid. MS: (ES/+) m/z: 254 (M+1). C₁₃H₂₃NO₂Si requires 253. ¹H NMR (400 MHz, DMSO-d₆) δ ppm: 9.5 (s, 1H), 7.03-7.06 (m, 1H), 6.95-6.98 (m, 1H), 4.67 (s, 2 H) 2.33 (s, 3H), 0.87-0.85 (m, 9H), 0.06-0.04 (m, 6H).

### Description 51

### 2-({[(1,1-Dimethylethyl)(dimethyl)silyl]oxy}methyl)-6-methyl-3-pyridinyl trifluoromethanesulfonate (D51)

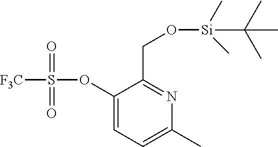

To a solution of 2-({[(1,1-dimethylethyl)(dimethyl)silyl]oxy}methyl)-6-methyl-3-pyridinol D50 (0.52 g) in anhydrous DCM (10 ml) was added DIPEA (1.075 ml, 6.16 mmol) dropwise with stirring. The mixture was then cooled to 0° C. and Triflic Anhydride (0.520 ml, 3.08 mmol) was added dropwise with stirring. The solution was allowed to warm up to room temperature and stirred under nitrogen for 4 hours. The solution was diluted with DCM (10 ml) and washed with water (2×20 ml). The organic layer was then dried over Na₂SO₄ and evaporated. The residual brown oil was purified by flash chromatography on silica gel (Companion, 120 g cartridge, with Cy/EtOAc: from Cy 100 to Cy/EtOAc 80/20 elution) to afford the title compound D51 (0.62 g) as a yellow oil. MS: (ES/+) m/z: 386 (M+1). C₁₄H₂₂F₃NO₄SSi requires 385. ¹H NMR (400 MHz, DMSO-d₆) δ ppm: 7.85-7.78 (d, 1H), 7.45-7.43 (d, 1H), 4.79 (s, 2H) 2.53-2.49 (m, 3H), 0.87-0.85 (m, 9H), 0.06-0.04 (m, 6H).

### Description 52

### 2-({[(1,1-Dimethylethyl)(dimethyl)silyl]oxy}methyl)-6-methyl-3-phenylpyridine (D52)

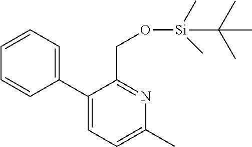

Nitrogen was passed through a suspension of 2-({[(1,1-dimethylethyl)(dimethyl)silyl]oxy}methyl)-6-methyl-3-pyridinyl trifluoromethanesulfonate D51 (0.200 g), phenyl boronic acid (0.127 g, 1.038 mmol) and anhydrous K₂CO₃ (0.108 g, 0.778 mmol) in Toluene (5 ml) for 15 minutes. Tetrakis(triphenylphosphine)Palladium(0) (0.060 g, 0.052 mmol) was added and the mixture was heated at 85-90° C. for 5 hours. The reaction mixture was cooled to 25° C., diluted with EtOAc (5 ml) and washed sequentially with saturated NaHCO₃ aqueous solution, NH₄Cl, water and brine. The organic phase was concentrated and the residue was purified by flash chromatography on silica gel (Companion, 80 g cartridge, with Cy/EtOAc from Cy 100 to Cy/EtOAc 80/20 elution) to afford the title compound D52 (0.114 g) as a yellow oil. MS: (ES/+) m/z: 314 (M+1). C₁₉H₂₇NOSi requires 313. ¹H NMR (400 MHz, DMSO-d₆) δ ppm 7.59 (d, 1H), 7.35-7.48 (m, 5H), 7.28 (d, 1H), 4.61 (s, 2 H), 2.53-2.49 (m, 3H), 0.79-0.93 (m, 9H), −0.06-−0.04 (m, 6H).

### Description 53

### (6-Methyl-3-phenyl-2-pyridinyl)methanol (D53)

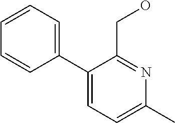

A solution of 2-({[(1,1-dimethylethyl)(dimethyl)silyl]oxy}methyl)-6-methyl-3-phenylpyridine D52 (0.99 g) in TBAF (1.0 M solution in THF) (10 ml, 10.00 mmol) was stirred at room temperature for 30 minutes. The solvent was removed in vacuo and the residue was taken up in water (15 ml). The resulting solution was washed with DCM. The combined organic layers were dried (Na₂SO₄) and evaporated. The residual yellow oil was purified by flash chromatography on silica gel (Companion, 120 g cartridge with Cy/EtOAc from Cy 100 to Cy 70/30 elution) to afford the title compound D53 (0.53 g) as a white solid. HPLC (walk up): rt=2.31 min. C₁₃H₁₃NO 199. ¹H NMR (400 MHz, DMSO-d₆) δ ppm 7.60 (d, 1H), 7.34-7.51 (m, 5H), 7.27 (d, 1H), 5.12 (m, 1H), 4.33-4.45 (m, 2H), 2.54-2.49 (m, 3H).

### Description 54

### 6-Methyl-3-phenyl-2-pyridinecarboxylic acid (D54)

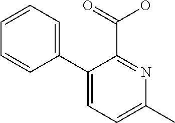

To a solution of (6-methyl-3-phenyl-2-pyridinyl)methanol D53 (0.2 g) in water (3 ml) was added dropwise a solution of KMnO₄ (0.206 g, 1.305 mmol) in water (7 ml) at 5-10° C. with vigorous stirring, then the reaction mixture was stirred at room temperature overnight and then filtered through a plugh of celite (MnO₂ was removed). The filtrate was concentrated under reduced pressure. The unreacted substance was removed by extraction with DCM. The pH of the aqueous layer was adjusted to pH=5.5 with 2 N HCl and the product was extracted with DCM. The organic layers were collected, dried over Na₂SO₄ and evaporated to afford the title compound D54 (0.056 g) as a white solid. MS: (ES/+) m/z: 214 (M+1). C₁₃H₁₁NO₂ requires 213. ¹H NMR (400 MHz, DMSO-d₆) ppm 13.23 (br. s., 1H), 7.78 (d, 1H), 7.50-7.35 (m, 6H), 2.53 (s, 3H).

### Description 55

### 2-methylfuro[3,4-b]pyridine-5,7-dione (D55)

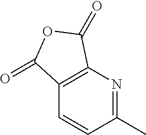

In a 100 ml round-bottomed flask 6-methyl-2,3-pyridinedicarboxylic acid (10 g, 55.2 mmol) and acetic anhydride (26 ml, 276 mmol) were added and heated at 100° C. under nitrogen for 5 hours. After this time the volatiles were removed under vacuum to give the title compound D55 (8.2 g) as a slightly brown solid. ¹H NMR (400 MHz, DMSO-d₆) δ ppm 8.41 (d, 1H), 7.82 (d, 1H), 2.73 (s, 3H).

### Description 56

### 6-methyl-2-[(methyloxy)carbonyl]-3-pyridinecarboxylic acid (D56)

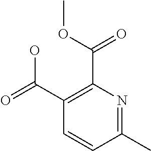

2-methylfuro[3,4-b]pyridine-5,7-dione D55 (3 g) was added portionwise over 5 minutes to stirred MeOH (20 ml) at 0° C. The mixture was stirred at 0° C. for 30 minutes then at room temperature for other 2.5 hours. The solution was evaporated at reduced pressure and the residue recrystallized from toluene (50 ml). The solid was filtered and dried under high vacuum for 30 minutes, obtaining a first batch of the title compound D56 (1.16 g) as pale brown solid. From the toluene solution new solid precipitated: this solid was filtered and dried under high vacuum for 30 minutes, obtaining a second batch of the title compound D56 (352 mg) as pale yellow solid. The toluene solution was then evaporated at reduced pressure and the residue recrystallized again from toluene (25 ml). The solid was filtered and dried under high vacuum for 30 minutes, obtaining a third batch of the title compound D56 (615 mg) as pale yellow solid. UPLC (Basic GEN_QC): rt=0.23 minutes, peak observed: 195 (M+1). C₉H₉NO₄ requires 196. ¹H NMR (400 MHz, DMSO-d₆) δ ppm 13.61 (br. s., 1H), 8.09-8.31 (m, 1H), 7.51 (m, 1H), 3.82 (s, 3H), 2.55 (s, 3H).

### Description 57

### methyl 3-({[(1,1-dimethylethyl)oxy]carbonyl}amino)-6-methyl-2-pyridinecarboxylate (D57)

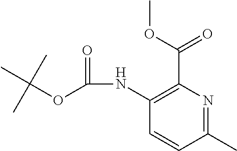

6-methyl-2-[(methyloxy)carbonyl]-3-pyridinecarboxylic acid D56 (1.15 g) was suspended in toluene (40 ml) and DIPEA (1.25 ml, 7.16 mmol) was added, causing the complete dissolution of the solid. This mixture was stirred 10 minutes at room temperature, then diphenyl azidophosphate (1.35 ml, 6.26 mmol) was added in one portion and the mixture was stirred at reflux for 1 hour. The solution was cooled at room temperature and t-BuOH (2.5 ml, 26 mmol) was added in one portion. The mixture was then stirred at 70° C. for 1 hour and then cooled at room temperature, Et₂O (50 ml) was added and the resulting solution washed with NaHCO₃ saturated solution (3×60 mls). The water phases were joined together and back-extracted with Et₂O (50 mls). The two organic solutions were joined together, dried over Na₂SO₄ and evaporated at reduced pressure, obtaining the crude target material as pale yellow oil. This material was purified by flash chromatography on silica gel (Biotage, EtOAc/Cy from 10/90 to 70/30; Snap-100 g column). The title compound D57 (1.315 g) was obtained as white solid. UPLC (Basic GEN_QC): rt=0.68 minutes, peak observed: 267 (M+1). C₁₃H₁₈N₂O₄ requires 266. ¹H NMR (400 MHz, CDCl₃) δ ppm 10.13 (bs., 1H), 8.77 (d, 1H), 7.34 (d, 1H), 4.03 (s, 3H), 2.59 (s, 3H), 1.53-1.56 (m, 9H).

### Description 58

### methyl 3-amino-6-methyl-2-pyridinecarboxylate (D58)

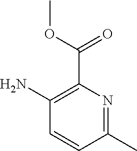

Methyl 3-({[(1,1-dimethylethyl)oxy]carbonyl}amino)-6-methyl-2-pyridinecarboxylate D57 (1.3 g) was dissolved in DCM (80 ml) and the mixture stirred at 0° C. A solution of TFA (5 ml, 64.9 mmol) in DCM (10 ml) was dropped into the cold mixture over 3 minutes. The resulting solution was left under stirring at 0° C. for 30 minutes, then the mixture was left still at room temperature overnight. TFA (4 ml, 51.9 mmol) dissolved in DCM (10 ml) was added over 3 minutes and the mixture stirred again at room temperature for 5 hours. The solution was loaded onto an SCX-25 g column and the column was eluted firstly with DCM (100 mls) and then MeOH (20 mls). The material was collected eluting with NH₃ (2M in MeOH, 100 mls) and after evaporation under reduced pressure of the ammonia solution it was obtained the title compound D58 (770 mg) was obtained as a white solid. UPLC (Basic GEN_QC): rt=0.44 minutes, peak observed: 167 (M+1). C₈H₁₀N₂O₂ requires 166. ¹H NMR (400 MHz, CDCl₃) δ ppm 7.14 (d, 1H), 7.01 (d, 1H), 3.99 (s, 3H), 2.52 (s, 3H).

### Description 59

### methyl 3-iodo-6-methyl-2-pyridinecarboxylate (D59)

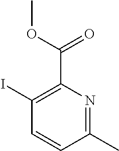

HCl 6 M solution in water (4.5 ml, 27.0 mmol) was added to methyl 3-amino-6-methyl-2-pyridinecarboxylate D58 (768 mg) and the resulting pale yellow mixture was sequentially diluted with water (4×5 ml) and chilled at 0° C. (internal temperature).

A solution of sodium nitrite (480 mg, 6.96 mmol) in water (2 ml) was dropped into the mixture over 1 minute. After this addition the mixture was stirred at 0° C. for 30 minutes, then a solution of KI (1.69 g, 10.18 mmol) in water (2 ml) was added over 1 minute, causing the formation of a dark violet crust (moderate gas evolution). The mixture was left under stirring for 1 hour: during this period the temperature passed from 0° C. to +5° C. EtOAc (50 ml) was then added to the stirred mixture, causing the dissolution of the dark solid. Water (50 ml) and EtOAc (50 ml) were added and the whole mixture was poured into a separator funnel. After the separation of the two phases, the water phase was extracted with EtOAc. All the organic phases were joined together and washed with NaHCO₃ saturated solution; the acidic water phase was neutralized by the addition of the previously used NaHCO₃ saturated solution and the resulting mixture extracted with EtOAc (2×50 mls). All the organic phases were joined together, dried over Na₂SO₄ and evaporated at reduced pressure, obtaining the crude target material as dark brown/violet oil. This material was purified by silica gel chromatography (Biotage SP4 Snap-100 g column, EtOAc/Cy from 10/90 to 30/70). The title compound D59 was obtained as a pale brown solid (1.1 g). UPLC (Basic GEN_QC): rt=0.68 minutes, peak observed: 278 (M+1). C₈H₈INO₂ requires 277. ¹H NMR (400 MHz, CDCl₃) δ ppm 8.12 (d, 1H), 7.01 (d, 1H), 4.01 (s, 3H), 2.58 (s, 3H).

### Description 60

### methyl 6-methyl-3-(2-pyrimidinyl)-2-pyridinecarboxylate (D60)

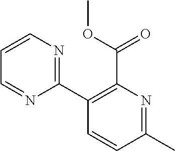

To a suspension of methyl 3-iodo-6-methyl-2-pyridinecarboxylate D59 (300 mg), CsF (329 mg, 2.166 mmol) and Pd(Ph₃P)₄ (50.0 mg, 0.043 mmol) in DMF (10 ml) stirred under nitrogen at room temperature was added 2-(tributylstannanyl)pyrimidine (480 mg, 1.299 mmol). The reaction mixture was stirred at 130° C. for 30 minutes at microwave Personal Chemistry. The reaction mixture was partitioned between EtOAc and aqueous NaHCO₃ saturated solution the combined organic phases were dried to give the crude product which was purified by silica gel chromatography (SNAP KP-NH 55 g; Cy/EtOAc 15 column volumes from 100/0 to 70/30). Collected fractions were evaporated to obtain the title compound D60 (101 mg) as white solid. UPLC (Basic GEN_QC): rt=0.56 minutes, peak observed: 230 (M+1). C₁₂H₁₁N₃O₂ requires 229. ¹H NMR (400 MHz, DMSO-d₆) δ ppm 8.92 (d, 2H), 8.49 (d, 1H), 7.44-7.63 (m, 2H), 3.75 (s, 3H), 2.57 (s, 3H).

### Description 61

### 6-methyl-3-(2-pyrimidinyl)-2-pyridinecarboxylic acid lithium salt (D61)

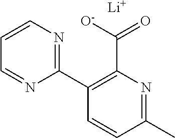

To a solution of methyl 6-methyl-3-(2-pyrimidinyl)-2-pyridinecarboxylate D60 (100 mg) in MeOH (4.5 ml) and water (1.1 ml) was added LiOH (13.58 mg, 0.567 mmol) and the resulting mixture was submitted to microwave irradiation at 60° C. for 85 minutes. After this time the solvents were removed under reduced pressure to give the title compound D61 (100 mg) as a white solid. C₁₁H₈N₃O₂.Li⁺ requires 221. ¹H NMR (400 MHz, DMSO-d₆) δ ppm 8.78 (m, 2H), 7.86 (m, 1H), 7.37 (m, 1H), 7.24 (m, 1H), 2.50 (s, 3H).

### Description 62

### 3-(5,5-Dimethyl-1,3,2-dioxaborinan-2-yl)-6-methyl-2-pyridinecarbonitrile (D62)

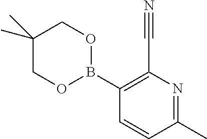

2,2,6,6-tetramethylpiperidine (3.49 ml, 20.52 mmol) was dissolved in dry THF (25 ml) under argon and stirred at −30° C.; BuLi (13.33 ml, 21.33 mmol) 1.6 M in hexane was added over 5 min (the temperature never exceeded −25° C.). The yellow solution was stirred at −30° C. for 20 min, then chilled at −78° C. and tris(1-methylethyl) borate (4.38 ml, 18.96 mmol) was added over 5 min (the temperature never exceeded −73° C.).

After 10 min at −78° C., 6-methyl-2-pyridinecarbonitrile (2.0 g, 16.93 mmol) dissolved in dry THF (14 ml) was added dropwise (over 20 min) maintaining internal temperature below −73° C. and the mixture became dark-brown. The mixture was stirred at −73° C. for 2 hours. The mixture was quenched with AcOH (2.374 ml, 41.5 mmol) dropwise at −73° C. (the temperature never exceeded −60° C. and the mixture became brilliant orange). The cooling bath was removed and the mixture left to reach the room temperature: during this period the mixture became thick and new THF (8 ml) had to be added in order to have a better stirring. The mixture was stirred 10 min at room temperature then 2,2-dimethyl-1,3-propanediol (2.409 g, 23.13 mmol) was added in one portion and the mixture stirred at room temperature overnight. The solvent was evaporated and the orange residue taken-up with DCM (100 ml) and 10% water solution of KH₂PO₄ (100 ml). The phases were separated and the water phase was back-extracted with DCM (50 ml). The combined organic phases were washed with 10% water solution of KH₂PO₄ (50 ml). The DCM was evaporated. The residue was dissolved in Et₂O (100 ml) and extracted with NaOH 0.05 M (5×50 ml, boronic ester in water phase). The aqueous phases were joined together and the pH was adjusted between pH=4 and pH=5 with 10% water solution of KH₂PO₄ (50 ml). The so obtained yellow solution was extracted with EtOAc (3×200 mls). All the organics joined together were dried (Na₂SO₄) and evaporated the title compound D62 (2.29 g) of as yellow oil, that solidified on standing. C₁₂H₁₅BN₂O₂ requires 230. ¹H NMR (400 MHz, CDCl₃) δ ppm 7.97-8.15 (m, 1H), 7.31-7.36 (m, 1H), 3.85 (m, 4H), 2.52-2.73 (s, 3H), 0.97-1.10 (m, 6 H).

### Description 63

### 6-Methyl-3-(2-pyrimidinyl)-2-pyridinecarbonitrile (D63)

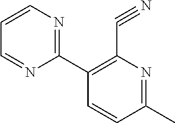

A) Isopropylmagnesium chloride-LiCl (37.9 ml, 36.5 mmol) was added portion wise (in overall 10 min) to a solution of 3-bromo-6-methyl-2-pyridinecarbonitrile (4 g, 20.30 mmol) in THF (150 ml) cooled to −70° C. (internal temperature). The reaction was kept to that temperature for 15 min. Then it was allowed to gently warm up to −40° C. in overall 1 hour. Then, it was cooled to −78° C. and zinc chloride (3.32 g, 24.36 mmol) was added. The resulting mixture was allowed to warm up to room temperature in 1 hour. Pd(Ph₃P)₄ (2.346 g, 2.030 mmol), 2-chloropyrimidine (3 g, 26.2 mmol) were added and the mixture was refluxed (external temperature 100° C.) until complete consumption of starting chloropyrimidine (3 hours). The reaction mixture was cooled to room temperature and poured into water (200 ml) cooled to 10° C. It was then extracted with EtOAc (5×200 mls). The collected organic phases, containing large amount of colloid material and water, were washed with brine (200 ml). The water phase was filtered over a gouch, and the solid material was washed with further EtOAc (2×300 mls). The collected organic phases were dried overnight over Na₂SO₄, filtered and concentrated to give (7 g) the crude material which was purified (Biotage Sp1 over a 240 g Silica Anolgix column, with a 25 g pre-column) to give the title compound D63 as yellow solid (1.8 g). UPLC (Acid GEN_QC_SS): rt=0.58 minutes, peak observed: 197 (M+1). C₁₁H₈N₄ requires 196.

B) An alternative method to make D63 is: 3-(5,5-dimethyl-1,3,2-dioxaborinan-2-yl)-6-methyl-2-pyridinecarbonitrile D62 (50.6 mg) was dissolved 1,4-Dioxane (1 ml) under nitrogen in a vial, then 2-bromopyrimidine (42.0 mg, 0.264 mmol), CsF (67 mg, 0.441 mmol), Pd(Ph₃P)₄ (12 mg, 10.38 μmol) and CuI (7 mg, 0.037 mmol) were added in sequence. The vial was then capped and stirred at 65° C., after 1 hour the solvent was removed at reduced pressure and the residue partitioned between AcOEt (10 ml) and NaHCO₃ (saturated solution, 10 ml). The phases were separated and the water was extracted with AcOEt (2×10 mls). The organic fraction were joined together, dried over Na₂SO₄ and evaporated at reduced pressure, obtaining an orange oily residue which was purified (Biotage, Snap 25 g silica gel column, AcOEt/Cy from pure Cy to 50:50 in 10 column volumes) to obtain the title compound D63 as pale yellow solid (27.6 mg).

### Description 64

### 6-Methyl-3-(2-pyrimidinyl)-2-pyridinecarboxylic acid (D64)

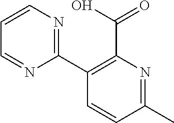

A) 6-methyl-3-(2-pyrimidinyl)-2-pyridinecarbonitrile D63 (0.8 g) was reacted in 6 M aqueous HCl (40 ml, 240 mmol) at 80° C. for 3 hours, then solvent was removed under vacuum, and the resulting crude was purified (70 g Varian C18 column conditioning with MeOH (120 mls), then water (120 mls), loading in water, washing with water (200 mls), product eluted with 100% MeOH) to give the title compound D64 (0.6 g) as yellow solid. UPLC (Acid GEN_QC_SS): rt=0.30 minutes, peak observed: 216 (M+1). C₁₁H₉N₃O₂ requires 217. ¹H NMR (400 MHz, DMSO-d₆) δ ppm 13.07 (bs, 1H), 8.78-9.01 (m, 2H), 8.39 (m, 1H), 7.39-7.67 (m, 2H), 2.56-2.67 (s, 3H).

B) An alternative method to make D64 is as follows: 6-methyl-3-(2-pyrimidinyl)-2-pyridinecarbonitrile D63 (0.481 g) was suspended in EtOH (5 ml) and a solution of NaOH (0.490 g, 12.26 mmol) in water (5 ml) was added. The yellow mixture was stirred at 100° C. overnight. The yellow solution was cooled to 25° C. and HCl 6 M (1.0 ml) was added dropwise till pH=4.5. The solvent was removed to give a yellow powder that was dried at 50° C./vacuum for 1.5 hours to give the title compound D64 (1.242 g).

### Description 65

### methyl 6-methyl-3-(4-methyl-1,3-thiazol-2-yl)-2-pyridinecarboxylate (D65)

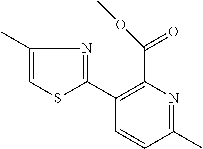

4-methyl-2-(tributylstannanyl)-1,3-thiazole (150 mg, 0.386 mmol) was dissolved in 1,4-Dioxane (2.5 ml). To the stirred solution of methyl 3-iodo-6-methyl-2-pyridinecarboxylate D59 (100 mg) was added, followed by Pd(Ph₃P)₄ (41.7 mg, 0.036 mmol).

The resulting orange solution was heated into a microwave reactor at 120° C. for 30 minutes. The mixture was loaded onto an SCX-5 g column the column was eluted and after evaporation under reduced pressure of the solvent it was obtained the crude target material as colorless oil, which was then purified by flash chromatography on silica gel (Biotage SNAP-10 g silica gel column, EtOAc/Cy 25:75). It was obtained the title compound D65 as white solid (74 mg). UPLC (Acid GEN_QC): rt=0.62 minutes, peak observed: 249 (M+1). C₁₂H₁₂N₂O₂S requires 248. ¹H NMR (400 MHz, CDCl₃) δ ppm 7.97 (d, 1H), 7.33 (d, 1 H), 6.98 (s, 1H), 3.94 (s, 3H), 2.66 (s, 3H), 2.50 (s, 3H).

### Description 66

### 6-methyl-3-(4-methyl-1,3-thiazol-2-yl)-2-pyridinecarboxylate lithium salt (D66)

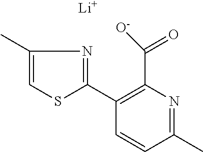

methyl 6-methyl-3-(4-methyl-1,3-thiazol-2-yl)-2-pyridinecarboxylate D65 (73 mg) was dissolved in EtOH (1 ml) into a capped vial, then a solution of LiOH (8.5 mg, 0.355 mmol) in water (0.5 ml) was added in one portion. The mixture was then stirred at room temperature for 3 hours. The solvent was evaporated at reduced pressure, obtaining the title compound D66 as pale yellow solid (73 mg). UPLC (Basic GEN_QC): rt=0.36 minutes, peak observed: 232 (M−1). C₁₁H₉N₂O₂S Li⁺ requires 233. ¹H NMR (400 MHz, DMSO-d₆) δ ppm 8.04 (d, 1H), 7.22 (d, 1H), 7.08 (d, 1H), 2.39 (s, 3H), 2.42 (s, 3H).

### Description 67

### methyl 2-chloro-6-methyl-3-pyridinecarboxylate (D67)

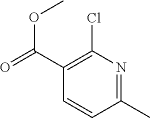

To a solution of 2-chloro-6-methyl-3-pyridinecarboxylic acid (8 g, 46.6 mmol) (available from Sigma-Aldrich #357847) in DCM (100 ml) and MeOH (50.0 ml) stirred under nitrogen at room temperature was added TMS-diazomethane 2 M in hexane (46.6 ml, 93 mmol). The reaction mixture was stirred at room temperature for 20 minutes. The solvents were removed to give the title compound D67 (7 g). MS: (ES/+) m/z: 186 (M+1). C₈H₈ClNO₂ requires 185. ¹H NMR (400 MHz, CDCl₃) δ ppm 8.10 (d, 1H), 7.18 (d, 1H), 3.96 (s, 3H), 2.61 (s, 3H).

### Description 68

### methyl 2-ethenyl-6-methyl-3-pyridinecarboxylate (D68)

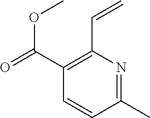

To a solution of methyl 2-chloro-6-methyl-3-pyridinecarboxylate D67 (2 g), Pd(Ph₃P)₄ (0.436 g, 0.377 mmol) in 1,4-Dioxane (15 ml) stirred under nitrogen at room temperature was added tributyl(ethenyl)stannane (3.76 g, 11.85 mmol) neat in one charge. The reaction mixture was stirred at microwave Personal Chemistry at 100° C. for 30 minutes. The solvent was removed to give the crude product which was purified by flash chromatography on silica (Companion: 120 g column, gradient elution from Cy to Cy/EtOAc 1:1) to afford the title compound D68 (1.9 g). UPLC (Basic GEN_QC): rt=0.73 minutes. peak observed: 178 (M+1). C₁₀H₁₁NO₂ requires 177. ¹H NMR (400 MHz, CDCl₃) δ ppm 8.08 (d, 1H), 7.66 (m, 1H), 7.12 (d, 1H), 6.52 (m, 1H), 5.59 (m, 1H), 3.93 (s, 3H), 2.63 (s, 3H).

### Description 69

### 2-ethenyl-6-methyl-3-(3-methyl-1,2,4-oxadiazol-5-yl)pyridine (D69)

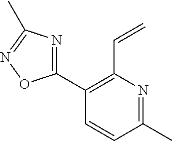

To a suspension of NaH 60% oil dispersion (0.903 g, 22.57 mmol) and molecular sieves 4 Å in dry THF (10 ml) stirred under nitrogen at room temperature acetamide oxime (0.836 g, 11.29 mmol) was added and the reaction stirred at room temperature for 30 minutes then a solution of methyl 2-ethenyl-6-methyl-3-pyridinecarboxylate D68 (1 g) in dry THF 10 ml was added in one charge. The reaction mixture was heated at the microwave Personal Chemistry at 100° C. for 30 minutes. NaHCO₃ saturated aqueous solution was added and the aqueous extracted with EtOAc, the organic passed through a hydrophobic frit, the solvent removed to give the crude product which was purified by flash chromatography on silica (80 g column, gradient elution from Cy to Cy/EtOAc 40/60) to afford the title compound D69 (308 mg). UPLC (Basic GEN_QC): rt=0.78 minutes. Peak observed: 202 (M+1). C₁₁H₁₁N₃O requires 201. ¹H NMR (400 MHz, CDCl₃) δ ppm 8.21 (d, 1H), 7.83 (m, 1H), 7.22 (d, 1H), 6.65 (m, 1H), 5.69 (m, 1H), 2.67 (s, 3H), 2.52 (s, 3H).

### Description 70

### 6-methyl-3-(3-methyl-1,2,4-oxadiazol-5-yl)-2-pyridinecarbaldehyde (D70)

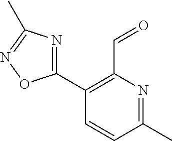

To a solution of 2-ethenyl-6-methyl-3-(3-methyl-1,2,4-oxadiazol-5-yl)pyridine D69 (100 mg) in THF (3 ml) and water (4.5 ml) stirred under nitrogen at room temperature was added a solution of OsO₄ 4% in water (0.39 ml, 0.05 mmol) and after 5 minutes in one charge sodium periodate (319 mg, 1.491 mmol). The reaction mixture was stirred at room temperature for 2 hours. The mixture was poured into a separatory funnel washed with brine and the aqueous extracted with EtOAc, the phases were separated on a hydrophobic frit, the combined organic solvent was removed to give the crude product which was purified by flash chromatography on silica gel (25 g column, gradient elution from Cy to Cy/EtOAc 80/20) to afford the title compound D70 (93 mg). UPLC (Basic GEN_QC): rt 1=0.50 minutes, rt 2=0.55 minutes, peaks observed: 204 (M+1). C₁₀H₉N₃O₂ requires 203. ¹H NMR (400 MHz, CDCl₃) δ ppm 10.55 (s, 1H), 8.21 (m, 1H), 7.53 (m, 1H), 2.78 (s, 3H), 2.52-2.56 (m, 3H).

### Description 71

### 6-methyl-3-(3-methyl-1,2,4-oxadiazol-5-yl)-2-pyridinecarboxylic acid (D71A/D71B)

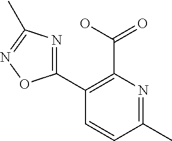

A) To a solution of 6-methyl-3-(3-methyl-1,2,4-oxadiazol-5-yl)-2-pyridinecarbaldehyde D70 (90 mg) in THF (3.00 ml) and water (6 ml) stirred at 0° C. was added solid NaOH (17.72 mg, 0.443 mmol) and after 10 minutes KMnO₄ (140 mg, 0.886 mmol) in one charge. The reaction mixture was stirred for 10 minutes. While still cold the reaction mixture was filtered on celite and the celite washed with HCl 1 M water solution and water. The aqueous filtrate at pH 1 was passed through a 50 g C18 column (MeOH, water to condition, water and then MeOH to elute) to afford the title compound D71A (70 mg). MS: (ES/−) m/z: 218 (M−1). C₁₀H₉N₃O₃ requires 219. ¹H NMR (400 MHz, CDCl₃) δ ppm 8.02 (d, 1H), 7.60 (d, 1H), 2.77 (s, 3H), 2.55 (s, 3H).

B) An alternative method to make D71 is: 6-methyl-3-(3-methyl-1,2,4-oxadiazol-5-yl)-2-pyridinecarbaldehyde D70 (0.89 mg) was dissolved in a mixture of DMSO (10 ml) and pH=3 buffer solution (3 ml) and the solution was cooled to 0° C. A 1 M solution of NaClO₂ in water (16 ml) was added; the solution turned to pale yellow and after the addition was left stirring at room temperature for 2 hours. New pH=3 buffer solution (1.5 ml) was added and the stirring was continued for 1 hour. The mixture was eluted through a 70 g C18 cartridge (preconditioned with MeOH and then with water; eluted with water and then with MeOH). The methanol fractions were joined and evaporated under reduced pressure to afford the title compound D71B (0.89 g).

### Description 72

### 2-chloro-N-(2-hydroxybutyl)-6-methyl-3-pyridinecarboxamide (D72)

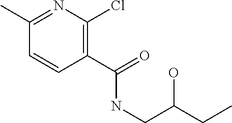

2-chloro-6-methyl-3-pyridinecarboxylic acid (2.5 g, 14.57 mmol) (available from Sigma-Aldrich #357847) was dissolved in DMF (35 ml) and DIPEA (7.63 ml, 43.7 mmol) was added. To this mixture TBTU (5.15 g, 16.03 mmol) was added in one portion and the resulting orange solution was stirred 45 minutes at room temperature. 1-amino-2-butanol (2.5 g, 28.0 mmol) was then added dissolved in DMF (5 ml) and the resulting mixture stirred at room temperature for 90 minutes. The mixture was then stored into the fridge over the weekend. The mixture was partitioned between NaHCO₃ saturated solution and Et₂O; the water layer was extracted with Et₂O. The water layer was then extracted with EtOAc. The organic phases deriving from the Et₂O extractions were joined and dried over Na₂SO₄ and evaporated at reduced pressure; the oily residue was dried under high vacuum at 45° C. for 2 hours, obtaining a first batch of crude material purified by flash chromatography on silica gel (Biotage 100 g column, EtOAc/Cy from 30:70 to 75:25). The organic phases deriving from the EtOAc extractions were joined and dried over Na₂SO₄ and evaporated at reduced pressure; the oily residue was dried under high vacuum at 45° C. for 1 hour, obtaining a second batch of crude material, purified by flash chromatography on silica gel (Biotage 340 g column, EtOAc/Cy from 30:70 to 75:25). The fractions eluted performing the two purifications were joined together and then evaporated at reduced pressure it was obtained the title compound D72 as pale yellow oil (3.62 g). UPLC (Basic GEN_QC): rt=0.45 minutes, peaks observed: 243 (M+1). C₁₁H₁₅ClN₂O₂ requires 242. ¹H NMR (400 MHz, DMSO-d₆) δ ppm 8.45 (m, 1H), 7.77 (m, 1H), 7.33 (m, 1H), 4.69 (m, 1H), 3.43-3.61 (m, 1H), 3.05-3.30 (m, 2H), 2.48 (s, 3H), 1.51 (m, 1H), 1.18-1.42 (m, 1H), 0.90 (t, 3H).

### Description 73

### 2-chloro-6-methyl-N-(2-oxobutyl)-3-pyridinecarboxamide (D73)

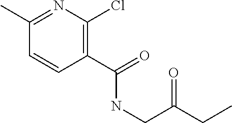

2-chloro-N-(2-hydroxybutyl)-6-methyl-3-pyridinecarboxamide D72 (3.62 g) was dissolved in DCM (100 ml), then, to the stirred solution, Dess-Martin periodinane (6.75 g, 15.91 mmol) was added portionwise over 5 minutes. The mixture was stirred at room temperature for 45 minutes (white suspension). The mixture was then partitioned between NaHCO₃ saturated solution and DCM; water layer extracted with DCM. The organic phases were joined, dried over Na₂SO₄ and evaporated at reduced pressure, obtaining the crude target material as pale yellow solid (7.2 g). This material was stored in the fridge overnight and was purified by flash chromatography on silica gel (Snap-340 g column, EtOAc/Cy from 20:80 to 80:20) to give the title compound D73 (3.11 g) as white solid. UPLC (Basic GEN_QC): rt=0.50 minutes. peaks observed: 241 (M+1). C₁₁H₁₃ClN₂O₂ requires 240. ¹H NMR (400 MHz, DMSO-d₆) δ ppm 8.82 (m, 1H), 7.81 (m, 1H), 7.37 (m, 1H), 4.09 (d, 2H), 3.30-3.35 (s, 3H), 2.53-2.59 (m, 2H), 0.97 (t, 3H).

### Description 74

### 2-chloro-3-(5-ethyl-1,3-oxazol-2-yl)-6-methylpyridine (D74)

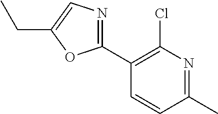

2-Chloro-6-methyl-N-(2-oxobutyl)-3-pyridinecarboxamide D73 (3.051 g) was dissolved in THF (100 ml) and Burgess reagent (3.104 g, 13.03 mmol) was added in one portion. The pale yellow solution was stirred at room temperature for 4.5 hours, then new Burgess reagent (0.41 g, 1.72 mmol) was added and the mixture stirred at 60° C. for 1.5 hours, the solvent was evaporated at reduced pressure and the residue partitioned between NaHCO₃ saturated solution and EtOAc; water layer was extracted with EtOAc. The organic phases were joined and dried over Na₂SO₄ and evaporated at reduced pressure, obtaining the crude target material, which was then purified by flash chromatography on silica gel (Snap-100 g column, EtOAc/Cy from 20:80 to 90:10). After evaporation at reduced pressure it was obtained the title compound D74 (1.7 g) colourless oil, which slowly solidified upon standing at room temperature and the unreacted starting material. UPLC (Basic GEN_QC): rt=0.77 minutes. peaks observed: 223 (M+1). C₁₁H₁₁ClN₂O requires 222. ¹H NMR (400 MHz, CDCl₃) δ ppm 8.21 (d, 1H), 7.21 (d, 1H), 6.96 (s, 1H), 2.80 (m, 2H), 2.62 (s, 3 H), 1.35 (t, 3H).

### Description 75

### 2-ethenyl-3-(5-ethyl-1,3-oxazol-2-yl)-6-methylpyridine (D75)

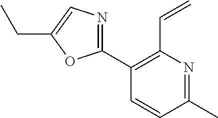

2-chloro-3-(5-ethyl-1,3-oxazol-2-yl)-6-methylpyridine D74 (168 mg), Pd(Ph₃P)₄ (70 mg, 0.061 mmol), 2-ethenyl-4,4,5,5-tetramethyl-1,3,2-dioxaborolane (0.2 ml, 1.179 mmol) and K₂CO₃ (209 mg, 1.509 mmol) were mixed together, then 1,4-dioxane (8 ml) and water (3 ml) were added. The mixture was stirred at 80° C. for 30 minutes. The mixture was stirred again at 80° C. for other 50 minutes. The solvents were evaporated at reduced pressure and the residue partitioned between NaHCO₃ saturated solution and Et₂O; water layer extracted with Et₂O. The organic phases were joined and dried over Na₂SO₄ and evaporated at reduced pressure, obtaining the crude target material which was purified by flash chromatography on silica gel (Snap-25 g column, EtOAc/Cy from 5:95 to 30:70). It was obtained the title compound D75 as white solid (135 mg). UPLC (Basic GEN_QC): rt=0.88 minutes, peaks observed: 215 (M+1). C₁₃H₁₄N₂O requires 214. ¹H NMR (400 MHz, CDCl₃) δ ppm 8.10 (m, 1H), 7.87 (m, 1H), 7.15 (m, 1H), 6.92 (s, 1H), 6.56 (m, 1H), 5.61 (m, 1H), 2.68-2.87 (m, 2H), 2.63 (s, 3H), 1.34 (t, 3H).

### Description 76

### 3-(5-ethyl-1,3-oxazol-2-yl)-6-methyl-2-pyridinecarbaldehyde (D76)

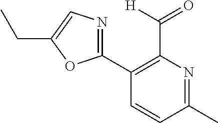

2-Ethenyl-3-(5-ethyl-1,3-oxazol-2-yl)-6-methylpyridine D75 (132 mg) was dissolved in THF (3 ml) and water (3 ml). To this stirred mixture a solution of OsO₄ 4% in water (0.390 ml, 0.050 mmol) was added over 30 seconds and the resulting mixture was then stirred at room temperature for 5 minutes. Sodium periodate (329 mg, 1.538 mmol) was then added in one portion and the resulting mixture was left to stir at room temperature for 70 minutes. The mixture was then partitioned between NaHCO₃ saturated solution and Et₂O; water layer extracted with Et₂O. The organic phases were joined and dried over Na₂SO₄ and evaporated at reduced pressure, obtaining the title compound D76 as brown solid (136 mg). UPLC (Basic GEN_QC): rt=0.65 minutes, peaks observed: 217 (M+1). C₁₂H₁₂N₂O₂ requires 216. ¹H NMR (400 MHz, CDCl₃) δ ppm 10.75 (s, 1H), 8.25 (d, 1H), 7.45 (d, 1H), 6.98 (s, 1 H), 2.76-2.91 (m, 2H), 2.74 (s, 3H), 1.35 (t, 3H).

### Description 77

### 3-(5-ethyl-1,3-oxazol-2-yl)-6-methyl-2-pyridinecarboxylic acid (D77)

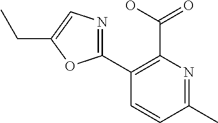

3-(5-ethyl-1,3-oxazol-2-yl)-6-methyl-2-pyridinecarbaldehyde D76 (550 mg) was dissolved in DMSO (5 ml) and citric pH=3 buffer solution (1.5 ml) and the mixture was chilled at 0° C. NaClO₂ 1 M in water (7 ml, 7.00 mmol) was dropped into the mixture over 10 minutes, then the stirring was continued at room temperature. New citric pH=3 buffer solution (1.5 ml), followed by new NaClO₂ 1 M in water (3 ml, 3.00 mmol) were dropped into the mixture, which was then stirred at room temperature for other 30 minutes, then the whole mixture has been stored in the fridge overnight. NaClO₂ 1 M in water (1 ml, 3.00 mmol) was dropped into the mixture, which was then stirred at room temperature for other 30 minutes. The whole dark mixture has been loaded onto a C18-70 g column (eluted with water then with MeOH). After evaporation at reduced pressure of the methanol fractions it was obtained the crude dark brown oil, which solidified by Et₂O (2 ml) addition. To this solid acetone (2.5 ml) and Et₂O (3 ml) were added. The solid was filtered and dried under high vacuum for 30 minutes, giving the dark brown solid (23 mg). To the solution Et₂O (8 ml) was added and the so obtained mixture was stored for 70 minutes into the fridge. This solid was filtered and washed with Et₂O (3 ml). All the organic solution (mother organic solution and Et₂O of washing) were joined, evaporated at reduced pressure and dried under high vacuum at 45° C. for 30 minutes, giving the title compound D77 as brown gum (362 mg). UPLC (Basic GEN_QC): rt=0.35 minutes, peaks observed: 231 (M−1). C₁₂H₁₂N₂O₃ requires 232. ¹H NMR (400 MHz, DMSO-d₆) δ ppm 8.20 (d, 1H), 7.50 (d, 1H), 7.05 (s, 1H), 2.61-2.82 (m, 3H), 2.55 (s, 3H), 1.23 (m, 3H).

### Description 78

### methyl 6-methyl-3-[(trimethylsilyl)ethynyl]-2-pyridinecarboxylate (D78)

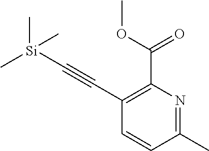

In a 10 ml round bottom flask methyl 3-iodo-6-methyl-2-pyridinecarboxylate D59 (200 mg), bis(triphenylphosphine)palladium(II) chloride (86 mg, 0.123 mmol), CuI (23.37 mg, 0.123 mmol) and DIPEA (0.391 ml, 2.238 mmol) were dissolved in DMF (2 ml) and then degassed. To this solution trimethylsilylacetylene (0.111 ml, 0.794 mmol) was added dropwise. After 30 min stirring at 23° C. water (2 ml) was added and extracted with EtOAc, the collected organic layer was dried (Na₂SO₄), filtered and evaporated under reduced pressure giving a brown oil which was purified by column chromatography on silica gel (SNAP KP-Sil 10 g; eluted with Cy/EtOAc 15 CV from 1/0 to 8/2) to give the title compound D78 (178 mg) as brown oil. UPLC (Basic GEN_QC): rt=0.92 minutes. peaks observed: 248 (M+1). C₁₃H₁₇NO₂Si requires 247. ¹H NMR (400 MHz, DMSO-d₆) δ ppm 7.92 (d, 1H), 7.46 (d, 1H), 3.88 (s, 3H), 0.10-0.34 (m, 9H).

### Description 79

### methyl 3-ethynyl-6-methyl-2-pyridinecarboxylate (D79)

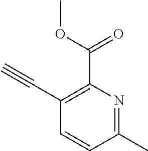

In a 25 ml round bottom flask methyl 6-methyl-3-[(trimethylsilyl)ethynyl]-2-pyridinecarboxylate D78 (178 mg) was dissolved in THF (4.8 ml) and treated with TBAF (1 M in THF) (0.935 ml, 0.935 mmol) at 0° C. The mixture was stirred for 15 minutes, then NaHCO₃ aqueous saturated solution (6 ml) and EtOAc (10 ml) were added. After the separation, the organic phase was washed with NaHCO₃ aqueous saturated solution. The collected aqueous layers were backextracted with EtOAc and the organic layers were joined together with the first EtOAc, dried (Na₂SO₄), filtered and evaporated under reduced pressure. The black oil obtained was purified by silica gel chromatography (SNAP KP-Sil 10 g cartridge; eluted with Cy/EtOAc 15 CV from 1/0 to 8/2). Collected and evaporated fractions gave the title compound D79 (83 mg) as solid.

UPLC (Basic GEN_QC): rt=0.57 minutes. peaks observed: 176 (M+1). C₁₀H₉NO₂ requires 175. ¹H NMR (400 MHz, DMSO-d₆) δ ppm 7.96 (d, 1H), 7.49 (d, 1H), 4.55 (s, 1H), 3.32 (s, 3H), 2.55 (s, 3H).

### Description 80

### methyl 6-methyl-3-(3-methyl-5-isoxazolyl)-2-pyridinecarboxylate (D80)

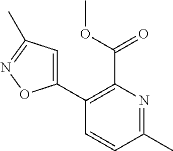

A solution of (1Z)—N-hydroxyethanimidoyl chloride (77 mg, 0.822 mmol) in toluene (2.2 ml) was cooled to 0° C. and methyl 3-ethynyl-6-methyl-2-pyridinecarboxylate D79 (60 mg) was added followed by TEA (0.119 ml, 0.856 mmol). The resulting mixture was stirred for 1 hour at 130° C. EtOAc (10 ml) and NH₄Cl aqueous saturated solution (5 ml) were added and after the separation the aqueous phase was extracted with EtOAc. Collected organic layers were dried (Na₂SO₄), filtered and evaporated under reduced pressure to give a brown solid which was purified by silica gel chromatography (SNAP KP-Sil 25 g; eluted with Cy/EtOAc from 1:0 to 6:4). Collected fractions gave the title compound D80 (74 mg) as white solid. UPLC (Basic GEN_QC): rt=0.62 minutes. peaks observed: 233 (M+1). C₁₀H₉NO₂ requires 232. ¹H NMR (500 MHz, DMSO-d₆) δ ppm 8.16 (d, 1H), 7.60 (s, 1 H), 6.74 (s, 1H), 3.85 (s, 3H), 2.56 (s, 3H), 2.29 (s, 3H).

### Description 81

### 6-methyl-3-(3-methyl-5-isoxazolyl)-2-pyridinecarboxylate lithium salt (D81)

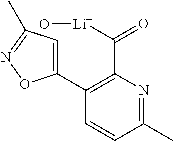

To a solution of methyl 6-methyl-3-(3-methyl-5-isoxazolyl)-2-pyridinecarboxylate D80 (74 mg) in EtOH (3.5 ml) and water (0.875 ml) was added LiOH (9.92 mg, 0.414 mmol) and the resulting mixture was stirred at 23° C. After 6.5 hours the solvents were removed under reduced pressure to give a white solid the title compound D81 (86 mg). UPLC (Basic GEN_QC): rt=0.33 minutes. peaks observed: 219 (M+1). C₁₁H₉N₂O₃—.Li⁺ requires 218. ¹H NMR (400 MHz, DMSO-d₆) δ ppm 7.90 (d, 1H), 7.12 (d, 1H), 6.80 (s, 1H), 2.44 (s, 3H), 2.26 (s, 3H).

### Description D82

### 2-chloro-N-(2-hydroxypropyl)-6-methyl-3-pyridinecarboxamide (D82)

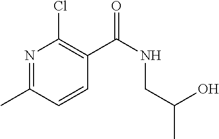

In a 100 ml round bottom flask 2-chloro-6-methyl-3-pyridinecarboxylic acid (1 g, 5.83 mmol) was added and dissolved in DMF (20 ml). To this solution DIPEA (5.09 ml, 29.1 mmol) and TBTU (2.246 g, 6.99 mmol) were added and the mixture stirred at room temperature for 30 minutes. After this time 1-amino-2-propanol (0.876 g, 11.66 mmol) was added and the resulting solution left under stirring at room temperature for 14 hour. After this time the reaction mixture was transferred into a separatory funnel containing brine and extracted with EtOAc. The combined organic phases were dried (Na₂SO₄) and evaporated to give the title compound D82 as crude yellow oil (2.1 g) that was used in the next step without further purification. MS: (ES/+) m/z: 229 (M+1). C₁₀H₁₃ClN₂O₂ requires 228.

### Description D83

### 2-chloro-6-methyl-N-(2-oxopropyl)-3-pyridinecarboxamide (D83)

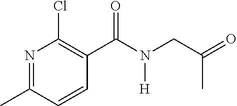

Into a 7 ml capped vial 2-chloro-N-(2-hydroxypropyl)-6-methyl-3-pyridinecarboxamide D82 (1.3 g), DCM (2 ml) and Dess-Martin periodinane (3.13 g, 7.39 mmol) were added and the resulting mixture left under stirring at room temperature for 4 hours. After this time solvent was removed and the crude purified by column chromatography on silica gel (DCM-MeOH=from 100/0 to 50/50). Collected fractions gave the crude title compound D83 (1.1 g) used without further purification. MS: (ES/+) m/z: 227 (M+1). C₁₀H₁₁ClN₂O₂ requires 226.

### Description D84

### 2-chloro-6-methyl-3-(5-methyl-1,3-oxazol-2-yl)pyridine (D84)

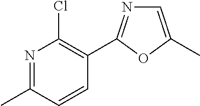

Into a 7 ml screw capped vial 2-chloro-6-methyl-N-(2-oxopropyl)-3-pyridinecarboxamide D83 (1.1 g) was dissolved in THF (2 ml) and Burgess reagent (1.041 g, 4.37 mmol) was added and the reaction mixture stirred at 50° C. for 2 hours. After this time volatiles were removed under vacuum and the crude purified by column chromatography on silica gel (flash master, silica NH₂ cartridge, Cy/EtOAc=from 100/0 to 80/20) to give the title compound D84 (430 mg) as an off-white solid. MS: (ES/+) m/z: 209 (M+1). C₁₀H₉ClN₂O requires 208.

### Description D85

### 2-ethenyl-6-methyl-3-(5-methyl-1,3-oxazol-2-yl)pyridine (D85)

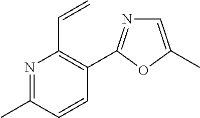

Into a microwave vial 2-chloro-6-methyl-3-(5-methyl-1,3-oxazol-2-yl)pyridine D84 (0.365 g), Pd(Ph₃P)₄ (0.091 g, 0.079 mmol) were added and dissolved in 1,4-dioxane (5 ml). The mixture was degassed and filled with nitrogen, then tributyl(vinyl)tin (0.506 ml, 1.732 mmol) was added and the reaction mixture was stirred at 95° C. for 1.5 hours. The mixture was filtered through a celite pad washed with EtOAc (20 ml), solvent was removed under vacuum to give the title compound D85 (1.15 g) as a dark yellow oil. This material was used in the next step without further purification. UPLC (Basic GEN_QC): rt=0.79 minutes, peak observed: 201 (M+1). C₁₂H₁₂N₂O requires 200.

### Description 86

### 6-methyl-3-(5-methyl-1,3-oxazol-2-yl)-2-pyridinecarbaldehyde (D86)

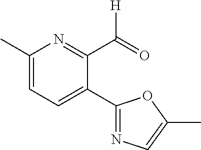

Into a 7 ml screw capped vial 2-ethenyl-6-methyl-3-(5-methyl-1,3-oxazol-2-yl)pyridine D85 (1.15 g), was dissolved in THF (10 ml) and water (15 ml) was added followed by osmium tetroxide 2.5% wt solution in methyl-2-propanol (3.61 ml, 0.287 mmol). After 5 minutes under stirring sodium periodate (1.843 g, 8.61 mmol) was added and the mixture left under stirring at room temperature. The mixture was transferred into a separatory funnel with EtOAc and brine and the mixture extracted with EtOAc. The combined organic phases were dried (Na₂SO₄) and evaporated under vacuum to give the title compound D86 (0.343 g) as brown crude oil. UPLC (Basic GEN_QC): rt=0.55 minutes, peak observed: 203 (M+1). C₁₁H₁₀N₂O₂ requires 202.

### Description D87

### 6-methyl-3-(5-methyl-1,3-oxazol-2-yl)-2-pyridinecarboxylic acid (D87A/D87B)

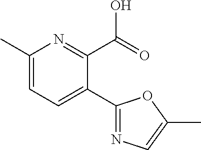

A) In a 250 ml flask 6-methyl-3-(5-methyl-1,3-oxazol-2-yl)-2-pyridinecarbaldehyde D86 (343 mg) was dissolved in THF (3.50 ml) and water (7 ml), to the mixture sodium hydroxide (67.8 mg, 1.696 mmol) and potassium permanganate (536 mg, 3.39 mmol) were added and stirred at room temperature for 5 min. The organic solvent was removed under vacuum and the residue was filtered on a celite pad, washed with aq 1M HCl. The aqueous layer was charged on Varian C18 column (50 g, washed with 5 CV of water and eluted with 1 CV of MeOH) to give a yellow oil, (126 mg). It was purified by chromatography on silica gel (KP-Sil 25 g column; DCM/MeOH/AcOH 94/4/2). To give a colorless vitreous solid which was triturated with Et₂O (1 ml) yielding, the title compound D87A (30 mg) as white solid. MS (ES−) peak observed 217 (M−1), C₁₁H₁₀N₂O₃ requires 218. HPLC walkup rt=4.40 minutes.

B) An alternative method to make D87 is: 6-methyl-3-(5-methyl-1,3-oxazol-2-yl)-2-pyridinecarbaldehyde D86 (92 mg, 0.455 mmol) was dissolved in DMSO (2 ml). The mixture was cooled at 0° C. and pH=3 buffer solution (3 ml) was added, then a solution of sodium chlorite (103 mg, 1.137 mmol) in water (2.5 ml) was added dropwise in 5 min. The reaction was allowed to reach room temperature and was stirred for 1 hour. Reaction was diluted with water (10 ml) and back-extracted with EtOAc (10 ml×10). Only a little amount of product was extracted in organic phases and the title compound still remained in the aqueous phase. The combined organic phases were dried over Na₂SO₄, and evaporated affording an orange oil. The aqueous phase was charged on a C18 cartridge (conditioned with MeOH and then with H₂O, eluted with water and MeOH) to afford a yellow oil which was jointed with the previous orange oil obtained from the organic phases. The dark oil was repurified using a C18 Cartridge (25 g conditioned with one of MeOH and then with of Water, eluted with water and MeOH) affording a dark oil which was redissolved in Et₂O. Evaporation of the solvent afforded a crude brown solid which was triturated with a mixture of Acetone (1 ml) and Et₂O (2 ml), the orange liquor was removed and the product was dried in vacuo to afford the title compound D87B (62 mg). MS: (ES+) m/z: 219 (M+1). C₁₁H₁₀N₂O₃ requires 218. ¹H NMR (400 MHz, DMSO-d₆) δ ppm 13.45 (br. s., 1H), 8.18 (m, 1H), 7.49 (m, 1H), 7.03 (m, 1H), 2.54 (s, 3H), 2.34-2.39 (m, 3H).

### EXAMPLES

### Example 1

### 2-[((2S)-1-{[3-(Ethyloxy)-6-methyl-2-pyridinyl]carbonyl}-2-piperidinyl)methyl]-6-fluoro-8-methylimidazo[1,2-a]pyridine hydrochloride (E1)

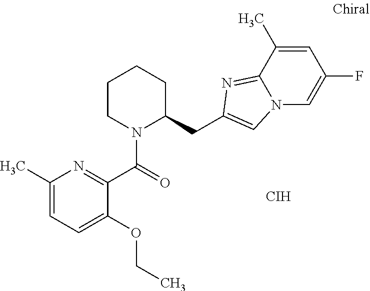

To a solution of 3-(ethyloxy)-6-methyl-2-pyridinecarboxylic acid D37 (0.0278 g) and TBTU (0.0519 g, 0.162 mmol, 2.000) in dry DMF (1.5 ml) was added under nitrogen at room temperature DIPEA (56 μl, 0.323 mmol, 4.0 equiv.) and the reaction mixture was stirred for 20 minutes. Then a solution of 6-fluoro-8-methyl-2-[(2S)-2-piperidinylmethyl]imidazo[1,2-c]pyridine D12 (0.020 g of the crude material obtained in the Description 12) in dry DMF (1.5 ml) was added under nitrogen and the reaction mixture was stirred overnight at room temperature. The reaction was stopped; the solvent was evaporated to dryness. DCM and a saturated aqueous solution of NH₄Cl were added. The aqueous layer was extracted 4 times with DCM. The organic layers were dried over Na₂SO₄ and evaporated. The crude compound was purified by Fraction Lynx (method with a basic aqueous phase for the gradient water/CH₃CN). The free base of the title compound E1 obtained as a yellow film (0.0278 g). MS: (ES/+) m/z: 411 (M+1). C₂₃H₂₇FN₄O₂ requires 410.

2-[((2S)-1-{[3-(Ethyloxy)-6-methyl-2-pyridinyl]carbonyl}-2-piperidinyl)methyl]-6-fluoro-8-methylimidazo[1,2-a]pyridine (0.0248 g) was dissolved in 2 ml of DCM. The resulting clear solution was cooled down to 0° C. HCl 1 M in Et₂O (0.108 mmol, 3 equiv) was then added dropwise to this solution. The reaction mixture was stirred at 0° C. for 10 minutes and at room temperature for 30 minutes. Purity of the reaction was checked by LC-MS (with hydrolysis of the chlorydrate salt in the mobile aqueous phase). The solvent was removed. The title compound E1 (0.0234 g) was obtained as a yellow powder. MS: (ES/+) m/z: 411 (M-HCl+1). C₂₃H₂₇FN₄O₂.HCl requires 446. ¹H NMR (400 MHz, DMSO-d₆) δ ppm 8.46-8.60 (m, 1H), 7.79 (s, 1H), 7.01-7.43 (m, 3H), 5.01-5.19 (m, 1H), 3.72-4.08 (m, 2 H), 2.84-3.27 (m, 4H), 2.15-2.43 (m, 6H), 1.26-1.93 (m, 6H), 1.07-1.28 (m, 3H).

### Example 2

### 6-Fluoro-8-methyl-2-{[(2S)-1-({6-methyl-3-[(2-methylpropyl)oxy]-2-pyridinyl}carbonyl)-2-piperidinyl]methyl}imidazo[1,2-a]pyridine (E2)

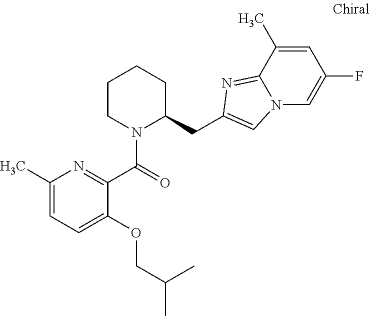

The solution of 6-methyl-3-[(2-methylpropyl)oxy]-2-pyridinecarboxylic acid D42 (0.030 g), 6-fluoro-8-methyl-2-[(2S)-2-piperidinylmethyl]imidazo[1,2-c]pyridine D12/13 (0.039 g), TBTU (0.0506 g, 0.158 mmol) and DIPEA (0.050 ml, 0.287 mmol) in anhydrous DMF (2 ml) was stirred at room temperature overnight. The reaction mixtures were evaporated to dryness, diluted with DCM (2 ml) and washed with saturated NaHCO₃ aqueous solution (2×3 ml). The organic layers were collected using a phase separator tube and concentrated. Purification by flash chromatography on silica gel (SP1, 25 M column, with DCM/MeOH) afforded the title compound E2 (0.0375 g) as a yellow solid. MS: (ES/+) m/z: 439 (M+1). C₂₅H₃₁FN₄O₂ requires 438. ¹H NMR (400 MHz, DMSO-d₆) δ ppm: 8.57-8.52 (m, 1H), 7.76-7.82 (s, 1H), 7.36-7.41 (d, 1H), 7.18-7.22 (d, 1H), 7.11-7.14 (m, 1H), 5.06-5.11 (m, 1H), 3.48-3.81 (m, 2H), 2.87-3.25 (m, 4H), 2.49-2.48 (s, 3H), 2.34-2.41 (s, 3H), 1.89-1.20 (m, 7H), 0.81-0.91 (d, 6H).

### Example 3

### 6,8-Dimethyl-2-{[(2S)-1-({6-methyl-3-[(2-methylpropyl)oxy]-2-pyridinyl}carbonyl)-2-piperidinyl]methyl}imidazo[1,2-a]pyridine (E3)

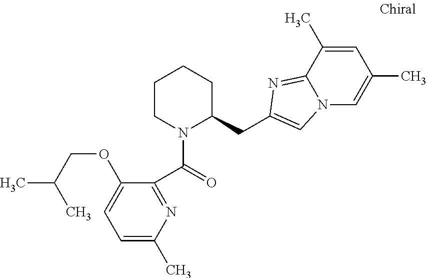

6-Methyl-3-[(2-methylpropyl)oxy]-2-pyridinecarboxylic acid D42 (0.0263 g) was dissolved in 1 ml of DMF, to the solution TBTU (0.0471 g, 0.147 mmol), DIPEA (0.110 ml, 0.629 mmol) were added and the solution was left stirring at room temperature for 30 minutes. Then 6,8-dimethyl-2-[(2S)-2-piperidinylmethyl]imidazo[1,2-a]pyridine D17 (0.0255 g) dissolved in 1 ml of DMF was added at 0° C. and the reaction was left stirring at room temperature for 2 hours. The reaction mixture was diluted with saturated NaHCO₃ aqueous solution and washed with DCM, the organic layers were washed with brine/ice and filtered through a phase separator, the solvent removed in vacuo. The crude was purified by flash chromatography (SP4, 25 M NH cartridge eluting with EtOAc 100%). The solvent was removed in vacuo obtaining the title compound E3 (0.042 g). MS: (ES/+) m/z: 435 (M+1). C₂₆H₃₄N₄O₂ requires 434. ¹H NMR (400 MHz, CDCl₃) δ ppm: 7.71-7.75 (m, 1H), 7.64 (s, 1H), 7.02-7.09 (m, 2H), 6.75-6.80 (m, 1H), 5.32-5.43 (m, 1H), 3.45-3.74 (m, 2H), 3.13-3.42 (m, 3H), 2.91-3.03 (m, 1H), 2.57 (s, 3H), 2.49 (s, 3H), 2.25 (s, 3H), 1.51-1.92 (m, 7H), 0.77-0.89 (m, 6H).

### Example 4

### 8-Methyl-2-[((2S)-1-{[6-methyl-3-(propyloxy)-2-pyridinyl]carbonyl}-2-piperidinyl)methyl]imidazo[1,2-a]pyridine (E4)

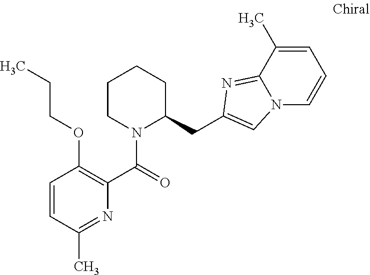

6-Methyl-3-(propyloxy)-2-pyridinecarboxylic acid D40 (0.0234 g) was dissolved in 1 ml of DMF then TBTU (0.049 g, 0.153 mmol) and DIPEA (0.114 ml, 0.654 mmol) were added and the reaction was stirred for 40 minutes. 8-Methyl-2-[(2S)-2-piperidinylmethyl]imidazo[1,2-a]pyridine D4 (0.025 g) was added in each reaction and the stirring was continued for 2 hours. DMF was removed under vacuum and the residue was taken up with 2 ml of DCM. This organic solution was washed with 1 ml of NaHCO₃ aqueous saturated solution, dried over Na₂SO₄ anhydrous, filtered and concentrated under vacuum to dryness. The resulting crude product was purified by flash chromatography (Biotage SP, NH column size 25+M, using EtOAc as eluent). It was recovered the title compound E4 (0.040 g). MS: (ES/+) m/z: 407 (M+1). C₂₄H₃₀N₄O₂ requires 406. ¹H NMR (400 MHz, DMSO-d₆) δ ppm: 8.20-8.52 (m, 1H), 7.77-7.90 (m, 1H), 6.54-7.45 (m, 4 H), 5.06-5.21 (m, 1H), 3.66-3.96 (m, 2H), 2.89-3.26 (m, 4H), 2.38 (s, 3H), 2.08-2.27 (m, 3H), 1.13-1.88 (m, 8H), 0.80-0.95 (m, 3H).

### Example 5

### 2-{[(2S)-1-({3-[(Cyclopropylmethyl)oxy]-6-methyl-2-pyridinyl}carbonyl)-2-piperidinyl]methyl}-8-methylimidazo[1,2-a]pyridine (E5)

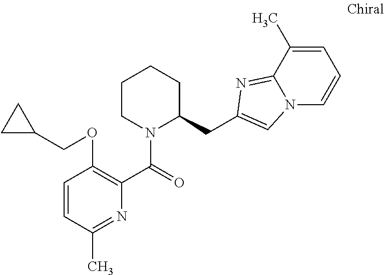

Following a similar procedure to that described for Example 4, 3-[(cyclopropylmethyl)oxy]-6-methyl-2-pyridinecarboxylic acid D41 (0.0248 g) and 8-methyl-2-[(2S)-2-piperidinylmethyl]imidazo[1,2-a]pyridine D4 (0.025 g) were reacted to afford the title compound E5 (0.035 g). MS: (ES/+) m/z: 419 (M+1). C₂₅H₃₀N₄O₂ requires 418. ¹H NMR (400 MHz, DMSO-d₆) δ ppm: 8.33 (d, 1H), 7.80 (s, 1H), 7.40 (d, 1H), 7.21 (d, 1H), 6.99 (d, 1H), 6.75 (t, 1H), 5.09-5.18 (m, 1H), 3.74-3.87 (m, 2H), 3.18-3.29 (m, 2H), 2.88-3.11 (m, 2H), 2.48 (s, 3H), 2.32 (s, 3H), 1.31-1.90 (m, 6H), 1.04-1.15 (m, 1H), 0.44-0.54 (m, 2H), 0.21-0.32 (m, 2H).

### Example 6

### 8-methyl-2-{[(2S)-1-({6-methyl-3-[(1-methylethyl)oxy]-2-pyridinyl}carbonyl)-2-piperidinyl]methyl}imidazo[1,2-a]pyridine (E6)

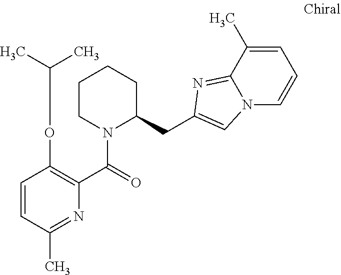

Following a similar procedure to that described for example 5, 6-methyl-3-[(1-methylethyl)oxy]-2-pyridinecarboxylic acid D39 (0.0234 g) and 8-methyl-2-[(2S)-2-piperidinylmethyl]imidazo[1,2-a]pyridine D4 (0.025 g) were reacted to afford the title compound E6 (0.041 g). MS: (ES/+) m/z: 407 (M+1). C₂₄H₃₀N₄O₂ requires 406. ¹H NMR (400 MHz, DMSO-d₆) δ ppm 8.30-8.36 (m, 1H), 7.80 (s, 1H), 7.36-7.46 (m, 1H), 7.21 (d, 1H), 6.97-7.03 (m, 1H), 6.72-6.80 (m, 1H), 5.08-5.20 (m, 1H), 4.54-4.65 (m, 1H), 2.85-3.29 (m, 4H), 2.40 (s, 3H), 2.33 (s, 3H), 1.31-1.85 (m, 6H), 1.11-1.27 (m, 6H)

### Example 7

### 2-[((2S)-1-{[4-Chloro-3-(ethyloxy)-6-methyl-2-pyridinyl]carbonyl}-2-piperidinyl)methyl]-8-methylimidazo[1,2-a]pyridine hydrochloride (E7)

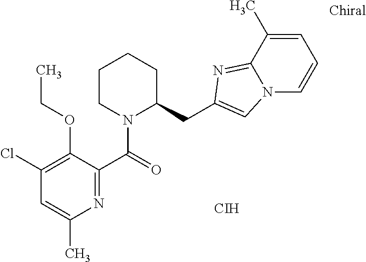

4-Chloro-3-(ethyloxy)-6-methyl-2-pyridinecarboxylic acid D43 (0.022 g of the crude material obtained in the Description 43) was dissolved in DMF (0.5 ml) and were added TBTU (0.0459 g, 0.143 mmol) then DIPEA (0.107 ml, 0.613 mmol). The resulting mixture was stirred 30 minutes at room temperature. To that solution was added a solution of 8-methyl-2-[(2S)-2-piperidinylmethyl]imidazo[1,2-a]pyridine D4 (0.0234 g) in DMF (0.5 ml) and stirred overnight. DCM (3 ml) and a saturated NaHCO₃ aqueous solution (2 ml) were added and the aqueous phase was extracted with DCM (2×2 ml). The organic layer was filtered through a phase separator cartridge and evaporated to obtain a yellow oil which was purified by flash chromatography (Biotage SP4, NH 12+M column, eluted with Cy/EtOAc from 100/0 to 40/60). 2-[((2S)-1-{[4-chloro-3-(ethyloxy)-6-methyl-2-pyridinyl]carbonyl}-2-piperidinyl)methyl]-8-methylimidazo[1,2-a]pyridine free base of the title compound E7 (0.018 g), was obtained like white solid. MS: (ES/+) m/z: 427 (M+1). C₂₃H₂₇ClN₄O₂ requires 426. ¹H NMR (400 MHz, DMSO-d₆) δ ppm 8.28-8.37 (m, 1H), 7.81 (s, 1H), 7.50 (s, 1H), 6.95-7.03 (m, 1H), 6.71-6.77 (m, 1H), 5.10-5.19 (m, 1H), 3.74-3.96 (m, 2H), 2.86-3.28 (m, 4H), 2.43 (s, 3H), 2.30 (s, 3H), 1.31-1.89 (m, 6H), 1.09-1.19 (m, 3H).

To a solution of 2-[((2S)-1-{[4-chloro-3-(ethyloxy)-6-methyl-2-pyridinyl]carbonyl}-2-piperidinyl)methyl]-8-methylimidazo[1,2-a]pyridine (0.015 g) in anhydrous DCM (1 ml) was added HCl 1 M in Et₂O (0.053 ml, 0.053 mmol) and stirred for 30 minutes. The solvent was removed under reduced pressure and then triturated with anhydrous Et₂O (1 ml), the solvent was removed by suction and the solid dried under reduced pressure. The title compound E7 (0.0155 g) was obtained like white solid. HPLC (walk-up): rt=4.18 min. MS: (ES/+) m/z: 427 (M-HCl+1). C₂₃H₂₇ClN₄O₂.HCl requires 463.

### Example 8

### 7,8-Dimethyl-2-{[(2S)-1-({6-methyl-3-[(2-methylpropyl)oxy]-2-pyridinyl}carbonyl)-2-piperidinyl]methyl}imidazo[1,2-a]pyridine (E8)

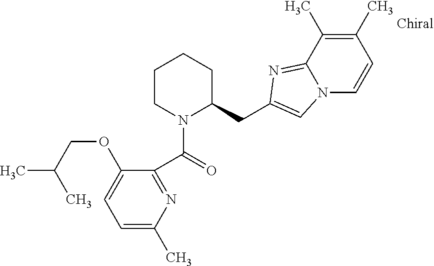

6-Methyl-3-[(2-methylpropyl)oxy]-2-pyridinecarboxylic acid D42 (0.034 g) was dissolved in 1 ml of DMF, to the solution TBTU (0.061 g, 0.190 mmol), DIPEA (0.142 ml, 0.814 mmol) were added and the solution was left stirring at room temperature for 30 minutes. Then 7,8-dimethyl-2-[(2S)-2-piperidinylmethyl]imidazo[1,2-a]pyridine D22 (0.033 g) dissolved in 1 ml of DMF was added at 0° C. and the reaction was left stirring at room temperature for 2 hours. The reaction mixture was diluted with saturated NaHCO₃ aqueous solution and washed with DCM, the organic layers were washed with brine/ice and filtered through a phase separator, the solvent removed in vacuo. The crude was purified by flash chromatography (SP4, NH 25M column, eluting with EtOAc 100%). The solvent was removed in vacuo obtaining the title compound E8 (0.0485 g). MS: (ES/+) m/z: 435 (M+1). C₂₆H₃₄N₄O₂ requires 434. ¹H NMR (400 MHz, CDCl₃) δ ppm 7.80-7.85 (m, 1 H), 7.64 (s, 1H), 6.95-7.17 (m, 2H), 6.48-6.56 (m, 1H), 5.33-5.42 (m, 1H), 3.54-3.75 (m, 2H), 2.93-3.42 (m, 4H), 2.53 (s, 3H), 2.49 (s, 3H), 2.31 (s, 3H), 1.50-1.91 (m, 7H), 0.77-0.90 (m, 6H).

### Example 9

### 2-{[(2S)-1-({3-[(Cyclopropylmethyl)oxy]-6-methyl-2-pyridinyl}carbonyl)-2-piperidinyl]methyl}-7,8-dimethylimidazo[1,2-a]pyridine hydrochloride (E9)

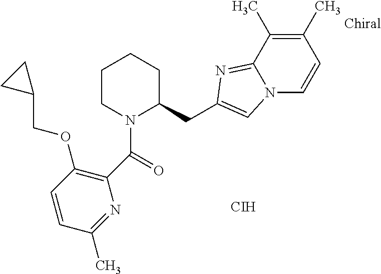

3-[(Cyclopropylmethyl)oxy]-6-methyl-2-pyridinecarboxylic acid D41 (0.0302 g) was dissolved in 1 ml of DMF, to the solution were added TBTU (0.0547 g, 0.170 mmol) and DIPEA (0.127 ml, 0.730 mmol) and solution was left stirring at room temperature for 1 hour. Then 7,8-dimethyl-2-[(2S)-2-piperidinylmethyl]imidazo[1,2-a]pyridine D22 (0.0296 g) dissolved in DMF (1 ml), was added at 0° C. and the reaction was left stirring at room temperature for 3 hours. The solvent was removed in vacuo and the crude was purified by flash chromatography (NH 25 M cartridge eluting from Cy 80%: EtOAc 20% for 2 CV, to EtOAc 100%). The fractions were collected, the solvent removed obtaining 2-{[(2S)-1-({3-[(cyclopropylmethyl)oxy]-6-methyl-2-pyridinyl}carbonyl)-2-piperidinyl]methyl}-7,8-dimethylimidazo[1,2-a]pyridine free base of the title compound E9 (0.044 g). HPLC (walk-up): rt=3.53 min. MS: (ES/+) m/z: 434 (M-HCl+1). C₂₆H₃₂N₄O₂ l requires 433. ¹H NMR (500 MHz, DMSO-d₆) δ ppm 8.20 (d, 1H), 7.70 (s, 1H), 7.11-7.41 (m, 2H), 6.67 (d, 1 H), 5.02-5.20 (m, 1H), 3.71-3.87 (m, 2H), 2.82-3.31 (m, 4H), 2.18-2.50 (m, 9H), 1.00-1.88 (m, 7H), 0.45-0.53 (m, 2H), 0.18-0.31 (m, 2H).

2-{[(2S)-1-({3-[(cyclopropylmethyl)oxy]-6-methyl-2-pyridinyl}carbonyl)-2-piperidinyl]methyl}-7,8-dimethylimidazo[1,2-a]pyridine (0.042 g) was dissolved in Et₂O (1 ml), to the solution was added dropwise HCl in Et₂O (1 ml, 1.000 mmol). The mixture was left stirring for 15 minutes, the solvent was removed and the residue washed several times with Et₂O. The solid was dried obtaining the title compound E9 (0.044 mg). HPLC (walk-up): rt=3.56 min.

MS: (ES/+) m/z: 434 (M-HCl+1). C₂₆H₃₂N₄O₂.HCl requires 469. ¹H NMR (400 MHz, DMSO-d₆) δ ppm 14.23 (br. s., 1H), 8.59-8.74 (m, 1H), 7.90-8.14 (m, 1H), 7.11-7.55 (m, 3H), 5.14-5.32 (m, 1H), 2.55-3.88 (m, 6H), 2.30-2.53 (m, 9H), 0.93-2.07 (m, 7H), 0.11-0.68 (m, 4H).

### Example 10

### 2-[((2S)-1-{[3-(Ethyloxy)-6-methyl-2-pyridinyl]carbonyl}-2-piperidinyl)methyl]-7,8-dimethylimidazo[1,2-a]pyridine hydrochloride (E10)

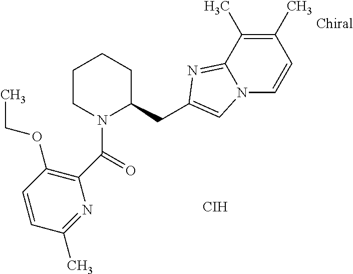

3-(Ethyloxy)-6-methyl-2-pyridinecarboxylic acid D37 (0.0264 g) was dissolved in 1 ml of DMF, to the solution were added TBTU (0.0547 g, 0.170 mmol) and DIPEA (0.127 ml, 0.730 mmol) and the solution was left stirring at room temperature for 1 hour. Then 7,8-dimethyl-2-[(2S)-2-piperidinylmethyl]imidazo[1,2-a]pyridine D22 (0.0296 g) dissolved in DMF (1 ml), was added at 0° C. and the reaction was left stirring at room temperature for 3 hours. The solvent was removed in vacuo and the residue purified by flash chromatography (NH 25 M cartridge eluting from Cy 80%: EtOAc 20% for 2 CV to EtOAc 100%). The fractions were collected, the solvent removed obtaining 2-[((2S)-1-{[3-(ethyloxy)-6-methyl-2-pyridinyl]carbonyl}-2-piperidinyl)methyl]-7,8-dimethylimidazo[1,2-a]pyridine the free base of the title compound E10 (0.0374 g). HPLC (walk-up): rt=3.24 min. MS: (ES/+) m/z: 408 (M+1). C₂₄H₃₀N₄O₂ requires 407. ¹H NMR (500 MHz, DMSO-d₆) δ ppm 8.20 (d, 1H), 7.68 (s, 1H), 7.12-7.43 (m, 2H), 6.65 (d, 1H), 5.06-5.14 (m, 1H), 3.93-4.06 (m, 2H), 2.83-3.25 (m, 4H), 2.39 (s, 3 H), 2.19-2.29 (m, 6H), 1.29-1.85 (m, 6H), 1.20-1.26 (m, 3H).

2-[((2S)-1-{[3-(ethyloxy)-6-methyl-2-pyridinyl]carbonyl}-2-piperidinyl)methyl]-7,8-dimethylimidazo[1,2-a]pyridine (0.0355 g) was dissolved in Et₂O (1 ml), HCl in Et₂O (1 ml, 1.000 mmol) was added dropwise and the mixture was left stirring at room temperature for 15 minutes. Then the solvent was removed and the residue was washed several times with Et₂O. The solid was dried obtaining the title compound E10 (0.0371 g). HPLC (walk-up): rt=3.22 min. MS: (ES/+) m/z: 408 (M-HCl+1). C₂₄H₃₀N₄O₂.HCl requires 442. ¹H NMR (400 MHz, DMSO-d₆) δ ppm 14.21 (br. s., 1H), 8.52-8.78 (m, 1H), 7.88-8.22 (m, 1H), 7.08-7.62 (m, 3H), 5.19-5.33 (m, 1H), 3.87-4.14 (m, 2H), 2.65-3.70 (m, 4H), 2.27-2.55 (m, 9H), 1.29-2.01 (m, 6H), 1.02-1.18 (m, 3H).

### Example 11

### 7,8-dimethyl-2-[((2S)-1-{[6-methyl-3-(propyloxy)-2-pyridinyl]carbonyl}-2-piperidinyl)methyl]imidazo[1,2-a]pyridine hydrochloride (E11a, E11b, E11c)

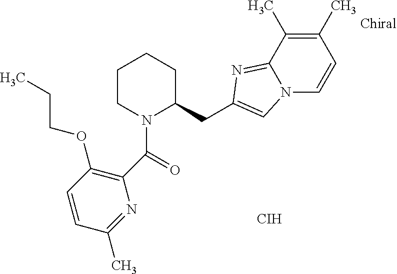

6-Methyl-3-(propyloxy)-2-pyridinecarboxylic acid D40 (0.307 g) was dissolved in 10 ml of DMF, to the solution TBTU (0.589 g, 1.835 mmol) and DIPEA (1.374 ml, 7.87 mmol) were added. The reaction was left stirring under N₂ atmosphere for 1 hour then 7,8-dimethyl-2-[(2S)-2-piperidinylmethyl]imidazo[1,2-a]pyridine D22 (0.319 g) was added and the reaction was left stirring 2 hours more. The solvent was removed in vacuo and the crude was purified by flash chromatography (SP4, 40M NH cartridges eluting from Cy 80%: EtOAc 20% to EtOAc 100%). The fractions were collected and the solvent removed in vacuo obtaining 7,8-dimethyl-2-[((2S)-1-{[6-methyl-3-(propyloxy)-2-pyridinyl]carbonyl}-2-piperidinyl)methyl]imidazo[1,2-a]pyridine free base of the title compound E11a (0.250 g). HPLC (walk-up): rt=4.5 min. C₂₅H₃₂N₄O₂ requires 420. ¹H NMR (400 MHz, DMSO-d₆) δ ppm 8.21 (d, 1H), 7.69 (s, 1H), 7.12-7.45 (m, 2H), 6.67 (d, 1H), 5.07-5.16 (m, 1 H), 3.71-3.99 (m, 2H), 2.81-3.27 (m, 4H), 2.20-2.46 (m, 9H), 1.21-1.89 (m, 8H), 0.83-0.95 (m, 3H).

Other fractions were collected separately obtaining after removing the solvent 7,8-dimethyl-2-[((2S)-1-{[6-methyl-3-(propyloxy)-2-pyridinyl]carbonyl}-2-piperidinyl)methyl]imidazo[1,2-a]pyridine free base of the title compound E11b (0.223 g). HPLC (walk-up): rt=4.44 min. C₂₅H₃₂N₄O₂ requires 420. ¹H NMR (400 MHz, DMSO-d₆) δ ppm 8.21 (d, 1H), 7.69 (s, 1H), 7.12-7.45 (m, 2H), 6.67 (d, 1H), 5.07-5.16 (m, 1H), 3.71-3.99 (m, 2H), 2.81-3.27 (m, 4H), 2.20-2.46 (m, 9H), 1.21-1.89 (m, 8H), 0.83-0.95 (m, 3H).

7,8-Dimethyl-2-[((2S)-1-{[6-methyl-3-(propyloxy)-2-pyridinyl]carbonyl}-2-piperidinyl)methyl]imidazo[1,2-a]pyridine (0.250 g, 0.594 mmol) was dissolved in Et₂O (5 ml), to the solution HCl in Et₂O (2 ml, 2.000 mmol) was added dropwise, the mixture was left stirring for 30 minutes then the solvent was removed in vacuo and the residue was treated with Et₂O. The solid was dried under vacuum obtaining the title compound E11a (0.299 g). HPLC (walk-up): rt=4.44 min. C₂₅H₃₂N₄O₂.HCl requires 457. ¹H NMR (400 MHz, DMSO-d₆) δ ppm 14.21 (br. s., 1H), 8.57-8.79 (m, 1H), 7.86-8.18 (m, 1H), 7.10-7.51 (m, 3H), 5.19-5.31 (m, 1H), 3.76-3.93 (m, 2H), 2.63-3.72 (m, 4H), 2.30-2.58 (m, 9H), 1.22-2.03 (m, 8H), 0.72-0.94 (m, 3H).

7,8-Dimethyl-2-[((2S)-1-{[6-methyl-3-(propyloxy)-2-pyridinyl]carbonyl}-2-piperidinyl)methyl]imidazo[1,2-a]pyridine (0.223 g, 0.530 mmol) was dissolved in Et₂O (5 ml) and DCM (1 ml), to the solution HCl in Et₂O (2 ml, 2.000 mmol) was added and the mixture was left stirring for 30 minutes. The solvent was removed in vacuo and the residue was washed several times with Et₂O. The solid was dried under vacuum at 40° C. overnight obtaining the title compound E11b (0.266 g). HPLC (walk-up): rt=4.40 min. C₂₅H₃₂N₄O₂.HCl requires 457. ¹H NMR (400 MHz, DMSO-d₆) δ ppm 14.21 (br. s., 1H), 8.57-8.79 (m, 1H), 7.86-8.18 (m, 1H), 7.10-7.51 (m, 3H), 5.19-5.31 (m, 1H), 3.76-3.93 (m, 2H), 2.63-3.72 (m, 4H), 2.30-2.58 (m, 9H), 1.22-2.03 (m, 8H), 0.72-0.94 (m, 3H).

7,8-Dimethyl-2-[((2S)-1-{[6-methyl-3-(propyloxy)-2-pyridinyl]carbonyl}-2-piperidinyl)methyl]imidazo[1,2-a]pyridine HCl salt E11b (0.266 g) was added to 7,8-dimethyl-2-[((2S)-1-{[6-methyl-3-(propyloxy)-2-pyridinyl]carbonyl}-2-piperidinyl)methyl]imidazo[1,2-a]pyridine HCl salt E11a (0.299 g) the solid was left under vacuum at 50° C. overnight to remove the residual traces of solvents obtaining the title compound E11c (0.540 g). HPLC (walk-up): rt=4.54 min. C₂₅H₃₂N₄O₂.HCl requires 457. ¹H NMR (400 MHz, DMSO-d₆) δ ppm 14.21 (br. s., 1H), 8.57-8.79 (m, 1H), 7.86-8.18 (m, 1H), 7.10-7.51 (m, 3H), 5.19-5.31 (m, 1H), 3.76-3.93 (m, 2H), 2.63-3.72 (m, 4H), 2.30-2.58 (m, 9H), 1.22-2.03 (m, 8H), 0.72-0.94 (m, 3H).

### Example 12

### 8-Fluoro-2-[((2S)-1-{[6-methyl-3-(propyloxy)-2-pyridinyl]carbonyl}-2-piperidinyl)methyl]imidazo[1,2-a]pyridine hydrochloride (E12)

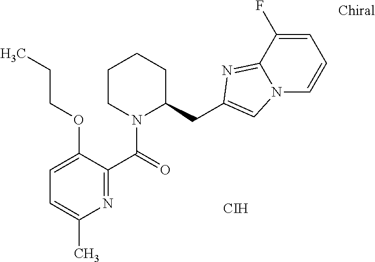

6-Methyl-3-(propyloxy)-2-pyridinecarboxylic acid D40 (0.0217 g) was dissolved in DMF (1 ml) and were added TBTU (0.358 g, 0.111 mmol) then DIPEA (0.117 ml, 0.670 mmol). The resulting mixture was stirred 1 hour at room temperature. To that solution was added a solution of 8-fluoro-2-[(2S)-2-piperidinylmethyl]imidazo[1,2-a]pyridine D9 (0.026 g of the crude material obtained in the Description 9) in DMF (1 ml) and stirred for 2.5 hours. DCM and a saturated NaHCO₃ aqueous saturated solution were added and the aqueous phase was extracted with DCM. The organic layer was filtered through a phase separator cartridge and evaporated to obtain a yellow oil which was purified by flash chromatography (Biotage SP4, NH, 12+M column, eluted with 35 CV of Cy/EtOAc from 1/0 to 2/8 and then with 15 CV of Cy/EtOAc 2/8). 8-fluoro-2-[((2S)-1-{[6-methyl-3-(propyloxy)-2-pyridinyl]carbonyl}-2-piperidinyl)methyl]imidazo[1,2-a]pyridine the free base of the title compound E12 (0.0332 g), was obtained as white solid. MS: (ES/+) m/z: 411 (M+1). C₂₃H₂₇FN₄O₂ requires 410. ¹H NMR (500 MHz, DMSO-d₆) δ ppm 8.36 (d, 1H), 7.92-7.98 (m, 1H), 7.40 (d, 1H), 7.21 (d, 1H), 7.06-7.12 (m, 1H), 6.74-6.85 (m, 1H), 5.07-5.18 (m, 1H), 3.74-3.96 (m, 2H), 2.88-3.28 (m, 4H), 2.39 (s, 3H), 1.29-1.85 (m, 8H), 0.83-0.92 (m, 3H).

To a solution of 8-fluoro-2-[((2S)-1-{[6-methyl-3-(propyloxy)-2-pyridinyl]carbonyl}-2-piperidinyl)methyl]imidazo[1,2-a]pyridine (0.031 g) in anhydrous DCM (1 ml) was added HCl 1 M in Et₂O (0.152 ml) The resulting mixture was stirred for 30 minutes and then the solvent was removed under reduced pressure to obtain a solid, which was triturated with Et₂O (1.000 ml). The solvent was removed by suction to afford the title compound E12 (0.0383 g) as white solid. HPLC (walk up): rt=3.26 min. MS: (ES/+) m/z: 411 (M-HCl+1). C₂₃H₂₇FN₄O₂.HCl requires 446.

### Example 13

### 8-Fluoro-2-{[(2S)-1-({6-methyl-3-[(2-methylpropyl)oxy]-2-pyridinyl}carbonyl)-2-piperidinyl]methyl}imidazo[1,2-a]pyridine hydrochloride (E13)

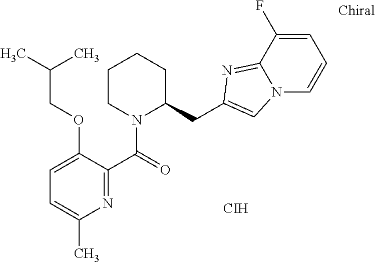

Following a similar procedure to that described for example 12, 6-methyl-3-[(2-methylpropyl)oxy]-2-pyridinecarboxylic acid D42 (0.023 g) and 8-fluoro-2-[(2S)-2-piperidinylmethyl]imidazo[1,2-a]pyridine D9 (0.026 g, of the crude material obtained in the Description 9) were reacted to afford 8-fluoro-2-{[(2S)-1-({6-methyl-3-[(2-methylpropyl)oxy]-2-pyridinyl}carbonyl)-2-piperidinyl]methyl}imidazo[1,2-c]pyridine the free base of the title compound E13 (0.0229 g). UPLC: rt=0.84 min. peak observed: 425 (M+1). C₂₄H₂₉FN₄O₂ requires 424. ¹H NMR (500 MHz, DMSO-d₆) δ ppm 8.33-8.38 (m, 1H), 7.93-7.97 (m, 1H), 7.40 (d, 1H), 7.20 (d, 1H), 6.99-7.11 (m, 1H), 6.74-6.86 (m, 1H), 5.09-5.18 (m, 1H), 3.66-3.79 (m, 2H), 2.89-3.28 (m, 4H), 2.39 (s, 3H), 1.28-1.92 (m, 7H), 0.84-0.93 (m, 6H).

Following a similar procedure to that described for example 12, starting from the free base 8-fluoro-2-{[(2S)-1-({6-methyl-3-[(2-methylpropyl)oxy]-2-pyridinyl}carbonyl)-2-piperidinyl]methyl}imidazo[1,2-c]pyridine (0.021 g) was obtained the title compound E13 (0.0258 g). HPLC (walk up): rt=3.58 min. MS: (ES/+) m/z: 425 (M-HCl+1). C₂₄H₂₉FN₄O₂.HCl requires 460.97.

### Example 14

### 2-{[(2S)-1-({3-[(Cyclopropylmethyl)oxy]-6-methyl-2-pyridinyl}carbonyl)-2-piperidinyl]methyl}-8-fluoroimidazo[1,2-a]pyridine hydrochloride (E14)

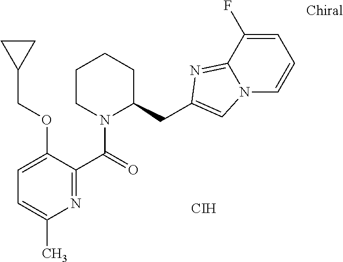

Following a similar procedure to that described for example 12, 3-[(cyclopropylmethyl)oxy]-6-methyl-2-pyridinecarboxylic acid D41 (0.023 g) and 8-fluoro-2-[(2S)-2-piperidinylmethyl]imidazo[1,2-a]pyridine D9 (0.026 g of the crude material obtained in the Description 9) were reacted to afford the free base of the title compound E14 (0.031 g). UPLC: rt=0.79 min. peak observed: 423 (M+1). C₂₄H₂₇FN₄O₂ requires 422. ¹H NMR (500 MHz, DMSO-d₆) δ ppm 8.36 (d, 1H), 7.93-7.97 (m, 1H), 7.38 (d, 1H), 7.19 (d, 1H), 6.98-7.11 (m, 1H), 6.74-6.84 (m, 1H), 5.09-5.17 (m, 1 H), 3.72-3.85 (m, 2H), 2.91-3.30 (m, 4H), 2.38 (s, 3H), 1.04-1.87 (m, 7H), 0.44-0.55 (m, 2H), 0.20-0.30 (m, 2H).

Following a similar procedure to that described for example 12, starting from the free base 2-{[(2S)-1-({3-[(cyclopropylmethyl)oxy]-6-methyl-2-pyridinyl}carbonyl)-2-piperidinyl]methyl}-8-fluoroimidazo[1,2-c]pyridine (0.029 g, 0.069 mmol) was obtained the title compound E14 (0.036.9 g). HPLC (walk up): rt=3.30 min. MS: (ES/+) m/z: 423 (M-HCl+1). C₂₄H₂₇FN₄O₂.HCl requires 459.

### Example 15

### 6,7-Dimethyl-2-{[(2S)-1-({6-methyl-3-[(2-methylpropyl)oxy]-2-pyridinyl}carbonyl)-2-piperidinyl]methyl}imidazo[1,2-a]pyridine hydrochloride (E15)

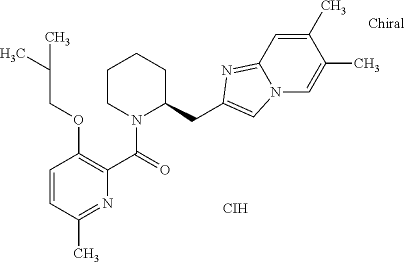

6-Methyl-3-[(2-methylpropyl)oxy]-2-pyridinecarboxylic acid D42 (0.0271 g) was dissolved in DMF (1 ml) and to the solution TBTU (0.0486 g, 0.151 mmol) and DIPEA (0.113 ml, 0.648 mmol) were added. The reaction was left stirring for 1 hour, then 6,7-dimethyl-2-[(2S)-2-piperidinylmethyl]imidazo[1,2-a]pyridine D27 (0.0263 g) in DMF (1 ml) was added. The solvent was removed in vacuo and the crude was purified by flash chromatography (SP4, 25 M NH cartridge eluting from Cy 80%: EtOAc 20% to EtOAc 100%). The fractions were collected, the solvent removed in vacuo obtaining 6,7-dimethyl-2-{[(2S)-1-({6-methyl-3-[(2-methylpropyl)oxy]-2-pyridinyl}carbonyl)-2-piperidinyl]methyl}imidazo[1,2-a]pyridine the free base of the title compound E15 (0.041 g) as a pale yellow foam. HPLC (walk up): rt=3.82 min. MS: (ES/+) m/z: 335 (M-HCl+1). C₂₆H₃₄N₄O₂ requires 334. ¹H NMR (500 MHz, DMSO-d₆) δ ppm 8.20-8.24 (m, 1H), 7.58-7.64 (m, 1H), 7.08-7.43 (m, 3H), 5.04-5.12 (m, 1H), 3.59-3.81 (m, 2H), 2.75-3.24 (m, 4H), 2.39 (s, 3H), 2.27 (s, 3H), 2.18 (s, 3H), 1.25-1.94 (m, 7H), 0.90 (d, 6 H).

6,7-Dimethyl-2-{[(2S)-1-({6-methyl-3-[(2-methylpropyl)oxy]-2-pyridinyl}carbonyl)-2-piperidinyl]methyl}imidazo[1,2-a]pyridine (0.039 g, 0.090 mmol) was dissolved in HCl in Et₂O (1 ml, 1.000 mmol), to the solution was added HCl in Et₂O (1 ml, 1.000 mmol) and the mixture was left stirring for 15 minutes. Then the solvent was removed in vacuo and the residue washed several times with Et₂O and dried in order to obtain the title compound E15 (0.043 g). HPLC (walk up): rt=3.77 min. C₂₆H₃₄N₄O₂.HCl requires 471. ¹H NMR (400 MHz, DMSO-d₆) δ ppm 14.28 (br. s., 1H), 8.71 (s, 1H), 8.03 (s, 1H), 7.74 (s, 1H), 7.12-7.49 (m, 2H), 5.14-5.24 (m, 1H), 2.71-3.90 (m, 6H), 2.24-2.59 (m, 9H), 1.24-2.07 (m, 7H), 0.71-0.94 (m, 6H).

### Example 16

### 3-Chloro-2-{[(2S)-1-({3-[(cyclopropylmethyl)oxy]-6-methyl-2-pyridinyl}carbonyl)-2-piperidinyl]methyl}-8-methylimidazo[1,2-a]pyridine hydrochloride (E16)

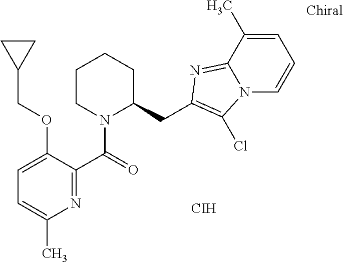

3-[(Cyclopropylmethyl)oxy]-6-methyl-2-pyridinecarboxylic acid D41 (0.017 g) was dissolved in DMF (1 ml) and were added TBTU (0.0375 g, 0.117 mmol) and then DIPEA (0.087 ml, 0.500 mmol). The resulting mixture was stirred 1 hour at room temperature. To that solution was added a solution of 3-chloro-8-methyl-2-[(2S)-2-piperidinylmethyl]imidazo[1,2-a]pyridine D29 (0.022 g, of the crude material obtained in the Description 29) in DMF (1 ml) and stirred for 2.5 hours. DCM and aqueous saturated solution of NaHCO₃ were added and the aqueous phase was extracted with DCM. The organic layer was filtered through a phase separator cartridge and evaporated to obtain a yellow oil which was purified by flash chromatography (via Biotage SP4, NH, 12+M column, eluted with Cy/EtOAc from 100/0 to 30/70). 3-chloro-2-{[(2S)-1-({3-[(cyclopropylmethyl)oxy]-6-methyl-2-pyridinyl}carbonyl)-2-piperidinyl]methyl}-8-methylimidazo[1,2-a]pyridine free base of the title compound E16 (0.012 mg), was obtained like brown solid. MS: (ES/+) m/z: 453 (M+1). C₂₅H₂₉ClN₄O₂ requires 452. ¹H NMR (500 MHz, DMSO-d₆) δ ppm 8.04-8.10 (m, 1H), 7.27-7.33 (m, 1H), 6.89-7.22 (m, 3H), 4.47-4.56 (m, 1H), 3.68-3.93 (m, 3H), 2.82-3.17 (m, 3H), 2.34 (s, 3H), 2.05-2.18 (m, 3H), 0.78-1.92 (m, 7H), 0.43-0.56 (m, 2H), 0.19-0.35 (m, 2H).

To a solution of 3-chloro-2-{[(2S)-1-({3-[(cyclopropylmethyl)oxy]-6-methyl-2-pyridinyl}carbonyl)-2-piperidinyl]methyl}-8-methylimidazo[1,2-a]pyridine (0.010 g, 0.022 mmol) in anhydrous DCM (1 ml) was added HCl (1M in Et₂O) (0.044 ml). The resulting mixture was stirred for 30 minutes and then the solvent was removed under reduced pressure to obtain a solid, which was triturated with Et₂O (1 ml). The solvent was removed by suction and the solid dried under vacuum at 40° C. to afford the title compound E16 (0.011 g) like white solid. HPLC (walk up): rt=3.76 min. MS: (ES/+) m/z: 453 (M-HCl+1). C₂₅H₂₉ClN₄O₂.HCl requires 489.

### Example 17

### 3-Chloro-2-[((2S)-1-{[3-(ethyloxy)-6-methyl-2-pyridinyl]carbonyl}-2-piperidinyl)methyl]-8-methylimidazo[1,2-a]pyridine hydrochloride (E17)

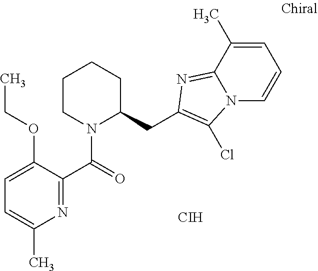

Following a similar procedure to that described for example 16, 3-(ethyloxy)-6-methyl-2-pyridinecarboxylic acid D37 (0.015 g) and 3-chloro-8-methyl-2-[(2S)-2-piperidinylmethyl]imidazo[1,2-a]pyridine D29 (0.022 g, of the crude material obtained in the Description 29) were reacted to afford 3-chloro-2-[((2S)-1-{[3-(ethyloxy)-6-methyl-2-pyridinyl]carbonyl}-2-piperidinyl)methyl]-8-methylimidazo[1,2-a]pyridine free base of the title compound E17 (0.012 g) like brown solid. UPLC: rt=0.81 min. peak observed: 427 (M+1). C₂₃H₂₇ClN₄O₂ requires 426. ¹H NMR (500 MHz, DMSO-d₆) δ ppm: 8.05-8.09 (m, 1H), 7.25-7.32 (m, 1H), 7.06-7.23 (m, 2H), 6.92 (t, 1H), 4.46-4.54 (m, 1 H), 3.75-4.08 (m, 3H), 2.80-3.28 (m, 3H), 2.34 (s, 3H), 2.05-2.18 (m, 3H), 1.32-1.90 (m, 5H), 1.13-1.29 (m, 3H), 0.87-1.08 (m, 1H).

Following a similar procedure to that described for example 16, starting from the free base 3-chloro-2-[((2S)-1-{[3-(ethyloxy)-6-methyl-2-pyridinyl]carbonyl}-2-piperidinyl)methyl]-8-methylimidazo[1,2-a]pyridine (0.010 g, 0.023 mmol) was obtained the title compound E17 (0.010 g) like white solid.

HPLC (walk up): rt=3.49 min. MS: (ES/+) m/z: 427 (M-HCl+1). C₂₃H₂₇ClN₄O₂.HCl requires 463.

### Example 18

### 2-{[(2S)-1-({3-[(Cyclopropylmethyl)oxy]-6-methyl-2-pyridinyl}carbonyl)-2-piperidinyl]methyl}-3,8-dimethylimidazo[1,2-a]pyridine hydrochloride (E18)

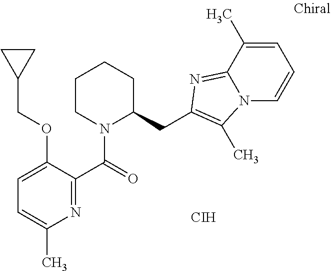

3-[(Cyclopropylmethyl)oxy]-6-methyl-2-pyridinecarboxylic acid D41 (0.021 g) was dissolved in DMF (0.5 ml) and were added TBTU (0.046 g, 0.144 mmol) then DIPEA (0.054 ml, 0.308 mmol). The resulting mixture was stirred 30 minutes at room temperature. To that solution was added a solution of 3,8-dimethyl-2-[(2S)-2-piperidinylmethyl]imidazo[1,2-a]pyridine D7 (0.025 g) in DMF (0.5 ml) and stirred overnight. DCM (3 ml) and a saturated NaHCO₃ aqueous solution (2 ml) were added and the aqueous phase was extracted with DCM (2×2 ml). The organic layer was filtered through a phase separator cartridge and evaporated to obtain an orange oil which was purified via Biotage SP4 (NH, 12+M column; eluted with 40 CV of Cy/EtOAc from 1/0 to 3/7). 2-{[(2S)-1-({3-[(cyclopropylmethyl)oxy]-6-methyl-2-pyridinyl}carbonyl)-2-piperidinyl]methyl}-3,8-dimethylimidazo[1,2-a]pyridine the free base of the title compound E18 (0.029 g) was obtained like white solid. MS: (ES/+) m/z: 433 (M+1). C₂₆H₃₂N₄O₂ requires 432. ¹H NMR (500 MHz, DMSO-d₆) δ ppm 7.95 (d, 1H), 7.28-7.35 (m, 1H), 7.10-7.23 (m, 1H), 6.89-7.05 (m, 1H), 6.70-6.76 (m, 1H), 4.45-4.56 (m, 1H), 3.65-3.96 (m, 3H), 2.74-3.24 (m, 3H), 2.48 (s, 3H), 2.32 (s, 3H), 2.12-2.24 (m, 3H), 0.89-1.93 (m, 7H), 0.41-0.57 (m, 2H), 0.18-0.36 (m, 2H).

To a solution of 2-{[(2S)-1-({3-[(cyclopropylmethyl)oxy]-6-methyl-2-pyridinyl}carbonyl)-2-piperidinyl]methyl}-3,8-dimethylimidazo[1,2-a]pyridine (27 mg) in anhydrous DCM (1 ml) was added HCl 1 M in Et₂O (0.124 ml, 0.124 mmol) and the resulting mixture was stirred for 30 minutes. The solvent was evaporated under reduced pressure and the white solid obtained was triturated with anhydrous MeOH (2 drops) and anhydrous Et₂O (1 ml), filtered by suction and dried under reduced pressure. The title compound E18 (0.029 g) was obtained like white solid. HPLC (walk up): rt=3.95 min. MS: (ES/+) m/z: 433 (M-HCl+1). C₂₆H₃₂N₄O₂.HCl requires 469.

### Example 19

### 2-[((2S)-1-{[6-Ethyl-3-(ethyloxy)-2-pyridinyl]carbonyl}-2-piperidinyl)methyl]-7,8-dimethylimidazo[1,2-a]pyridine hydrochloride (E19)

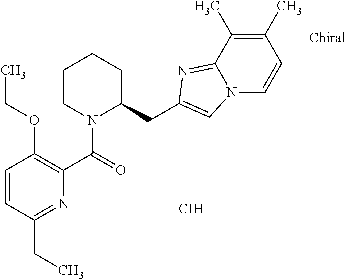

6-Ethyl-3-(ethyloxy)-2-pyridinecarboxylic acid D49 (0.024 g), TBTU (0.046 g, 0.144 mmol) and DIPEA (0.108 ml, 0.616 mmol) in DMF (1 ml) were left stirring at room temperature for 1 hour under N₂ atmosphere. Then to this solution 7,8-dimethyl-2-[(2S)-2-piperidinylmethyl]imidazo[1,2-a]pyridine D22 (0.025 g) dissolved in DMF (1 ml) was added dropwise and the reaction was left stirring at room temperature overnight. The solvent was removed in vacuo and the crude purified by flash chromatography (Sp4 25 M NH cartridge eluting from Cy100% to EtOAc 100%).

The fractions were collected obtaining 2-[((2S)-1-{[6-ethyl-3-(ethyloxy)-2-pyridinyl]carbonyl}-2-piperidinyl)methyl]-7,8-dimethylimidazo[1,2-a]pyridine the free base of the title compound E19 (0.039 g). HPLC (walk up): rt=3.53 min. MS: (ES/+) m/z: 421 (M+1). C₂₅H₃₂N₄O₂ requires 420. ¹H NMR (400 MHz, DMSO-d₆) δ ppm 8.15-8.27 (m, 1 H), 7.63 (s, 1H), 7.09-7.47 (m, 3H), 5.02-5.15 (m, 1H), 3.84-4.13 (m, 2H), 2.80-3.25 (m, 4H), 2.55-2.76 (m, 2H), 2.13-2.30 (m, 6H), 1.31-1.86 (m, 6H), 1.08-1.29 (m, 6H).

2-[((2S)-1-{[6-ethyl-3-(ethyloxy)-2-pyridinyl]carbonyl}-2-piperidinyl)methyl]-7,8-dimethylimidazo[1,2-a]pyridine (0.037 g) was dissolved in Et₂O (1 ml), then HCl in Et₂O (0.088 ml, 0.088 mmol) was added to the solution. The mixture was shaken for 15 minutes, the solvent was removed in vacuo and the residue washed several times with Et₂O, obtaining the title compound E19 (0.040 g).

HPLC (walk up): rt=3.56 min. MS: (ES/+) m/z: 421 (M-HCl+1). C₂₅H₃₂N₄O₂.HCl requires .¹H NMR (400 MHz, DMSO-d₆) d ppm 14.27 (br. s., 1H), 7.11-8.76 (m, 5H), 5.14-5.26 (m, 1H), 3.74-4.05 (m, 2H), 2.53-3.60 (m, 6H), 2.27-2.49 (m, 6H), 1.20-1.92 (m, 6 H), 1.02-1.17 (m, 6H).

### Example 20

### 6-fluoro-8-methyl-2-({(2S)-1-[(6-methyl-3-phenyl-2-pyridinyl)carbonyl]-2-piperidinyl}methyl)imidazo[1,2-a]pyridine (E20)

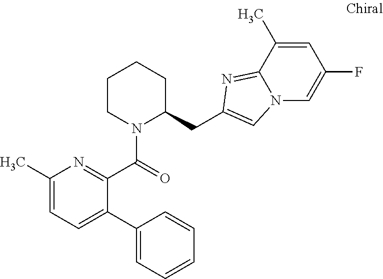

A solution of 6-methyl-3-phenyl-2-pyridinecarboxylic acid D54 (0.056 g), 6-fluoro-8-methyl-2-[(2S)-2-piperidinylmethyl]imidazo[1,2-a]pyridine D12/13 (0.071 g), TBTU (0.093 g, 0.289 mmol) and DIPEA (0.092 ml, 0.525 mmol) in anhydrous DMF (2 ml) was stirred at room temperature overnight. The reaction mixture was concentrated, diluted with DCM and washed with NaHCO₃ aqueous saturated solution. The organic layer was collected using a phase separator tube and concentrated. Purification by flash chromatography on silica gel (SP1, 25M column with DCM/MeOH, from DCM 100 to DCM/MeOH 95/5) afforded 6-fluoro-8-methyl-2-({(2S)-1-[(6-methyl-3-phenyl-2-pyridinyl)carbonyl]-2-piperidinyl}methyl)imidazo[1,2-a]pyridine E20 (0.044 g) as a pale yellow solid. MS: (ES/+) m/z: 443 (M+1). C₂₇H₂₇FN₄O requires 442. ¹H NMR (500 MHz, DMSO-d₆) δ ppm: 8.44-8.56 (m, 1H), 7.69-7.82 (m, 2H), 7.25-7.53 (m, 6H), 7.01-7.16 (m, 1H), 4.91-5.03 (m, 1H), 2.74-3.10 (m, 4H), 2.47-2.53 (m, 3H), 2.33 (s, 3H), 0.89-1.73 (m, 6H).

The following compounds were prepared using a similar procedure to that described for Example 20 (in some examples the solvent used was DCM instead of DMF and/or the order of addition of the reagents was different). Each compound was obtained by amide coupling of the corresponding [(2S)-2-piperidinylmethyl]imidazo[1,2-a]pyridine with the appropriate carboxylic acid. This is provided merely for assistance to the skilled chemist. The starting material may not necessarily have been prepared from the batch referred to. Unless specified the free base was not treated with the HCl solution to give the corresponding HCl salt.

| | Amide coupling | |
|---|---|---|
| No. | Reactants | Characterising data |
| E21 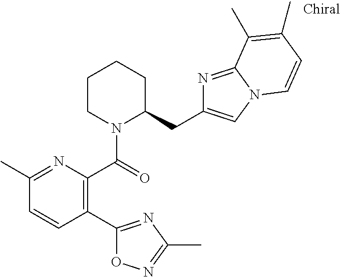 | D22 and D71 | 7,8-dimethyl-2-[((2S)-1-{[6-methyl-3-(3-methyl- 1,2,4-oxadiazol-5-yl)-2-pyridinyl]carbonyl}-2- piperidinyl)methyl]imidazo[1,2-a]pyridine HPLC-MS (Basic gradient): rt1 = 1.48 min, rt2 = 1.66 min. peaks observed: 445 (M + 1). C₂₅H₂₈N₆O₂ requires 444. ¹H NMR (400 MHz, DMSO-d₆) δ ppm 8.37 (d, 1 H), 8.21 (d, 1 H), 7.70 (s, 1 H), 7.47-7.59 (m, 1 H), 6.67 (d, 1 H), 4.47 (d, 1 H), 3.88-4.00 (m, 1 H), 2.85- 3.31 (m, 3 H), 2.60 (s, 3 H), 2.39 (s, 3 H), 2.21 (s, 3 H), 2.15 (s, 3 H), 1.36-1.91 (m, 6 H). |
| E22 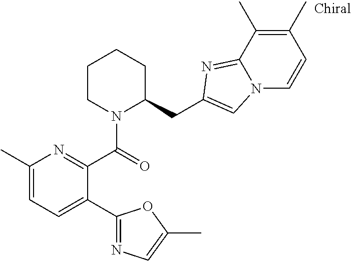 | D22 and D87 | 7,8-dimethyl-2-[((2S)-1-{[6-methyl-3-(5-methyl-1,3- oxazol-2-yl)-2-pyridinyl]carbonyl}-2- piperidinyl)methyl]imidazo[1 ,2-a]pyridine HPLC (walk up): rt = 3.66 min. peak observed: 444 (M + 1). C₂₆H₂₉N₅O₂ requires 443. ¹H NMR (500 MHz, DMSO-d₆) δ ppm 8.04-8.28 (m, 2 H), 7.72 (s, 1 H), 7.37-7.48 (m, 1 H), 7.01 (s, 1 H), 6.67 (d, 1 H), 5.01-5.14 (m, 1 H), 2.94-3.32 (m, 4 H), 2.42 (s, 3 H), 2.16-2.36 (m, 9 H), 1.19- 1.87 (m, 6 H). |
| E23 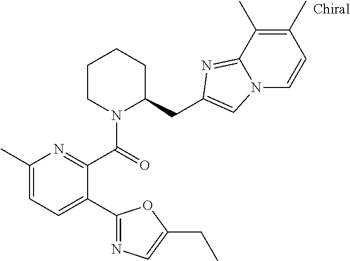 | D22 and D77 | 2-[((2S)-1-{[3-(5-ethyl-1,3-oxazol-2-yl)-6-methyl-2- pyridinyl]carbonyl}-2-piperidinyl)methyl]-7,8- dimethylimidazo[1,2-a]pyridine UPLC (Basic GEN_QC): rt1 = 0.76 min, rt2 = 0.84 min. peaks observed: 458 (M + 1). C₂₇H₃₁N₅O₂ requires 457. ¹H NMR (500 MHz, DMSO-d₆) δ ppm 8.04-8.29 (m, 2 H), 7.73 (s, 1 H), 7.29-7.48 (m, 1 H), 7.03 (s, 1 H), 6.51-6.77 (m, 1 H), 5.00-5.15 (m, 1 H), 2.94- 3.28 (m, 4 H), 2.57-2.78 (m, 2 H), 2.43 (s, 3 H), 2.17-2.31 (m, 6 H), 1.25-1.88 (m, 6 H), 1.12-1.25 (m, 3 H). |
| E24 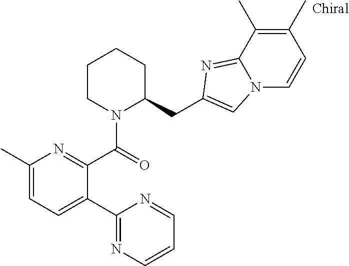 | D22 and D61 | 7,8-dimethyl-2-[((2S)-1-{[6-methyl-3-(2- pyrimidinyl)-2-pyridinyl]carbonyl}-2- piperidinyl)methyl]imidazo[1,2-a]pyridine UPLC (Basic GEN_QC): rt1 = 0.67 min, rt2 = 0.75 min. peaks observed: 441 (M + 1). C₂₆H₂₈N₆O requires 440. ¹H NMR (500 MHz, DMSO-d₆) δ ppm 8.79-8.88 (m, 2 H). 8.46 (d, 1 H), 8.20 (d, 1 H), 7.70 (s, 1 H), 7.31-7.49 (m, 2 H), 6.66 (d, 1 H), 4.91-5.07 (m, 1 H), 2.90-3.31 (m, 4 H), 2.56 (s, 3 H), 2.17-2.43 (m, 6H), 1.33-1.87 (m, 6 H) |
| E25 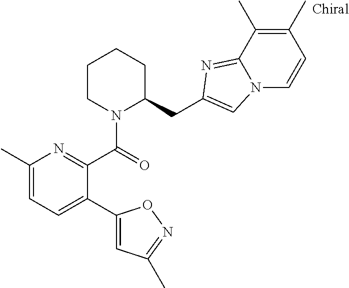 | D22 and D81 | 7,8-dimethyl-2-[((2S)-1-{[6-methyl-3-(3-methyl-5- isoxazolyl)-2-pyridinyl]carbonyl}-2- piperidinyl)methyl]imidazo[1,2-a]pyridine MS: (ES/+) m/z: 444 (M + 1). C₂₆H₂₉N₅O₂ requires 443. ¹H NMR (500 MHz, DMSO-d₆) δ ppm 8.05-8.13 (m, 2 H), 7.42-7.45 (m, 1 H), 7.40 (s, 1 H), 6.53- 6.58 (m, 1 H), 6.11 (br. s., 1 H), 4.43-4.53 (m, 1 H), 3.80-3.95 (m, 1 H), 2.80-3.25 (m, 3 H), 2.06-2.57 (m, 12 H), 1.20-1.87 (m, 6 H) |
| E26 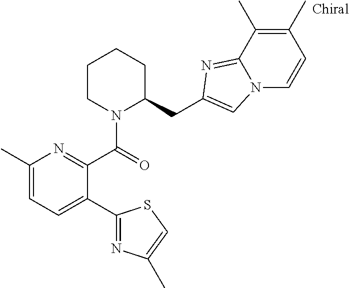 | D22 and D66 | 7,8-dimethyl-2-[((2S)-1-{[6-methyl-3-(4-methyl-1,3- thiazol-2-yl)-2-pyridinyl]carbonyl}-2- piperidinyl)methyl]imidazo[1,2-a]pyridine UPLC (Basic GEN_QC): rt1 = 0.75 min, rt2 = 0.83 min. peaks observed: 459 (M + 1). C₂₆H₂₉N₅OS requires 460. ¹H NMR (400 MHz, DMSO-d₆) δ ppm 8.05-8.26 (m, 2 H), 7.69 (s, 1 H), 7.19-7.47 (m, 2 H), 6.67 (d, 1 H), 4.99-5.12 (m, 1 H), 2.92-3.31 (m, 4 H), 2.12- 2.59 (m, 12 H), 1.18-1.87 (m, 6 H) |
| E27 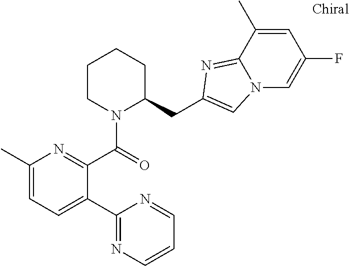 | D12/13 and D64 | 6-fluoro-8-methyl-2-[((2S)-1-{[6-methyl-3-(2- pyrimidinyl)-2-pyridinyl]carbonyl}-2- piperidinyl)methyl]imidazo[1,2-a]pyridine UPLC (Basic GEN_QC): rt1 = 0.67 min, rt2 = 0.74 min. peaks observed: 445 (M + 1). C₂₅H₂₅FN₆O requires 444. ¹H NMR (500 MHz, CDCl₃) δ ppm 8.67-8.71 (d, 2 H), 8.53-8.58 (d, 1 H), 7.79-7.98 (m, 2H), 7.27-7.30 (m, 1 H), 7.12-7.18 (t, 1 H), 6.96-7.06 (m, 1H), 5.23- 5.35 (m, 1H), 3.51-3.65 (m, 1H), 3.07-3.45 (m, 3H) 2.67-2.70 (s, 3 H), 2.58-2.62 (s, 3 H), 1.41-1.91 (m, 6 H). |

### Example 28

### Determination of Antagonist Affinity at Human Orexin-1 and 2 Receptors Using FLIPR

### Cell Culture

Adherent Chinese Hamster Ovary (CHO) cells, stably expressing the recombinant human Orexin-1 or human Orexin-2 receptors or Rat Basophilic Leukaemia Cells (RBL) stably expressing recombinant rat Orexin-1 or rat Orexin-2 receptors were maintained in culture in Alpha Minimum Essential Medium (Gibco/Invitrogen, cat. no.; 22571-020), supplemented with 10% decomplemented foetal bovine serum (Life Technologies, cat. no. 10106-078) and 400 μg/mL Geneticin G418 (Calbiochem, cat. no. 345810). Cells were grown as monolayers under 95%:5% air:CO₂ at 37° C.

The sequences of the human orexin 1, human orexin 2, rat orexin 1 and rat orexin 2 receptors used in this example were as published in Sakurai, T. et al (1998) Cell, 92 pp 573 to 585. The compounds of some examples (for example the compounds of Examples 1 to 20) were tested against the orexin 1 receptor sequence as published by Sakurai et al supra with the exception that the amino acid residue at position 280 was alanine and not glycine as reported in Sakurai et al.

### Measurement of [Ca²⁺]ᵢ Using the FLIPR™

Cells were seeded into black clear-bottom 384-well plates (density of 20,000 cells per well) in culture medium as described above and maintained overnight (95%:5% air:CO₂ at 37° C.). On the day of the experiment, culture medium were discarded and the cells washed three times with standard buffer (NaCl, 145 mM; KCl, 5 mM; HEPES, 20 mM; Glucose, 5.5 mM; MgCl₂, 1 mM; CaCl₂, 2 mM) added with Probenecid 2.5 mM. The plates were then incubated at 37° C. for 60 minutes in the dark with 2 μM FLUO-4AM dye to allow cell uptake of the FLUO-4AM, which is subsequently converted by intracellular esterases to FLUO-4, which is unable to leave the cells. After incubation, cells were washed three times with standard buffer to remove extracellular dye and 30 μL of buffer were left in each well after washing.

Compounds of the invention were tested in a final assay concentration range from 1.66×10⁻⁵M to 1.58×10⁻¹¹M. Compounds of the invention were dissolved in dimethylsulfoxide (DMSO) at a stock concentration of 10 mM. These stock solutions were serially diluted with DMSO and 1 μL at of each dilution was transferred to a 384 well compound plate. Immediately before introducing compound to the cells, buffer solution (50 μl/well) was added to this plate. To allow agonist stimulation of the cells, a stock plate containing a solution of human orexin A (hOrexin A) was diluted with buffer to final concentration just before use. This final concentration of hOrexin A was equivalent to the calculated EC80 for hOrexinA agonist potency in this test system. This value was obtained by testing hOrexinA in concentration response curve (at least 16 replicates) the same day of the experiment.

The loaded cells were then incubated for 10 min at 37° C. with test compound. The plates were then placed into a FLIPR™ (Molecular Devices, UK) to monitor cell fluorescence (λₑₓ=488 nm, λ_{EM}=540 nm) (Sullivan E, Tucker E M, Dale I L. Measurement of [Ca²⁺]ᵢ using the fluometric imaging plate reader (FLIPR). In: Lambert D G (ed.), Calcium Signaling Protocols. New Jersey: Humana Press, 1999, 125-136). A baseline fluorescence reading was taken over a 5 to 10 second period, and then 10 μL of EC80 hOrexinA solution was added. The fluorescence was then read over a 4-5 minute period.

### Data Analysis

Functional responses using FLIPR were measured as peak fluorescence intensity minus basal fluorescence and expressed as a percentage of a non-inhibited Orexin-A-induced response on the same plate. Iterative curve-fitting and parameter estimations were carried out using a four parameter logistic model and Microsoft Excel (Bowen W P, Jerman J C. Nonlinear regression using spreadsheets. Trends Pharmacol. Sci. 1995; 16: 413-417). Antagonist affinity values (IC₅₀) were converted to functional pKᵢ values using a modified Cheng-Prusoff correction (Cheng Y C, Prusoff W H. Relationship between the inhibition constant (Kᵢ) and the concentration of inhibitor which causes 50 percent inhibition (IC₅₀) of an enzymatic reaction. Biochem. Pharmacol. 1973, 22: 3099-3108).

$$fpKi = - log \frac{( I {C}_{50} )}{{( 2 + {( \frac{[ agonist ]}{( E {C}_{50} )} )}^{n} )}^{1 / n} - 1}$$

Where [agonist] is the agonist concentration, EC₅₀ is the concentration of agonist giving 50% activity derived from the agonist dose response curve and n=slope of the dose response curve. When n=1 the equation collapses to the more familiar Cheng-Prusoff equation.

Compounds of examples 1 to 27 were tested according to the method of example 28. All compounds gave fpKi values from 5.8 to 9.1 at one or both of the human cloned orexin-1 receptor (either as published in Sakurai et al supra or having the amino acid residue alanine at position 280 and not glycine) or the human cloned orexin-2 receptor.

1. A compound of formula (I) 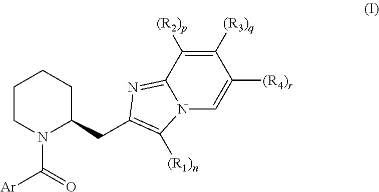
where:
Ar is pyridinyl substituted with one, two or three groups independently selected from the group consisting of C₁₋₄alkyl, halo, C₁₋₄alkoxy, haloC₁₋₄alkyl, haloC₁₋₄alkoxy, cyano, phenyl or a 5 or 6 membered heterocyclyl group containing 1, 2 or 3 atoms selected from N, O or S, which phenyl or heterocyclyl group is optionally substituted with C₁₋₄alkyl, halo, C₁₋₄alkoxy, haloC₁₋₄alkyl, haloC₁₋₄alkoxy or cyano;
R₁ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR⁵R⁶ wherein R⁵ is H or (C₁₋₄)alkyl and R⁶ is H or (C₁₋₄)alkyl;
R₂ is (C₁₋₄)alkyl, (C₁₋₄)alkenyl, HO(C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR⁷R⁸ wherein R⁷ is H or (C₁₋₄)-alkyl and R⁸ is H or (C₁₋₄)-alkyl;
R₃ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR⁹R¹⁰ wherein R⁹ is H or (C₁₋₄)-alkyl and R¹⁰ is H or (C₁₋₄)-alkyl;
R₄ is (C₁₋₄)alkyl, halo, halo(C₁₋₄)alkyl, (C₁₋₄)alkoxy, halo(C₁₋₄)alkoxy, (C₁₋₄)alkyl-O—(C₁₋₄)alkyl, CN, NR¹¹R¹² wherein R¹¹ is H or (C₁₋₄)-alkyl and R¹² is H or (C₁₋₄)-alkyl;
n is 0 or 1;
p is 0 or 1;
q is 0 or 1;
r is 0 or 1;
or a pharmaceutically acceptable salt thereof.

2. A compound according to claim 1 wherein the pyridyl group is linked to the carbonyl group by means of a bond formed between the carbon at the 2 position of the pyridyl and the carbon of said carbonyl group, or a pharmaceutically acceptable salt thereof.

3. A compound according to claim 1 where Ar is substituted with one (C₁₋₄)alkyl group and one (C₁₋₄)alkoxy group, or a pharmaceutically acceptable salt thereof.

4. A compound according to claim 3 where Ar is substituted with one methyl group and one (C₁₋₄)alkoxy group, or a pharmaceutically acceptable salt thereof.

5. A compound according to claim 1 where Ar is substituted with one (C₁₋₄)alkyl group and one propoxy, ethoxy, methoxy, methylethoxy, methylpropoxy or cyclopropylmethoxy group, or a pharmaceutically acceptable salt thereof.

6. A compound according to claim 5 where Ar is substituted with one methyl group and one propoxy, ethoxy, methoxy, methylethoxy, methylpropoxy or cyclopropylmethoxy group, or a pharmaceutically acceptable salt thereof.

7. A compound according to claim 1 wherein Ar is substituted with one (C₁₋₄)alkyl group and one phenyl group, or a pharmaceutically acceptable salt thereof.

8. A compound according to claim 7 where Ar is substituted with one methyl group and one phenyl group, or a pharmaceutically acceptable salt thereof.

9. A compound according to claim 1 where n is 0, p is 1, q is 1, r is 0, R₂ is alkyl, R₃ is alkyl and Ar is substituted with one (C₁₋₄)alkyl group and one (C₁₋₄)alkoxy group, or a pharmaceutically acceptable salt thereof.

10. A compound according to claim 9 where R₂ is methyl, R₃ is methyl and Ar is substituted with one methyl group and one propoxy group, or a pharmaceutically acceptable salt thereof.

11. A compound according to claim 1 where n is 0, p is 1, q is 0, r is 1, R₂ is (C₁₋₄)alkyl, R₄ is halo and Ar is substituted with one (C₁₋₄)alkyl group and one phenyl group, or a pharmaceutically acceptable salt thereof.

12. A compound according to claim 11 where R₂ is methyl, R₄ is fluoro and Ar is substituted with one methyl group and one phenyl group, or a pharmaceutically acceptable salt thereof.

13. A compound according to claim 1 where n is 1, p is 1, q is 0, r is 0, R₁ is halo, R₂ is (C₁₋₄)alkyl and Ar is substituted with one (C₁₋₄)alkyl group and one cyclopropoxymethyl group, or a pharmaceutically acceptable salt thereof.

14. A compound according to claim 13 where R₁ is chloro, R₂ is methyl and Ar is substituted with one methyl group and one cyclopropoxymethyl group, or a pharmaceutically acceptable salt thereof.

15. A compound selected from:
2-[((2S)-1-{[3-(ethyloxy)-6-methyl-2-pyridinyl]carbonyl}-2-piperidinyl)methyl]-6-fluoro-8-methylimidazo[1,2-a]pyridine;
6-fluoro-8-methyl-2-{[(2S)-1-({6-methyl-3-[(2-methylpropyl)oxy]-2-pyridinyl}carbonyl)-2-piperidinyl]methyl}imidazo[1,2-c]pyridine;
6,8-dimethyl-2-{[(2S)-1-({6-methyl-3-[(2-methylpropyl)oxy]-2-pyridinyl}carbonyl)-2-piperidinyl]methyl}imidazo[1,2-c]pyridine;
8-methyl-2-[((2S)-1-{[6-methyl-3-(propyloxy)-2-pyridinyl]carbonyl}-2-piperidinyl)methyl]imidazo[1,2-a]pyridine;
2-{[(2S)-1-({3-[(cyclopropylmethyl)oxy]-6-methyl-2-pyridinyl}carbonyl)-2-piperidinyl]methyl}-8-methylimidazo[1,2-a]pyridine;
8-methyl-2-{[(2S)-1-({6-methyl-3-[(1-methylethyl)oxy]-2-pyridinyl}carbonyl)-2-piperidinyl]methyl}imidazo[1,2-a]pyridine;
2-[((2S)-1-{[4-chloro-3-(ethyloxy)-6-methyl-2-pyridinyl]carbonyl}-2-piperidinyl)methyl]-8-methylimidazo[1,2-a]pyridine;
7,8-dimethyl-2-{[(2S)-1-({6-methyl-3-[(2-methylpropyl)oxy]-2-pyridinyl}carbonyl)-2-piperidinyl]methyl}imidazo[1,2-c]pyridine;
2-{[(2S)-1-({3-[(cyclopropylmethyl)oxy]-6-methyl-2-pyridinyl}carbonyl)-2-piperidinyl]methyl}-7,8-dimethylimidazo[1,2-c]pyridine;
2-[((2S)-1-{[3-(ethyloxy)-6-methyl-2-pyridinyl]carbonyl}-2-piperidinyl)methyl]-7,8-dimethylimidazo[1,2-c]pyridine;
7,8-dimethyl-2-[((2S)-1-{[6-methyl-3-(propyloxy)-2-pyridinyl]carbonyl}-2-piperidinyl)methyl]imidazo[1,2-a]pyridine;
8-fluoro-2-[((2S)-1-{[6-methyl-3-(propyloxy)-2-pyridinyl]carbonyl}-2-piperidinyl)methyl]imidazo[1,2-c]pyridine;
8-fluoro-2-{[(2S)-1-({6-methyl-3-[(2-methylpropyl)oxy]-2-pyridinyl}carbonyl)-2-piperidinyl]methyl}imidazo[1,2-c]pyridine;
2-{[(2S)-1-({3-[(cyclopropylmethyl)oxy]-6-methyl-2-pyridinyl}carbonyl)-2-piperidinyl]methyl}-8-fluoroimidazo[1,2-c]pyridine;
6,7-dimethyl-2-{[(2S)-1-({6-methyl-3-[(2-methylpropyl)oxy]-2-pyridinyl}carbonyl)-2-piperidinyl]methyl}imidazo[1,2-a]pyridine;
3-chloro-2-{[(2S)-1-({3-[(cyclopropylmethyl)oxy]-6-methyl-2-pyridinyl}carbonyl)-2-piperidinyl]methyl}-8-methylimidazo[1,2-a]pyridine;
3-chloro-2-[((2S)-1-{[3-(ethyloxy)-6-methyl-2-pyridinyl]carbonyl}-2-piperidinyl)methyl]-8-methylimidazo[1,2-a]pyridine;
2-{[(2S)-1-({3-[(cyclopropylmethyl)oxy]-6-methyl-2-pyridinyl}carbonyl)-2-piperidinyl]methyl}-3,8-dimethylimidazo[1,2-a]pyridine;
2-[((2S)-1-{[6-ethyl-3-(ethyloxy)-2-pyridinyl]carbonyl}-2-piperidinyl)methyl]-7,8-dimethylimidazo[1,2-a]pyridine;
6-fluoro-8-methyl-2-({(2S)-1-[(6-methyl-3-phenyl-2-pyridinyl)carbonyl]-2-piperidinyl}methyl)imidazo[1,2-c]pyridine;
7,8-dimethyl-2-[((2S)-1-{[6-methyl-3-(3-methyl-1,2,4-oxadiazol-5-yl)-2-pyridinyl]carbonyl}-2-piperidinyl)methyl]imidazo[1,2-a]pyridine;
7,8-dimethyl-2-[((2S)-1-{[6-methyl-3-(5-methyl-1,3-oxazol-2-yl)-2-pyridinyl]carbonyl}-2-piperidinyl)methyl]imidazo[1,2-a]pyridine;
2-[((2S)-1-{[3-(5-ethyl-1,3-oxazol-2-yl)-6-methyl-2-pyridinyl]carbonyl}-2-piperidinyl)methyl]-7,8-dimethylimidazo[1,2-a]pyridine;
7,8-dimethyl-2-[((2S)-1-{[6-methyl-3-(2-pyrimidinyl)-2-pyridinyl]carbonyl}-2-piperidinyl)methyl]imidazo[1,2-a]pyridine;
7,8-dimethyl-2-[((2S)-1-{[6-methyl-3-(3-methyl-5-isoxazolyl)-2-pyridinyl]carbonyl}-2-piperidinyl)methyl]imidazo[1,2-a]pyridine;
7,8-dimethyl-2-[((2S)-1-{[6-methyl-3-(4-methyl-1,3-thiazol-2-yl)-2-pyridinyl]carbonyl}-2-piperidinyl)methyl]imidazo[1,2-a]pyridine; and
6-fluoro-8-methyl-2-[((2S)-1-{[6-methyl-3-(2-pyrimidinyl)-2-pyridinyl]carbonyl}-2-piperidinyl)methyl]imidazo[1,2-c]pyridine;
or a pharmaceutically acceptable salt thereof.

16-24. (canceled)

25. A method for the treatment of a disease or disorder where an antagonist of a human orexin receptor is required, in a subject in need thereof, comprising administering to said subject an effective amount of a compound as defined in claim 1, or a pharmaceutically acceptable salt thereof, wherein the disease or disorder is a sleep disorder, a depression or mood disorder, an anxiety disorder, a substance-related disorder or a feeding disorder.

26. (canceled)

27. A method according to claim 26 where the disease or disorder is a sleep disorder.

28. A method according to claim 27 where the sleep disorder is selected from the group consisting of Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; Sleep Apnea and Jet-Lag Syndrome.

29. A pharmaceutical composition comprising a) the compound as defined in claim 1, or a pharmaceutically acceptable salt thereof, and b) one or more pharmaceutically acceptable carriers.
